(19) United States
(12) Patent Application Publication
(10) Pub. No.: US 2025/0127707 A1
(43) Pub. Date: Apr. 24, 2025

(54) FRAGRANCE FORMULATION

(71) Applicant: Coty Inc., New York, NY (US)

(72) Inventors: Marta Alexandra de Sousa Oliveira Maia, Versoix (CH); Sarah Kyakyo Kanyunuzi Nyakana, Versoix (CH); Mariano Agustin Rodriguez-Fraticelli, Versoix (CH); Jose Maria Velazquez Mendoza, Nyon (CH)

(21) Appl. No.: 18/695,700

(22) PCT Filed: Sep. 29, 2022
(86) PCT No.: PCT/US2022/045203

§ 371 Date: Mar. 26, 2024

Related U.S. Application Data
(60) Provisional application No. 63/366,727, filed on Jun. 21, 2022.
(60) Provisional application No. 63/250,891, filed on Sep. 30, 2021.

(51) Int. Cl. A61K 8/87; A61K 8/34; A61Q 13/00

(52) U.S. Cl. CPC A61K 8/87; A61K 8/34; A61Q 13/00

(57) ABSTRACT

The present application is directed to various fragrance compositions comprising polyurethane, especially polyurethane-64, and a fragrance. It is also directed to a primer based polyurethane-64, ethanol, water and a modulator.

### CLAIM FOR PRIORITY

This patent application claims the benefit of priority to U.S. Application Ser. No. 63/366,727, filed Jun. 21, 2022, and U.S. Application Ser. No. 63/250,891, filed Sep. 30, 2021, which are incorporated by reference herein in their entirety.

### FIELD

Inventive subject matter herein relates to the field of perfumery. In particular, inventive embodiments disclosed herein relate to fragrance formulations having enhanced longevity and stability.

### BACKGROUND

A fragrance is like a symphony of scents. Once a fragrance is applied to skin, a sensory experience is initiated that changes over time. It is known to encapsulate perfume oils in liposomes or cyclodextrins to achieve a controlled release of these oils. Several systems have also been used to improve bonding of perfume oils to the skin or to extend the retention time of perfume on skin. These systems have included chitin derivatives, quaternary chitosans, silicate materials and thermoplastic polyamides.

### IN THE DRAWINGS

FIG. 1 is a graphical view of base notes after three hours in a fragrance that includes polyurethane-64 in several concentrations, compared to a fragrance free from polyurethane-64.

FIG. 2 is a graphical view of top notes after three hours in a fragrance that includes polyurethane-64 in several concentrations, compared to a fragrance free from polyurethane 64.

FIG. 3 is a graphical view of perfume residue on a slide, comparing residue of perfume formulations that include polyurethane-64 to perfume formulations that are free from polyurethane-64.

FIG. 4 is a graphical view of perfume residue after four hours on a slide, comparing residue of perfume formulations that include polyurethane-64 to perfume formulations that are free from polyurethane-64.

### SUMMARY

One embodiment is a fragrance formulation that includes ethanol, water, one or more fragrances and a high molecular weight ingredient with at least one carbamate linkage, resulting from the reaction of an isocyanate and a polyol, the reaction comprising:

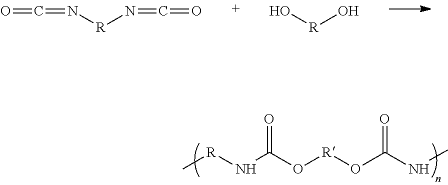

Another embodiment includes a fragrance formulation that includes ethanol and polyurethane-64, and one or more fragrances.
1. Another embodiment includes a fragrance formulation that includes an alcohol and a water insoluble urethane copolymer formed by a multi-step reaction wherein isophorone diisocyanate (IDPI) is reacted with a mixture of polytetrahydrofurans (PTHFs), also known as polybutylene glycols or polytetramethylene glycol such that one of the PTHFs contains an average of 14 mols and the other an average of 28 mols of butylene glycol further wherein a resulting polyurethane is reacted with 4,4′-methylenebis(cyclohexylamine) and residual isocyanate groups are reacted with ethanol.

Another embodiment includes a fragrance formulation consisting of:

| Ingredient | Wt. % |
|---|---|
| Alcohol | qs 100 |
| Fragrance | 5.0-20.0 |
| Water | 0.5-16.0 |
| UV filters | 0.05-3.0 |
| Polyurethane-64 | 0.04-3.0 |

wherein the polyurethane-64 is a high molecular weight ingredient with at least one carbamate linkage, resulting from the reaction of an isocyanate and a polyol

Another embodiment includes a fragrance formulation, that includes water; UV filters; ethanol and polyurethane-64; a modulator component in a range of from about 0.1 wt % to about 30 wt % of the fragrance composition, the modulator being substantially non-odorous; and a fragrance component present in an amount in a range of from about 0.04 wt % to about 40 wt % of the fragrance composition, the fragrance component comprising at least one of: at least one low volatile fragrance material having a vapor pressure less than 0.001 Torr (0.000133 kPa) at 25° C.; at least one moderate volatile fragrance material having a vapor pressure in the range of 0.1 Torr to 0.001 Torr (0.0133 kPa to 0.000133 kPa) at 25° C.; and at least one high volatile fragrance material having a vapor pressure greater than 0.1 Torr (0.0133 kPa) at 25° C., wherein the polyurethane 64 is a high molecular weight ingredient with at least one carbamate linkage, resulting from the reaction of an isocyanate and a polyol, the reaction comprising:

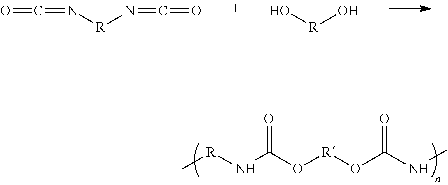

### DETAILED DESCRIPTION

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only; they do not exclude other defined values or other values within defined ranges for the radicals and substituents.

### Definitions

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “apply” or “application,” as used in reference to a composition, means to apply or spread the compositions of the present invention onto keratinous tissue such as the epidermis.

The term “keratinous tissue” refers to keratin-containing layers disposed as the outermost protective covering of mammals (e.g., humans, dogs, cats, etc.) which includes, but is not limited to, skin, lips, hair, toenails, fingernails, cuticles, hooves, etc.

The terms “comprises,” “comprising,” and the like can have the meaning ascribed to them in U.S. Patent Law and can mean “includes,” “including” and the like. As used herein. “including” or “includes” or the like means including, without limitation.

As used herein, the terms “include”, “includes” and “including” are meant to be non-limiting.

For inventive embodiments herein, any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

For inventive embodiments herein, a “monomer” is a low-molecular weight compound comprising functional moieties, wherein said monomer functions as a building block for polymers and has a defined molecular weight.

In the present patent application, the term “polymer” refers to a compound, formed during a chemical reaction by linking several monomers (i.e. more than two monomers) of the same or different kind together via covalent bonding, wherein the resulting polymer can differ in its degree of polymerization, molecular weight distribution and chain length respectively. Hence, a polymer according to the present invention is a compound, comprising in its molecular structure at least one repeating unit, which was integrated in the polymer structure during polymer synthesis by repeatedly linking monomers together via covalent bonds to form said polymer structure. The number average molecular weight is preferably at least 250 g/mol, more preferably at least 1,000 g/mol.

The term “polymer” includes homopolymers, copolymers, block-copolymers and oligomers.

In the present application, a “prepolymer” is a polymer with reactive groups. In analogy to the definition of the term “polymer”, the molecular structure of a prepolymer is formed by repeatedly linking more than two monomers of the same or different kind together. The prepolymer can participate in a subsequent formation of a polymer, which has a higher molecular weight than said prepolymer. The term “prepolymer” encompasses polymers, which are able to chemically react via at least one of its reactive groups, forming a repeating unit of a (preferably crosslinked) polymer. Therefore, the term “prepolymer” encompasses as well self-crosslinking polymers with at least two different kinds of reactive groups, wherein said groups are able to chemically react among themselves, so that the prepolymer molecules are able to crosslink.

As used herein, the term “composition” includes a fine fragrance composition intended for application to a body surface, such as for example, skin or hair, e.g., to impart a pleasant odor thereto, or cover a malodor thereof. They are generally in the form of perfume concentrates, perfumes, eaux de parfum, eaux de toilette, aftershaves, or colognes. The fine fragrance compositions may be an ethanol-based composition. The term “composition” may also include a cosmetic composition, which comprises a fragrance material for the purposes of delivering a pleasant smell to drive consumer acceptance of the cosmetic composition. The term “composition” may also include body splashes or body sprays. The term “composition” may also include cleaning compositions, such as fabric care composition or home care compositions, including air care compositions (e.g., air fresheners), for use on clothing or other substrates such as hard surfaces (e.g., dishes, floors, countertops). Additional non-limiting examples of “composition” may also include facial or body powder, deodorant, foundation, body/facial oil, mousse, creams (e.g., cold creams), waxes, sunscreens and blocks, bath and shower gels, lip balms, self-tanning compositions, masks and patches.

As used herein, the term “consumer” means both the user of the composition and the observer nearby or around the user.

As used herein, the term “fragrance material” and “fragrance materials” relates to a perfume raw material (“PRM”), or a mixture of perfume raw materials (“PRMs”), that are used to impart an overall pleasant odor or fragrance profile to a composition. “Fragrance materials” can encompass any suitable perfume raw materials for fragrance uses, including materials such as, for example, alcohols, aldehydes, ketones, esters, ethers, acetates, nitriles, terpene hydrocarbons, nitrogenous or sulfurous heterocyclic compounds and essential oils. However, naturally occurring plant and animal oils and exudates comprising complex mixtures of various chemical components are also known for use as “fragrance materials”. The individual perfume raw materials which comprise a known natural oil can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994). Additionally, some perfume raw materials are supplied by the fragrance houses (Firmenich, International Flavors & Fragrances, Givaudan, Symrise) as mixtures in the form of proprietary specialty accords. Non-limiting examples of the fragrance materials useful herein include pro-fragrances such as acetal pro-fragrances, ketal pro-fragrances, ester pro-fragrances, hydrolyzable inorganic-organic pro-fragrances, and mixtures thereof. The fragrance materials may be released from the pro-fragrances in a number of ways. For example, the fragrance may be released as a result of simple hydrolysis, or by a shift in an equilibrium reaction, or by a pH-change, or by enzymatic release.

As used herein, the term “fragrance profile” means the description of how the fragrance is perceived by the human nose at any moment in time. The fragrance profile may change over time. It is a result of the combination of the low, moderate and high volatile fragrance materials, if present, of a fragrance. A fragrance profile is composed of two characteristics: ‘intensity’ and ‘character’. The ‘intensity’ relates to the perceived strength whilst ‘character’ refers to the odor impression or quality of the perfume, e.g., fruity, floral, woody, etc.

As used herein, the terms “modulator”, and “fragrance modulator” are used interchangeably to designate an agent having the capacity to affect the fragrance profile, such as for example, by impacting the fragrance materials' evaporation rate. The modulator may mediate its effect by lowering the vapor pressure of the fragrance materials and increasing their adherence to the substrate (skin and/or hair) thus ensuring a longer-lasting impression of the fragrance. By incorporating the modulator, it is desired that the fragrance profile, preferably the fragrance components composition attributable to the high and moderate volatile fragrance materials, alone or individually, of the composition can be perceived by a panel of experts or professional evaluators or individual experts or professional evaluators, over a longer period of time, or the perceived harshness of overdosing of the fragrance material is mitigated or absent, as compared to the same perception in the absence of the modulator. As used herein “overdose” can include overdosing a moderate volatile component or high volatile component in aggregate (e.g., greater than 30 wt % of the fragrance component). The term “overdose” can further include overdosing an individual component of the moderate volatile component or the high volatile component (e.g., if the high volatile component includes three oils at least one of the oils may account for a greater wt % of the high volatile component than would be present in a traditional fragrance or a fragrance that is free of the modulators described herein). Suitable examples of the modulator are provided herein below

As used herein, the term “substantially non-odorous” means an agent that does not impart an odor of its own when added into a composition of the present invention. For example, a “substantially non-odorous fragrance modulator” does not impart a new odor that alters the character of the fragrance profile of the composition to which it is added. The term “substantially non-odorous” also encompasses an agent that may impart a minimal or slight odor of its own when added into a composition of the present invention. However, the odor imparted by the “substantially non-odorous fragrance modulator” is generally undetectable or tends to not substantively alter the character of the fragrance profile of the composition to which it is added initially or preferably over time. Furthermore, the term “substantially non-odorous” also includes materials that are perceivable only by a minority of people or those materials deemed “anosmic” to the majority of people. Furthermore, the term “substantially non-odorous” also includes materials that may, from particular suppliers, contain an odor due to impurities, such as when the materials contain the impurities at not more than about 5 wt %, preferably not more than 1 wt %, often even not more than 1 part per million (ppm). These impurities maybe removed by purification techniques known in the art as required to make them suitable for use in fragrance compositions of the present invention.

As used herein, the term “vapor pressure” means the partial pressure in air at a defined temperature (e.g., 25° C.) and standard atmospheric pressure (760 mmHg) for a given chemical species. It defines a chemical species' desire to be in the gas phase rather than the liquid or solid state. The higher the vapor pressure the greater the proportion of the material that will, at equilibrium, be found in a closed headspace. It is also related to the rate of evaporation of a fragrance material which is defined in an open environment where material is leaving the system. The vapor pressure is determined according to the reference program Advanced Chemistry Development (ACD/Labs) Software Version 14.02, or preferably the latest version update).

In all embodiments of the present invention, all percentages are by weight of the total composition, as evident by the context, unless specifically stated otherwise. All ratios are weight ratios, unless specifically stated otherwise, and all measurements are made at 25° C., unless otherwise designated.

### Compositions:

Inventive embodiments disclosed herein include a fragrance formulation that includes an alcohol such as ethanol, polyurethane-64, and water, as well as one or more fragrances having a range of notes. The polyurethane-64 traps these fragrance notes and improves the longevity of the fragrance compared to formulations that do not include polyurethane-64. The polyurethane-64 also aids in avoiding the degradation of some fragrance raw materials that decrease coloration of the fragrance after storage at higher temperatures. The polyurethane-64 acts as a film former with fixative properties.

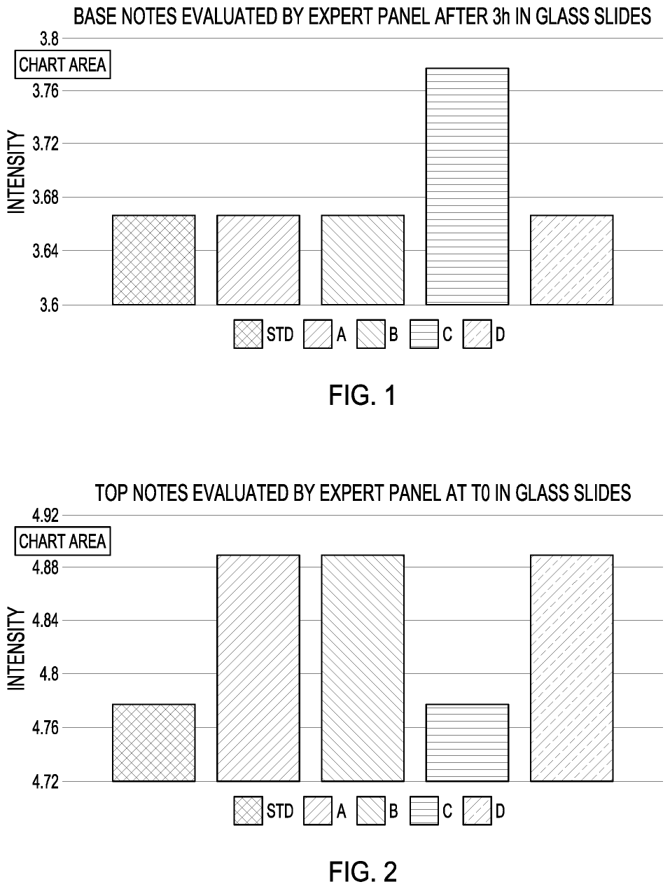

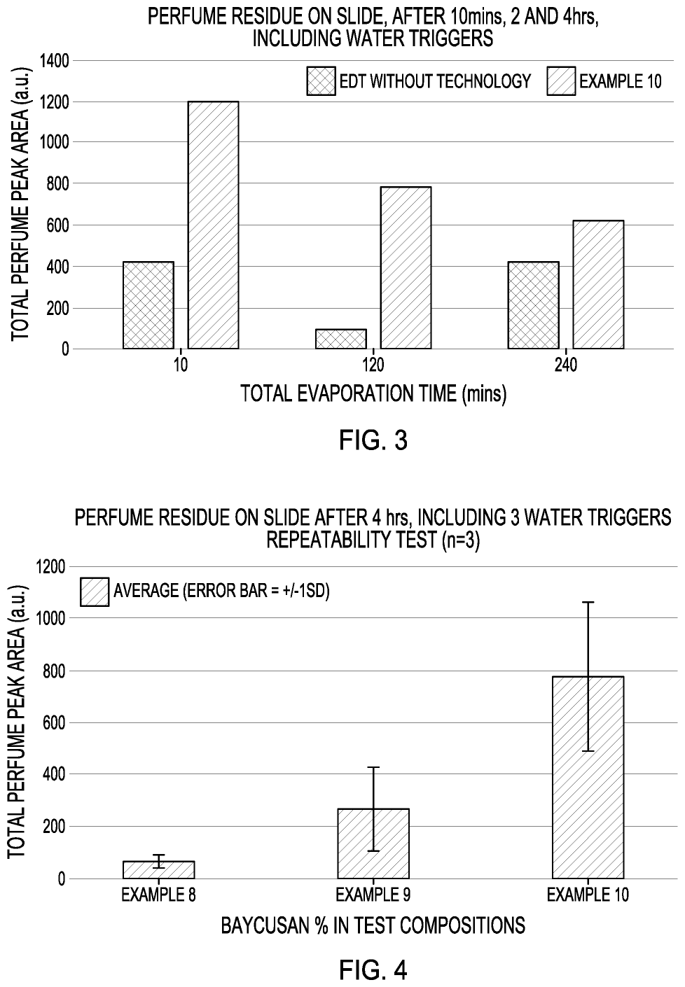

The presence of polyurethane-64 was shown to extend the lifetime of a citrus/cologne character in a fragrance beyond a two hour period after application. It was found that a fragrance having the combination of glucam and polyurethane-64 displayed an extension of the citrus/cologne character to at least six hours. FIGS. 1 and 2 show that the concentration of fragrance oil is usable with polyurethane-64 without compromising the performance of the fragrance. FIGS. 3 and 4 illustrate waterproof properties of fragrance formulations disclosed herein. Fragrance formulations that include polyurethane-64 have a larger fragrance residue compared to formulations that do not include polyurethane-64.

Inventive formulation embodiments display uniform flexible films when applied to wet or dry skin. Thus, the polyurethane-64 stabilizes the appearance of the fragrance and prevents discoloration.

The term, “polyurethane-64” as used herein refers to a high molecular weight ingredient with at least one carbamate linkage, resulting from the reaction of an isocyanate and a polyol.

In one embodiment, Polyurethane-64 is a urethane copolymer formed by a multi-step reaction. First, isophorone diisocyanate (IDPI) is reacted with a mixture of polytetrahydrofurans (PTHFs), also known as polybutylene glycols or polytetramethylene glycols. One of the PTHFs contains an average of 14 mols and the other an average of 28 mols of butylene glycol. The resulting polyurethane is reacted with 4,4′-methylenebis(cyclohexylamine) and finally the residual isocyanate groups are reacted with ethanol. Reactant monomer structures are as follows:
Polyurethane-64 Polyurethane-64 is a urethane copolymer formed by a multi-step reaction. First, isophorone diisocyanate (IDPI) is reacted with _Film former a mixture of polytetrahydrofurans (PTHFs), also know as polybutylene glycols or polytetramethylene glycols. One of the PTHFs contains an average of 14 mols and the other an average of 28 mols of butylene glycol. The resulting polyurethane is reacted with 4,4′-methylenebis(cyclohesylamine) and finally the residual isocyanate groups are reacted with ethanol. [Monomers/reactants;]

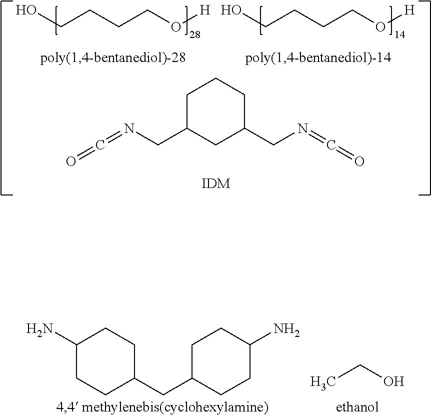

polyurethanes.pdf(cir-safety.org)

Polyurethane-64 is made by Covestro, with headquarters in Leverkusen, DE, and is sold as Baycusan C2000. The Baycusan C2000 formulation includes between 35% to 45% non-volatile content, i.e. polyurethane-64, (40% average), so the percentage of polyurethane-64 added to the fragrance compositions was 0.24% to 0.60% for better performance.

The Baycusan C2000 films have a tensile strength at 100% elongation of about 6 mPaS, an elongation at break of about 550% and a tensile strength at break of about 18 mPa·S. This Baycusan C2000 formulation has a percent solids of 40.0+/−5 percent and is a clear, transparent solution. Baycusan C2000 concentration range is 0.1 to 7.0% by weight in the fragrance formulation embodiments herein. The fragrance formulation embodiments also include an alcohol in addition to the alcohol in the Baycusan C2000, such as ethanol, or an alcohol/water mixture.

Another polyurethane binder and film former is polyurethane-1. Polyurethane-1 is a copolymer of isophthalic acid, adipic acid, hexylene glycol, neopentyl glycol, dimethylolpropanoic acid, and isophorone diisocyanate monomers:

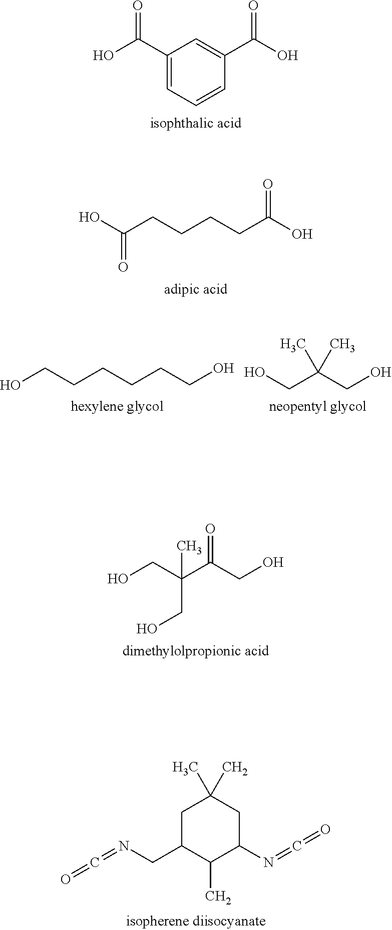

Polyurethane-1 is sold under following names: Luviset P.U.R, manufactured by BASF, Daitosol U9-30 and Daitosol U9-40, manufactured by Daito Kasei Kogyo, Spherepoly Hard and Spherepoly Medium, manufactured by The Innovation Company.

Another polyurethane binder is Polyurethane-35. Polyurethane-35 is a complex polymer that is made by reacting a polyester pre-polymer that includes adipic acid, hexanediol, and neopentyl glycol with dicyclohexymethane diisocyanate. The resulting urethane polymer is further react with sodium N-(2-aminoethyl)-3-aminoethyl)-3-aminoethanesulfonate and ethylenediamine and then dispersed into water. Monomers include:

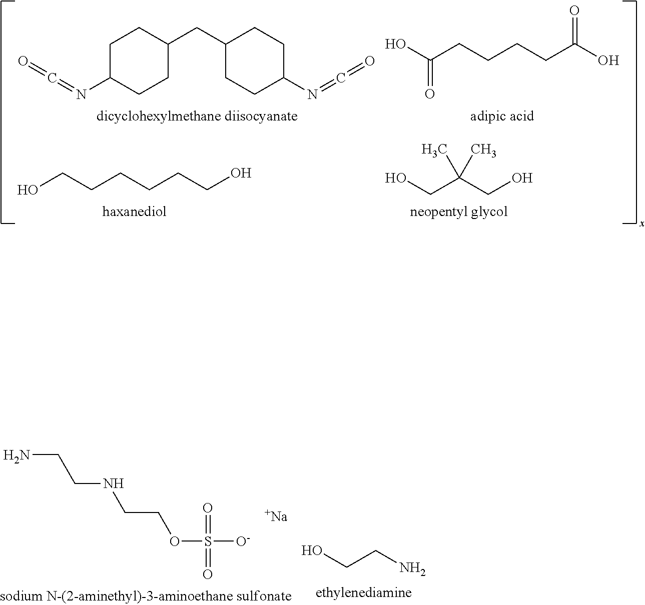

Polyurethane35 is made by Covestro and sold at Baycusan C 1004. The Baycusan C 1004 is a colloidal system of high molecular weight polyurethane polymer dispersed in water. The polymer structure contains both hydrophilic and hydrophobic segments, which impart a unique combination of water resistance and ease of removal from skin. It imparts non-transfer resistant properties to decorative cosmetics. It displays a good balance of properties like great aesthetics, non-greasy and non-sticky feel on the skin, excellent spreadability, good adhesion to skin, fast drying, excellent abrasive resistance and good washability. It is a biocide-free product suitable for various emulsions type and cold processes. It combines safe and easy application with comfortable long-term protection. It is gentle and suitable for matured & sensitive skin. Baycusan® C 1004 can be incorporated into oil-in-water and water-in-oil emulsions as well as in multiple emulsions, gels and gel creams. Used in skin care, sun care and color cosmetics formulations.

The Baycusan C 1004 has the following physical properties:

| Property | Value and Unit | |
|---|---|---|
| Solids | 39-43 | wt. % |
| Viscosity | Less than 500 mPa-s | |
| pH | 6.5-8.5 | |
| Glass Transition | −45.5 | degrees C. |
| Tensile Strength | 4.0 | MPa |

| | |
|---|---|
| Elongation at Break | 1550% |
| Tensile Strength | 40 MPa at break |
| Swelling in Water | 0% |

In some embodiments, the polyurethanes include at least one water-soluble or water-dispersible polymer having a surface tension of less than about 75γ. The polymers exhibit good water-resistance, adhesion and flexibility on dry down.

A pH of the fragrance composition can be in a range of from about 4 to about 8, about 5 to about 7, less than, equal to, or greater than about 4, 5, 6, 7, or about 8. The ethanol concentration in any of the compositions described herein can be less than about 78 wt % ethanol, less than about 70 wt % ethanol, less than about 60 wt % ethanol, less than about 50 wt % ethanol, less than about 40 wt % ethanol, less than about 30 wt % ethanol, less than about 20 wt % ethanol, less than about 10 wt % ethanol, or free of ethanol.

Some fragrance formulation embodiments include water. Fragrance concentrations in the fragrance formulation embodiments disclosed herein range from 0.01 to about 16% w/w.

One example of a fragrance formulation having an improved stability and longevity is as follows:

| Ingredient | Wt. % |
|---|---|
| Alcohol | qs 100 |
| Fragrance | 5.0-20.0 |
| Water | 0.5-16.0 |
| UV filters | 0.5-3.0 |
| Polyurethane-64 | 0.04-3.0 |

Fragrance formulation embodiments include lotions, sprays, gels, oils, pump sprays and aerosols.

Some fragrance embodiments include additional materials such as the following:

Oils: Silicone oils, mineral oils, Hydrogenated Polyisobutene, Polyisoprene, Squalane, Tridecyl Trimellitate, Trimethylpropane Triisostearate, Isodecyl Citrate, Neopentyl Glycol Diheptanoate, PPG-15 Stearyl Ether as well as vegetable oils such as Calendula Oil, Jojoba Oil, Avocado Oil, Macadamia Nut Oil, Castor Oil, Cocoa Butter, Coconut Oil, Maize Oil, Cottonseed Oil, Olive Oil, Palm Kernel Oil, Rapeseed Oil, Safflower Oil, Sesame Seed Oil, Soybean Oil, Sunflower Seed Oil, Wheatgerm Oil, Grapeseed Oil, Kukui Nut Oil, Thistle Oil, and mixtures thereof. Synthetic squalane or squalane made from natural products is suitable too, as well as cosmetic esters or ethers which can be branched or linear, saturated or unsaturated.

Scavengers: Antioxidants, vitamins such as Vitamin C and derivatives thereof, e.g. ascorbyl acetate, ascorbyl phosphate and ascorbyl palmitate; Vitamin A and derivatives thereof, folic acid and derivatives thereof; Vitamin E and derivatives thereof such as tocopheryl acetate; flavones and flavonoids; amino acids such as histidine, glycine, tyrosine, tryptophan and derivatives thereof; carotenoids and carotenes such as α-carotene, β-carotene; uric acid and derivatives thereof; α-hydroxy acids such as citric acid, lactic acid, malic acid.

Moisturizing substances: Glycerine, Butylene Glycol, Propylene Glycol, and mixtures thereof.

Organic sunscreens: Derivatives of 4-aminobenzoic acid such as 4-(dimethylamino)-benzoic acid-(2-ethylhexyl)ester; esters of cinnamic acid such as 4-methoxy cinnamic acid(2-ethylhexyl)Ester; benzophenone derivatives such as 2-Hydroxy-4-methoxy benzophenone; derivatives of 3-benzylidene camphor such as 3-Benzylidene Camphor. Other preferred oil-soluble UV filters are Benzophenone-3, Butyl Methoxybenzoylmethane, Octyl Methoxycinnamate, Octyl Salicylate, 4-Methylbenzylidene Camphor, Homosalate, and Octyl Dimethyl PABA.

Surface-active agents: Anionic, amphoteric, non-ionic or cationic surface-active agents, or mixtures thereof. Cationic polymers or a mixture of anionic and amphoteric surface-active agents are particularly preferred.

Non-limiting examples of anionic foaming surface-active agents include those selected from the group consisting of alkyl sulphates, alkyl ether sulphates, sulphated monoglycerides, sulphonated olefins, alkyl aryl sulphonates, primary or secondary alkane sulphonates, alkyl sulphosuccinates, acyl taurates, acyl isothionates, alkyl glyceryl ether sulphonates, sulphonate methyl esters, sulphonated fatty acids, alkyl phosphates, acyl glutamates, acyl sarcosinates, alkyl sulpho-acetates, acylated peptides, alkyl ether carboxylates, acyl lactylates of anionic surface-active agents containing fluorine, and mixtures thereof. Mixtures of anionic surface-active agents can be effectively used in the present invention.

Examples of amphoteric surface-active agents which can be used in the present invention include at least those having an acid group. The aforesaid group can be a carboxyl group or a sulphonic acid group. Quaternary nitrogen and therefore quaternary amino acids are included. They should, in general, contain an alkyl group or alkenyl group having 7 to 18 carbon atoms. Suitable amphoteric detergents include simple betaines and amidobetaines which are a mixture of C12- and C14-alkyl groups derived from the coconut so that at least half, preferably three quarters, of the R1-hydrocarbon chain has 10 to 14 carbon atoms. It is preferred that the other two R2- and R3-hydrocarbon chains be methyl. Further, the amphoteric detergent can be a sulphobetaine. Amphoacetates and diamphoacetates can also occur as possible zwitterionic and/or amphoteric compounds, which can be used. An amphoteric surface-active agent should, in general, be contained in an amount ranging approximately between 0.1 and 20%, preferably 5 and 18% by weight, relative to the composition.

Suitable non-ionic surface-active agents include, but are not limited to, Coconut Acyl Mono-ethanolamide or Coconut Acyl Diethanolamide, Alkyl Polysaccharide, Lactobionamide, Ethylene Glycol Ester, Glycerine Monoether, Polyhydroxyamide (Glucamide), primary and secondary alcohol ethoxylates, particularly C₈₋₂₀ aliphatic alcohols ethoxylated with an average of 1 to 20 moles ethylene oxide per mole of alcohol. Mixtures of the aforesaid surface-active agents can also be used.

Some embodiments also include one or more colors. Pigments, pigment mixtures or powders with a pigment-like effect, also including those with a pearl-gloss effect may be added to the composition of the invention. The may include, for example, iron oxides, aluminum silicates such as ochre, titanium (di)oxide, mica, kaolin, manganese containing clays such as umber and red bole, calcium carbonate, French chalk, mica-titanium oxide, mica-titanium oxide-iron oxide, bismuth oxychloride, nylon beads, ceramic beads, expanded and non-expanded synthetic polymer powders, powdery natural organic compounds such as milled solid algae, milled plant parts, encapsulated and non-encapsulated cereal starches and mica-titanium oxide-organic dye. Also included are chalk, black pigments, pearlescent pigments, and fluorescent or phosphorescent pigments.

Some formulation embodiments include antiperspirants and deodorants, such as Triclosan, Trimethyl Citrate, Farnesol, Aluminum Chlorhydrate, Aluminum Zirconium Tetrachlorhydex GLY etc.

Fragrances having a range of notes include a wide variety of fragrance materials. Fragrance imparting materials employed in fragrance formulation embodiments disclosed herein include individual fragrance compounds, such as synthetic products of esters, ethers, aldehydes, ketones, alcohols and hydrocarbons. Fragrance compounds of the ester type include benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allylcyclohexyl propionate, styrallyl propionate and benzyl salicylate. Fragrance imparting materials also include perfume oils that also contain mixtures of natural odoriferous substances obtained from vegetable or animal sources. Essential oils of lower volatility, which are used mostly as flavor components, are also suitable as perfume oils.

The fragrance materials can be grouped in terms of their volatility. Generally, the materials can be grouped as low volatile fragrance materials, moderate volatile fragrance materials, and high volatile fragrance materials. Each group of materials can be associated with various perceptions by a panel of experts or professional evaluators or individual experts or professional evaluators. While not so limited, a high volatile fragrance may be associated with a citrus character; a moderate volatile fragrance may be associated with a spicy character; and a low volatile fragrance may be associated with a woody character. Each group of fragrance materials can include synthetic materials or natural materials. The volatility of the fragrance materials can be with reference to an individual fragrance material. Alternatively, in cases where a combination of materials produce a fragrance, for example a natural oil, the volatility may be with reference to that aggregation.

In some examples, this disclosure shows that longer lasting fragrance profiles or at least initial fragrance profiles, may be enhanced through the presence of certain modulators.

With respect to the composition, the fragrance component can be present in an amount of from about 0.04 wt % to 40 wt %, 1 wt % to about 30 wt %, about 5 wt % to about 30 wt %, or less than, equal to, or greater than about 0.04 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, or about 40 wt % relative to the composition.

Additionally with respect to the composition, the modulator can be present in an amount of from about 0.1 wt % to about 30 wt %, about 0.1 wt % to about 27 wt %, about 0.5 wt % to about 20 wt %, or less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 wt % relative to the composition.

As described herein, the fragrance imparting materials have been classified as low, moderate or high volatile fragrance materials according to their volatility by their vapor pressure. This method of classifying fragrance materials by their vapor pressure avoids the problem of different classifications for the same fragrance material according to the traditional approach that relies on their subjective characteristic character. In the case that the fragrance materials are a natural oil, extract or absolute, which comprises a mixture of several compounds, the vapor pressure of the complete oil should be treated a mixture of the individual perfume raw material components using the reference program cited above. The individual components and their level, in any given natural oil or extract, can be determined by direct injection of the oil into a GC-MS column for analysis as known by one skilled in the art. In the scenario that the fragrance materials are a proprietary specialty accord, so called ‘bases’, the vapor pressure, using the reference program cited above, should preferably be obtained from the supplier. However, it is understood by one skilled in the art that they can physically analyze the composition of a full fragrance oil available commercially to identity the fragrance raw materials and their levels using standard GC-MS techniques. This would be irrespective of whether they had been added to the fragrance oil as individual chemicals, as components of naturals or from proprietary bases. Although proprietary bases and naturals are included in our examples, when analyzing a commercially available fragrance via GC-MS one could simply identify the components of the base or natural oil as part of the overall fragrance mixture and their levels, without being able to identify which proprietary base or natural oil the fragrance had come from.

A pH of the composition can be in a range of from about 4 to about 8, about 5 to about 7, less than, equal to, or greater than about 4, 5, 6, 7, or about 8. The ethanol concentration in any of the compositions described herein can be less than about 78 wt % ethanol, less than about 70 wt % ethanol, less than about 60 wt % ethanol, less than about 50 wt % ethanol, less than about 40 wt % ethanol, less than about 30 wt % ethanol, less than about 20 wt % ethanol, less than about 10 wt % ethanol, or free of ethanol.

### Low Volatile Fragrance Materials

The fragrance component comprises at least one low volatile fragrance material. Individual low volatile fragrance materials or aggregate low volatile fragrance materials are those having a vapor pressure less than 0.001 Torr (0.000133 kPa) at 25° C. According to some examples, the composition can include at least 3 low volatile fragrance materials, or at least 4 low volatile fragrance materials, or at least 5 low volatile fragrance materials, or at least 7 low volatile fragrance materials.

If there are more than one low volatile fragrance materials, then the ranges provided hereinabove cover the total of all the low volatile fragrance materials. Examples of suitable low volatile fragrances materials are provided in Table 1A and 1B below.

Preferably, the low volatile fragrance material is selected from at least 1 material, or at least 2 materials, or at least 3 materials, or at least 5 materials, at least 7, at least 8, at least 10, or at least 12 low volatile fragrance materials as disclosed in Table 1A. Natural fragrance materials or oils having an aggregate vapor pressure less than 0.001 Torr (0.000133 kPa) at 25° C. are provided in Table 1B. Low Volatile Natural Oils.

TABLE 1A
Low Volatile Fragrance Materials

| | | | | Vapor |
|---|---|---|---|---|
| | | | Common | Pressure (Torr |
| No. | CAS Number | IUPAC Name | Name** | at 25° C.)* |
| 1. | 1211-29-6 | Cyclopentaneacetic acid, | Methyl | 0.00096500 |
| | | 3-oxo-2-(2Z)-2-penten-1- | jasmonate | |
| | | yl-, methyl ester, (1R,2R)- | | |
| 2. | 28219-60-5 | 2-Buten-1-ol, 2-methyl- | Hindinol | 0.00096100 |
| | | 4-(2,2,3-trimethyl-3- | | |
| | | cyclopenten-1-yl)- | | |
| 3. | 93-08-3 | Ethanone, 1-(2- | Methyl beta- | 0.00095700 |
| | | naphthalenyl)- | naphthyl ketone | |
| 4. | 67633-95-8 | 3-Decanone, 1-hydroxy- | Methyl Lavender | 0.00095100 |
| | | | Ketone | |
| 5. | 198404-98-7 | Cyclopropanemethanol, | Javanol ® | 0.00090200 |
| | | 1-methyl-2-[(1,2,2- | | |
| | | trimethylbicyclo[3.1.0]hex- | | |
| | | 3-yl)methyl]- | | |
| 6. | 121-32-4 | Benzaldehyde, 3-ethoxy- | Ethyl vanillin | 0.00088400 |
| | | 4-hydroxy- | | |
| 7. | 72403-67-9 | 3-Cyclohexene-1- | Myraldylacetate | 0.00087900 |
| | | methanol, 4-(4-methyl-3- | | |
| | | penten-1-yl)-, 1-acetate | | |
| 8. | 28940-11-6 | 2H-1,5-Benzodioxepin- | Calone | 0.00083100 |
| | | 3(4H)-one, 7-methyl- | | |
| 9. | 139504-68-0 | 2-Butanol, 1-[[2-(1,1- | Amber core | 0.00080300 |
| | | dimethylethyl)cyclohexyl]oxy]- | | |
| 10. | 502847-01-0 | Spiro[5.5]undec-8-en-1- | Spiro[5.5]undec- | 0.00073100 |
| | | one, 2,2,7,9-tetramethyl- | 8-en-1-one, | |
| | | | 2,2,7,9- | |
| | | | tetramethyl- | |
| 11. | 2570-03-8 | Cyclopentaneacetic acid, | trans-Hedione | 0.00071000 |
| | | 3-oxo-2-pentyl-, methyl | | |
| | | ester, (1R,2R)-rel- | | |
| 12. | 24851-98-7 | Cyclopentaneacetic acid, | Methyl | 0.00071000 |
| | | 3-oxo-2-pentyl-, methyl | dihydrojasmonate | |
| | | ester | or alternatives ¹ | |
| 13. | 101-86-0 | Octanal, 2- | Hexyl cinnamic | 0.00069700 |
| | | (phenylmethylene)- | aldehyde | |
| 14. | 365411-50-3 | Indeno[4,5-d]-1,3-dioxin, | Nebulone | 0.00069200 |
| | | 4,4a,5,6,7,8,9,9b- | | |
| | | octahydro-7,7,8,9,9- | | |
| | | pentamethyl- | | |
| 15. | 37172-53-5 | Cyclopentanecarboxylic | Dihydro Iso | 0.00067500 |
| | | acid, 2-hexyl-3-oxo-, | Jasmonate | |
| | | methyl ester | | |
| 16. | 65113-99-7 | 3-Cyclopentene-1- | Sandalore ® | 0.00062500 |
| | | butanol, α,β,2,2,3- | | |
| | | pentamethyl- | | |
| 17. | 68133-79-9 | Cyclopentanone, 2-(3,7- | Apritone | 0.00062000 |
| | | dimethyl-2,6-octadien-1-yl)- | | |
| 18. | 7212-44-4 | 1,6,10-Dodecatrien-3-ol, | Nerolidol | 0.00061600 |
| | | 3,7,11-trimethyl- | | |
| 19. | 53243-59-7 | 2-Pentenenitrile, 3- | Citronitril | 0.00061500 |
| | | methyl-5-phenyl-, (2Z)- | | |
| 20. | 134123-93-6 | Benzenepropanenitrile, | Fleuranil | 0.00057600 |
| | | 4-ethyl-α,α-dimethyl- | | |
| 21. | 77-53-2 | 1H-3a,7-Methanoazulen- | Cedrol Crude | 0.00056900 |
| | | 6-ol, octahydro-3,6,8,8- | | |
| | | tetramethyl-, | | |
| | | (3R,3aS,6R,7R,8aS)- | | |
| 22. | 68155-66-8 | Ethanone, 1- | Iso Gamma Super | 0.00056500 |
| | | (1,2,3,5,6,7,8,8a- | | |
| | | octahydro-2,3,8,8- | | |
| | | tetramethyl-2-naphthalenyl)- | | |
| 23. | 54464-57-2 | Ethanone, 1- | Iso-E Super ® | 0.00053800 |
| | | (1,2,3,4,5,6,7,8- | | |
| | | octahydro-2,3,8,8- | | |
| | | tetramethyl-2-naphthalenyl)- | | |
| 24. | 774-55-0 | Ethanone, 1-(5,6,7,8- | Florantone | 0.00053000 |
| | | tetrahydro-2-naphthalenyl)- | | |
| 25. | 141-92-4 | 2-Octanol, 8,8- | Hydroxycitronellal | 0.00052000 |
| | | dimethoxy-2,6-dimethyl- | Dimethyl Acetal | |
| 26. | 20665-85-4 | Propanoic acid, 2- | Vanillin | 0.00051200 |
| | | methyl-, 4-formyl-2- | isobutyrate | |
| | | methoxyphenyl ester | | |
| 27. | 79-78-7 | 1,6-Heptadien-3-one, 1- | Hexalon | 0.00049800 |
| | | (2,6,6-trimethyl-2- | | |
| | | cyclohexen-1-yl)- | | |
| 28. | 6259-76-3 | Benzoic acid, 2-hydroxy-, | Hexyl Salicylate | 0.00049100 |
| | | hexyl ester | | |
| 29. | 93-99-2 | Benzoic acid, phenyl ester | Phenyl Benzoate | 0.00047900 |
| 30. | 153859-23-5 | Cyclohexanepropanol, | Norlimbanol | 0.00046900 |
| | | 2,2,6-trimethyl-α-propyl-, | | |
| | | (1R,6S)- | | |
| 31. | 70788-30-6 | Cyclohexanepropanol, | Timberol/ | 0.00046900 |
| | | 2,2,6-trimethyl-α-propyl- | Norlimbanol | |
| 32. | 68555-58-8 | Benzoic acid, 2-hydroxy-, | Prenyl | 0.00045700 |
| | | 3-methyl-2-buten-1-yl ester | Salicylate | |
| 33. | 950919-28-5 | 2H-1,5-Benzodioxepin-3(4H)- | Cascalone | 0.00045500 |
| | | one, 7-(1-methylethyl)- | | |
| 34. | 30168-23-1 | Butanal, 4-(octahydro-4,7- | Dupical | 0.00044100 |
| | | methano-5H-inden-5-ylidene)- | | |
| 35. | 1222-05-5 | Cyclopenta[g]-2- | Galaxolide ® | 0.00041400 |
| | | benzopyran, 1,3,4,6,7,8- | | |
| | | hexahydro-4,6,6,7,8,8- | | |
| | | hexamethyl- | | |
| 36. | 4602-84-0 | 2,6,10-Dodecatrien-1-ol, | Farnesol | 0.00037000 |
| | | 3,7,11-trimethyl- | | |
| 37. | 95962-14-4 | Cyclopentanone, 2-[2-(4- | Nectaryl | 0.00036700 |
| | | methyl-3-cyclohexen-1- | | |
| | | yl)propyl]- | | |
| 38. | 4674-50-4 | 2(3H)-Naphthalenone, | Nootkatone | 0.00035800 |
| | | 4,4a,5,6,7,8-hexahydro- | | |
| | | 4,4a-dimethyl-6-(1- | | |
| | | methylethenyl)-, (4R,4aS,6R)- | | |
| 39. | 3487-99-8 | 2-Propenoic acid, 3- | Amyl Cinnamate | 0.00035200 |
| | | phenyl-, pentyl ester | | |
| 40. | 10522-41-5 | 2-hydroxy-2-phenylethyl | Styrolyl Acetate | 0.00033900 |
| | | acetate | | |
| 41. | 118-71-8 | 4H-Pyran-4-one, 3- | Maltol | 0.00033700 |
| | | hydroxy-2-methyl- | | |
| 42. | 128119-70-0 | 1-Propanol, 2-methyl-3- | Bornafix | 0.00033400 |
| | | [(1,7,7- | | |
| | | trimethylbicyclo[2.2.1]hept- | | |
| | | 2-yl)oxy]- | | |
| 43. | 103614-86-4 | 1-Naphthalenol, | Octalynol | 0.00033200 |
| | | 1,2,3,4,4a,5,8,8a- | | |
| | | octahydro-2,2,6,8- | | |
| | | tetramethyl- | | |
| 44. | 7785-33-3 | 2-Butenoic acid, 2- | Geranyl Tiglate | 0.00033200 |
| | | methyl-, (2E)-3,7- | | |
| | | dimethyl-2,6-octadien-1- | | |
| | | yl ester, (2E)- | | |
| 45. | 117933-89-8 | 1,3-Dioxane, 2-(2,4- | Karanal | 0.00033100 |
| | | dimethyl-3-cyclohexen- | | |
| | | 1-yl)-5-methyl-5-(1- | | |
| | | methylpropyl)- | | |
| 46. | 629-92-5 | Nonadecane | Nonadecane | 0.00032500 |
| 47. | 67801-20-1 | 4-Penten-2-ol, 3-methyl- | Ebanol | 0.00028100 |
| | | 5-(2,2,3-trimethyl-3- | | |
| | | cyclopenten-1-yl)- | | |
| 48. | 65416-14-0 | Propanoic acid, 2- | Maltol | 0.00028000 |
| | | methyl-, 2-methyl-4-oxo- | Isobutyrate | |
| | | 4H-pyran-3-yl ester | | |
| 49. | 28219-61-6 | 2-Buten-1-ol, 2-ethyl-4- | Laevo Trisandol | 0.00028000 |
| | | (2,2,3-trimethyl-3- | | |
| | | cyclopenten-1-yl)- | | |
| 50. | 5986-55-0 | 1,6-Methanonaphthalen- | Healingwood | 0.00027800 |
| | | 1(2H)-ol, octahydro- | | |
| | | 4,8a,9,9-tetramethyl-, | | |
| | | (1R,4S,4aS,6R,8aS)- | | |
| 51. | 195251-91-3 | 2H-1,5-Benzodioxepin- | Transluzone | 0.00026500 |
| | | 3(4H)-one, 7-(1,1- | | |
| | | dimethylethyl)- | | |
| 52. | 3100-36-5 | 8-Cyclohexadecen-1-one | Cyclohexadecenone | 0.00025300 |
| 53. | 65405-77-8 | Benzoic acid, 2-hydroxy-, | cis-3-Hexenyl | 0.00024600 |
| | | (3Z)-3-hexen-1-yl ester | salicylate | |
| 54. | 4940-11-8 | 4H-Pyran-4-one, 2-ethyl- | Ethyl Maltol | 0.00022800 |
| | | 3-hydroxy- | | |
| 55. | 541-91-3 | Cyclopentadecanone, 3- | Muskone | 0.00017600 |
| | | methyl- | | |
| 56. | 118-58-1 | Benzoic acid, 2-hydroxy-, | Benzyl salicylate | 0.00017500 |
| | | phenylmethyl ester | | |
| 57. | 81783-01-9 | 6,8-Nonadien-3-one, | Labienoxime | 0.00017300 |
| | | 2,4,4,7-tetramethyl-, oxime | | |
| 58. | 25485-88-5 | Benzoic acid, 2-hydroxy-, | Cyclohexyl | 0.00017300 |
| | | cyclohexyl ester | Salicylate | |
| 59. | 91-87-2 | Benzene, [2- | Amyl Cinnamic | 0.00016300 |
| | | (dimethoxymethyl)-1- | Aldehyde | |
| | | hepten-1-yl]- | Dimethyl Acetal | |
| 60. | 104864-90-6 | 3-Cyclopentene-1- | Firsantol | 0.00016000 |
| | | butanol, β,2,2,3- | | |
| | | tetramethyl-δ-methylene- | | |
| 61. | 224031-70-3 | 4-Penten-1-one, 1- | Spirogalbanone | 0.00015300 |
| | | spiro[4.5]dec-7-en-7-yl- | | |
| 62. | 134-28-1 | 5-Azulenemethanol, | Guaiyl Acetate | 0.00013400 |
| | | 1,2,3,4,5,6,7,8-octahydro- | | |
| | | α,α,3,8-tetramethyl-, | | |
| | | 5-acetate, (3S,5R,8S)- | | |
| 63. | 236391-76-7 | Acetic acid, 2-(1- | Romandolide ® | 0.00012400 |
| | | oxopropoxy)-, 1-(3,3- | | |
| | | dimethylcyclohexyl)ethyl ester | | |
| 64. | 115-71-9 | 2-Penten-1-ol, 5- | cis-alpha- | 0.00011800 |
| | | [(1R,3R,6S)-2,3- | Santalol | |
| | | dimethyltricyclo[2.2.1.02,6]hept- | | |
| | | 3-yl]-2-methyl-, (2Z)- | | |
| 65. | 107898-54-4 | 4-Penten-2-ol, 3,3-dimethyl- | Polysantol ® | 0.00011700 |
| | | 5-(2,2,3-trimethyl-3- | | |
| | | cyclopenten-1-yl)- | | |
| 66. | 69486-14-2 | 5,8-Methano-2H-1- | Florex ® | 0.00011000 |
| | | benzopyran-2-one, 6- | | |
| | | ethylideneoctahydro- | | |
| 67. | 84697-09-6 | Heptanal, 2-[(4- | Acalea | 0.00010100 |
| | | methylphenyl)methylene]- | | |
| 68. | 14595-54-1 | 4-Cyclopentadecen-1-one, (4Z)- | Exaltenone | 0.00009640 |
| 69. | 32388-55-9 | Ethanone, 1-[(3R,3aR,7R,8aS)- | Vertofix ® | 0.00008490 |
| | | 2,3,4,7,8,8a-hexahydro- | | |
| | | 3,6,8,8-tetramethyl-1H- | | |
| | | 3a, 7-methanoazulen-5- | | |
| | | yl]- | | |
| 70. | 131812-67-4 | 1,3-Dioxolane, 2,4- | Okoumal ® | 0.00007600 |
| | | dimethyl-2-(5,6,7,8- | | |
| | | tetrahydro-5,5,8,8- | | |
| | | tetramethyl-2- | | |
| | | naphthalenyl)- | | |
| 71. | 106-02-5 | Oxacyclohexadecan-2-one | Exaltolide ® | 0.00006430 |
| 72. | 141773-73-1 | 1-Propanol, 2-[1-(3,3- | Helvetolide ® | 0.00005790 |
| | | dimethylcyclohexyl)ethoxy]- | | |
| | | 2-methyl-, 1-propanoate | | |
| 73. | 63314-79-4 | 5-Cyclopentadecen-1- | Delta Muscenone | 0.00005650 |
| | | one, 3-methyl- | | |
| 74. | 77-42-9 | 2-Penten-1-ol, 2-methyl- | cis-beta- | 0.00004810 |
| | | 5-[(1S,2R,4R)-2-methyl-3- | Santalol | |
| | | methylenebicyclo[2.2.1]hept- | | |
| | | 2-yl]-, (2Z)- | | |
| 75. | 362467-67-2 | 2H-1,5-Benzodioxepin-3(4H)- | Azurone | 0.00004770 |
| | | one, 7-(3-methylbutyl)- | | |
| 76. | 28371-99-5 | Ethanone, 1-(2,6,10- | Trimofix O | 0.00004580 |
| | | trimethyl-2,5,9- | | |
| | | cyclododecatrien-1-yl)- | | |
| 77. | 16223-63-5 | 1H-3a,6-Methanoazulene-3- | Khusimol | 0.00004400 |
| | | methanol, octahydro-7,7- | | |
| | | dimethyl-8-methylene-, | | |
| | | (3S,3aR,6R,8aS)- | | |
| 78. | 10461-98-0 | Benzeneacetonitrile, α- | Peonile | 0.00004290 |
| | | cyclohexylidene- | | |
| 79. | 90-17-5 | Benzenemethanol, α- | Rosacetol | 0.00004240 |
| | | (trichloromethyl)-, 1-acetate | | |
| 80. | 50607-64-2 | Benzoic acid, 2-[(2- | Mevantraal | 0.00004070 |
| | | methylpentylidene)amino]-, | | |
| | | methyl ester | | |
| 81. | 29895-73-6 | 5-Hydroxy-2-benzyl-1,3- | Acetal CD | 0.00004050 |
| | | dioxane | | |
| 82. | 94-47-3 | Benzoic acid, 2- | Phenyl Ethyl | 0.00003480 |
| | | phenylethyl ester | Benzoate | |
| 83. | 3100-36-5 | Cyclohexadec-8-en-1-one | Globanone ® | 0.00003310 |
| 84. | 37609-25-9 | 5-Cyclohexadecen-1-One | Ambretone | 0.00003310 |
| 85. | 66072-32-0 | Cyclohexanol, 4-(1,7,7- | Iso Bornyl | 0.00003010 |
| | | trimethylbicyclo[2.2.1]hept- | Cyclohexanol | |
| | | 2-yl)- | | |
| 86. | 31906-04-4 | 3-Cyclohexene-1- | Lyral ® | 0.00002940 |
| | | carboxaldehyde, 4-(4- | | |
| | | hydroxy-4-methylpentyl)- | | |
| 87. | 21145-77-7 | Ethanone, 1-(5,6,7,8- | Musk Plus | 0.00002860 |
| | | tetrahydro-3,5,5,6,8,8- | | |
| | | hexamethyl-2-naphthalenyl)- | | |
| 88. | 21145-77-7 | Ethanone, 1-(5,6,7,8- | Fixolide | 0.00002860 |
| | | tetrahydro-3,5,5,6,8,8- | | |
| | | hexamethyl-2-naphthalenyl)- | | |
| 89. | 22442-01-9 | 2-Cyclopentadecen-1- | Muscenone | 0.00002770 |
| | | one, 3-methyl- | | |
| 90. | 109-29-5 | Oxacycloheptadecan-2-one | Silvanone Ci | 0.00002600 |
| 91. | 101-94-0 | Benzeneacetic acid, 4- | Para Cresyl | 0.00002330 |
| | | methylphenyl ester | Phenyl Acetate | |
| 92. | 102-20-5 | Benzeneacetic acid, 2- | Phenyl Ethyl | 0.00002300 |
| | | phenylethyl ester | Phenyl Acetate | |
| 93. | 118562-73-5 | Cyclododecaneethanol, | Hydroxyambran | 0.00001800 |
| | | β-methyl- | | |
| 94. | 103-41-3 | 2-Propenoic acid, 3- | Benzyl | 0.00001050 |
| | | phenyl-, phenylmethyl ester | Cinnamate | |
| 95. | 4707-47-5 | Benzoic acid, 2,4- | Veramoss/ | 0.00001050 |
| | | dihydroxy-3,6-dimethyl-, | LRG201/Evernyl | |
| | | methyl ester | | |
| 96. | 183551-83-9 | Naphtho[2,1-b]furan- | Myrrhone | 0.00000977 |
| | | 6(7H)-one, 8,9-dihydro- | | |
| | | 1,5,8-trimethyl-, (8R)- | | |
| 97. | 102-17-0 | Benzeneacetic acid, (4- | Para Anisyl | 0.00000813 |
| | | methoxyphenyl)methyl ester | Phenyl Acetate | |
| 98. | 120-11-6 | Benzene, 2-methoxy-1- | Benzyl Iso | 0.00000676 |
| | | (phenylmethoxy)-4-(1- | Eugenol | |
| | | propen-1-yl)- | | |
| 99. | 102-22-7 | Benzeneacetic acid, (2E)- | Geranyl | 0.00000645 |
| | | 3,7-dimethyl-2,6- | Phenylacetate | |
| | | octadien-1-yl ester | | |
| 100. | 111879-80-2 | Oxacyclohexadec-12-en- | Habanolide 100% | 0.00000431 |
| | | 2-one, (12E)- | | |
| 101. | 87-22-9 | Benzoic acid, 2-hydroxy-, | Phenyl Ethyl | 0.00000299 |
| | | 2-phenylethyl ester | Salicylate | |
| 102. | 78-37-5 | 2-Propenoic acid, 3- | Linalyl | 0.00000174 |
| | | phenyl-, 1-ethenyl-1,5- | Cinnamate | |
| | | dimethyl-4-hexen-1-yl ester | | |
| 103. | 28645-51-4 | Oxacycloheptadec-10-en- | Ambrettolide | 0.00000139 |
| | | 2-one | | |
| 104. | 123-69-3 | Oxacycloheptadec-8-en- | Ambrettolide | 0.00000136 |
| | | 2-one, (8Z)- | | |
| 105. | 3391-83-1 | 1,7- | Musk RI | 0.00000057 |
| | | Dioxacycloheptadecan-8-one | | |
| 106. | 68527-79-7 | 7-Octen-2-ol, 8-(1H- | Indolene | 0.000000445 |
| | | indol-1-yl)-2,6-dimethyl- | | |
| 107. | 89-43-0 | Methyl 2-[(7-hydroxy-3,7- | Aurantinol | 0.0000000100 |
| | | dimethyloctylidene)amino]benzoate | | |
| 108. | 54982-83-1 | 1,4- | Zenolide | 0.00000000834 |
| | | Dioxacyclohexadecane-5,16-dione | | |
| 109. | 105-95-3 | 1,4- | Ethylene | 0.00000000313 |
| | | Dioxacycloheptadecane-5,17-dione | Brassylate | |
| 110. | 3681-73-0 | Hexadecanoic acid, (2E)- | Hexarose | 0.00000000300 |
| | | 3,7-dimethyl-2,6- | | |
| | | octadien-1-yl ester | | |
| 111. | 4159-29-9 | Phenol, 4-[3- | Coniferyl | 0.00000000170 |
| | | (benzoyloxy)-1-propen- | benzoate | |
| | | 1-yl]-2-methoxy- | | |
| 112. | 144761-91-1 | Benzoic acid, 2-[(1- | Trifone DIPG | 0.00000000093 |
| | | hydroxy-3- | | |
| | | phenylbutyl)amino]-, | | |
| | | methyl ester | | |

¹ Non-limiting examples of alternative qualities from various suppliers can be purchased under the following tradenames: Kharismal ® Super (IFF), Kharismal ® (IFF), Hedione ® (Firmenich), Hedione ® HC (Firmenich), Paradisone (Firmenich), Cepionate (Zenon), Super cepionate (Zenon), Claigeon ® (Zenon).
*Vapor Pressures are acquired as described in the Test Methods Section.
**Origin: The low volatile fragrance materials may be obtained from one or more of the following companies: Firmenich (Geneva, Switzerland), Symrise AG (Holzminden, Germany), Givaudan (Argenteuil, France), IFF (Hazlet, New Jersey), Bedoukian (Danbury, Connecticut), Sigma Aldrich (St. Louis, Missouri), Millennium Specialty Chemicals (Olympia Fields, Illinois), Polarone International (Jersey City, New Jersey), and Aroma & Flavor Specialties (Danbury, Connecticut).

TABLE 1B
Low Volatile Natural Oils.

| No. | Natural oil | Supplier |
|---|---|---|
| 1. | Beeswax Absolute | Robertet |
| 2. | Cedarwood Sawdust SFE | Firmenich |
| 3. | Cedarwood Oil Rect | Firmenich |
| 4. | Cedarwood Texas Light | H. Reynaud & Fils |
| 5. | Ciste Absolute | IFF |
| 6. | Cocoa Colorless Oil | Robertet |
| 7. | Cypriol Coeur Essence | Robertet |
| 8. | Guaiacwood Oil | Global Essence Inc |
| 9. | Incense Wood Natural | Robertet |
| 10. | Orris CO2 Extract | Mane |
| 11. | Patchouli Oil | IFF |
| 12. | Tolu Baume Res | Robertet |
| 13. | Vanilla Absolute | Robertet |
| 14. | Vanilla CO2 Absolute | Robertet |
| 15. | Vetivert Oil | IFF |
| 16. | Vetyvert Acetate | Robertet |

Suppliers:
Firmenich, Geneva, Switzerland
Global Essence Inc, New Jersey, USA
H. Reynaud & Fils, Montbrun-les-Bains, France
IFF, Hazlet, New Jersey, USA
Mane, Le Bar-sur-Loup, France
Robertet, Grasse, France

Exemplary low volatile fragrance materials selected from the group of Tables 1A or 1B Low Volatile Fragrance Materials are preferred. However, it is understood by one skilled in the art that other low volatile fragrance materials, not recited in Tables 1A or 1, would also fall within the scope of the present invention, so long as they have a vapor pressure less than 0.001 Torr (0.000133 kPa) at 25° C.

### (ii) Moderate Volatile Fragrance Materials

The fragrance component includes at least one moderate volatile fragrance material or aggregate of volatile fragrance materials having a vapor pressure in the range of 0.1 Torr to 0.001 Torr (0.0133 kPa to 0.000133 kPa) at 25° C. In some examples, the composition according to the present disclosure can include at least 3 moderate volatile fragrance materials, or at least 5 moderate volatile fragrance materials, or at least 7 moderate volatile fragrance materials. If there are more than one moderate volatile fragrance materials, then the ranges provided hereinabove cover the total of all of the moderate volatile fragrance materials. Suitable examples of moderate volatile fragrances materials are provided in Table 2A and 2B below.

Preferable examples of moderate volatile fragrance materials having a vapor pressure in the range of 0.1 Torr to 0.001 Torr (0.0133 kPa to 0.000133 kPa) at 25° C. are provided in Table 2A and 2B. Preferably, the moderate volatile fragrance material is selected from at least 1 material, or at least 2 materials, or at least 3 materials, or at least 5 materials, or at least 7 moderate volatile fragrance materials as disclosed in Table 2A. Natural fragrance materials or oils having an aggregate vapour pressure between 0.1 Torr to 0.001 Torr (0.0133 kPa to 0.000133 kPa) at 25° C. are provided in Table 2B. Moderate Volatile Natural Oils.

TABLE 2A
Moderate Volatile Fragrance Materials

| | | | | Vapor Pressure |
|---|---|---|---|---|
| No. | CAS Number | IUPAC Name | Common Name** | (Torr at 25° C.)* |
| 1. | 24168-70-5 | Pyrazine, 2- | Methoxyisobutylpyrazine | 0.09950000 |
| | | methoxy-3-(1- | | |
| | | methylpropyl)- | | |
| 2. | 89-79-2 | Cyclohexanol, 5- | Iso-Pulegol | 0.09930000 |
| | | methyl-2-(1- | | |
| | | methylethenyl)-, | | |
| | | (1R,2S,5R)- | | |
| 3. | 112-12-9 | 2-Undecanone | Methyl Nonyl | 0.09780000 |
| | | | Ketone | |
| 4. | 103-05-9 | Benzenepropanol, | Phenyl Ethyl | 0.09770000 |
| | | α,α-dimethyl- | Dimethyl | |
| | | | Carbinol | |
| 5. | 125-12-2 | Bicyclo[2.2.1]heptan- | Iso Bornyl | 0.09590000 |
| | | 2-ol, 1,7,7- | Acetate | |
| | | trimethyl-, 2-acetate, | | |
| | | (1R,2R,4R)-rel- | | |
| 6. | 78-70-6 | 1,6-Octadien-3-ol, | Linalool | 0.09050000 |
| | | 3,7-dimethyl- | | |
| 7. | 101-97-3 | Benzeneacetic acid, | Ethyl Phenyl | 0.08970000 |
| | | ethyl ester | Acetate | |
| 8. | 100-86-7 | Benzeneethanol, | Dimethyl Benzyl | 0.08880000 |
| | | α,α-dimethyl- | Carbinol | |
| 9. | 188570-78-7 | Cyclopropanecarboxylic | Montaverdi | 0.08640000 |
| | | acid, (3Z)-3- | | |
| | | hexen-1-yl ester | | |
| 10. | 67634-25-7 | 3-Cyclohexene-1- | Floralate | 0.08500000 |
| | | methanol, 3,5- | | |
| | | dimethyl-, 1-acetate | | |
| 11. | 112-44-7 | Undecanal | Undecyl | 0.08320000 |
| | | | Aldehyde | |
| 12. | 32669-00-4 | Ethanone, 1-(3- | Tanaisone ® | 0.08150000 |
| | | cycloocten-1-yl)- | | |
| 13. | 98-53-3 | Cyclohexanone, 4- | Patchi | 0.07780000 |
| | | (1,1-dimethylethyl)- | | |
| 14. | 35854-86-5 | 6-Nonen-1-ol, (6Z)- | cis-6-None-1-ol | 0.07770000 |
| 15. | 5331-14-6 | Benzene, (2- | Butyl phenethyl | 0.07760000 |
| | | butoxyethyl)- | ether | |
| 16. | 80-57-9 | Bicyclo[3.1.1]hept- | Verbenone | 0.07730000 |
| | | 3-en-2-one, 4,6,6- | | |
| | | trimethyl- | | |
| 17. | 22471-55-2 | Cyclohexanecarboxylic | Thesaron | 0.07670000 |
| | | acid, 2,2,6- | | |
| | | trimethyl-, ethyl | | |
| | | ester, (1R,6S)-rel- | | |
| 18. | 60-12-8 | Benzeneethanol | Phenethyl alcohol | 0.07410000 |
| 19. | 106-26-3 | 2,6-Octadienal, 3,7- | Neral | 0.07120000 |
| | | dimethyl-, (2Z)- | | |
| 20. | 5392-40-5 | 2,6-Octadienal, 3,7- | Citral | 0.07120000 |
| | | dimethyl- | | |
| 21. | 89-48-5 | Cyclohexanol, 5- | Menthyl Acetate | 0.07070000 |
| | | methyl-2-(1- | | |
| | | methylethyl)-, 1- | | |
| | | acetate, (1R,2S,5R)- | | |
| | | rel- | | |
| 22. | 119-36-8 | Benzoic acid, 2- | Methyl salicylate | 0.07000000 |
| | | hydroxy-, methyl | | |
| | | ester | | |
| 23. | 104-46-1 | Benzene, 1- | Anethol | 0.06870000 |
| | | methoxy-4-(1E)-1- | | |
| | | propen-1-yl- | | |
| 24. | 7549-37-3 | 2,6-Octadiene, 1,1- | Citral Dimethyl | 0.06780000 |
| | | dimethoxy-3,7- | Acetal | |
| | | dimethyl- | | |
| 25. | 25225-08-5 | Cyclohexanemethanol, | Aphermate | 0.06780000 |
| | | α,3,3-trimethyl-, | | |
| | | 1-formate | | |
| 26. | 3913-81-3 | 2-Decenal, (2E)- | 2-Decene-1-al | 0.06740000 |
| 27. | 15373-31-6 | 3-Cyclopentene-1- | Cantryl ® | 0.06700000 |
| | | acetonitrile, 2,2,3- | | |
| | | trimethyl- | | |
| 28. | 6485-40-1 | 2-Cyclohexen-1-one, | Laevo carvone | 0.06560000 |
| | | 2-methyl-5-(1- | | |
| | | methylethenyl)-, | | |
| | | (5R)- | | |
| 29. | 16587-71-6 | Cyclohexanone, 4- | Orivone | 0.06490000 |
| | | (1,1- | | |
| | | dimethylpropyl)- | | |
| 30. | 62406-73-9 | 6,10- | Opalal CI | 0.06290000 |
| | | Dioxaspiro[4.5]decane, | | |
| | | 8,8-dimethyl-7- | | |
| | | (1-methylethyl)- | | |
| 31. | 3720-16-9 | 2-Cyclohexen-1-one, | Livescone | 0.06270000 |
| | | 3-methyl-5-propyl- | | |
| 32. | 13816-33-6 | Benzonitrile, 4-(1- | Cumin Nitrile | 0.06230000 |
| | | methylethyl)- | | |
| 33. | 67019-89-0 | 2,6-Nonadienenitrile | Violet Nitrile | 0.06200000 |
| 34. | 53398-85-9 | Butanoic acid, 2- | cis-3-Hexenyl | 0.06130000 |
| | | methyl-, (3Z)-3- | Alpha Methyl | |
| | | hexen-1-yl ester | Butyrate | |
| 35. | 208041-98-9 | n/a | Jasmonitrile | 0.05920000 |
| 36. | 16510-27-3 | Benzene, 1- | Toscanol | 0.05870000 |
| | | (cyclopropylmethyl)- | | |
| | | 4-methoxy- | | |
| 37. | 111-80-8 | 2-Nonynoic acid, | Methyl Octine | 0.05680000 |
| | | methyl ester | Carbonate | |
| 38. | 103-45-7 | Acetic acid, 2- | Phenyl Ethyl | 0.05640000 |
| | | phenylethyl ester | Acetate | |
| 39. | 2550-26-7 | 2-Butanone, 4- | Benzyl Acetone | 0.05570000 |
| | | phenyl- | | |
| 40. | 13491-79-7 | Cyclohexanol, 2- | Verdol | 0.05430000 |
| | | (1,1-dimethylethyl)- | | |
| 41. | 7786-44-9 | 2,6-Nonadien-1-ol | 2,6-Nonadien-1- | 0.05370000 |
| | | | ol | |
| 42. | 103-28-6 | Propanoic acid, 2- | Benzyl Iso | 0.05130000 |
| | | methyl-, | Butyrate | |
| | | phenylmethyl ester | | |
| 43. | 104-62-1 | Formic acid, 2- | Phenyl Ethyl | 0.05050000 |
| | | phenylethyl ester | Formate | |
| 44. | 28462-85-3 | Bicyclo[2.2.1]heptan- | Humus Ether | 0.04870000 |
| | | 2-ol, 1,2,3,3- | | |
| | | tetramethyl-, | | |
| | | (1R,2R,4S)-rel- | | |
| 45. | 122-03-2 | Benzaldehyde, 4-(1- | Cuminic | 0.04820000 |
| | | methylethyl)- | Aldehyde | |
| 46. | 358331-95-0 | 2,5-Octadien-4-one, | Pomarose | 0.04810000 |
| | | 5,6,7-trimethyl-, | | |
| | | (2E)- | | |
| 47. | 562-74-3 | 3-Cyclohexen-1-ol, | Terpinenol-4 | 0.04780000 |
| | | 4-methyl-1-(1- | | |
| | | methylethyl)- | | |
| 48. | 68527-77-5 | 3-Cyclohexene-1- | Isocyclogeraniol | 0.04640000 |
| | | methanol, 2,4,6- | | |
| | | trimethyl- | | |
| 49. | 35852-46-1 | Pentanoic acid, (3Z)- | Cis-3-Hexenyl | 0.04580000 |
| | | 3-hexen-1-yl ester | Valerate | |
| 50. | 2756-56-1 | Bicyclo[2.2.1]heptan- | Iso Bornyl | 0.04540000 |
| | | 2-ol, 1,7,7- | Propionate | |
| | | trimethyl-, 2- | | |
| | | propanoate, | | |
| | | (1R,2R,4R)-rel- | | |
| 51. | 14374-92-6 | Benzene, 1-methyl- | Verdoracine | 0.04460000 |
| | | 4-(1-methylethyl)-2- | | |
| | | (1-propen-1-yl)- | | |
| 52. | 6784-13-0 | 3-Cyclohexene-1- | Limonenal | 0.04380000 |
| | | propanal, β,4- | | |
| | | dimethyl- | | |
| 53. | 8000-41-7 | 2-(4-methyl-1- | Alpha Terpineol | 0.04320000 |
| | | cyclohex-3- | | |
| | | enyl)propan-2-ol | | |
| 54. | 41884-28-0 | 1-Hexanol, 5- | Tetrahydro | 0.04230000 |
| | | methyl-2-(1- | Lavandulol | |
| | | methylethyl)-, (2R)- | | |
| 55. | 22457-23-4 | 3-Heptanone, 5- | Stemone ® | 0.04140000 |
| | | methyl-, oxime | | |
| 56. | 104-50-7 | 2(3H)-Furanone, 5- | Gamma | 0.04080000 |
| | | butyldihydro- | Octalactone | |
| 57. | 143-08-8 | 1-Nonanol | Nonyl Alcohol | 0.04070000 |
| 58. | 3613-30-7 | Octanal, 7-methoxy- | Methoxycitronellal | 0.04020000 |
| | | 3,7-dimethyl- | | |
| 59. | 67634-00-8 | Acetic acid, 2-(3- | Allyl Amyl | 0.04000000 |
| | | methylbutoxy)-, 2- | Glycolate | |
| | | propen-1-yl ester | | |
| 60. | 464-45-9 | Bicyclo[2.2.1]heptan- | l-Borneol | 0.03980000 |
| | | 2-ol, 1,7,7- | | |
| | | trimethyl-, | | |
| | | (1S,2R,4S)- | | |
| 61. | 124-76-5 | Bicyclo[2.2.1]heptan- | 1.7.7-Trimethyl- | 0.03980000 |
| | | 2-ol, 1,7,7- | Bicyclo-1.2.2- | |
| | | trimethyl-, | Heptanol-2 | |
| | | (1R,2R,4R)-rel- | | |
| 62. | 67874-72-0 | Cyclohexanol, 2- | Coniferan | 0.03980000 |
| | | (1,1- | | |
| | | dimethylpropyl)-, | | |
| | | 1-acetate | | |
| 63. | 80-26-2 | 3-Cyclohexene-1- | Terpinyl Acetate | 0.03920000 |
| | | methanol, α,α,4- | | |
| | | trimethyl-, 1-acetate | | |
| 64. | 498-81-7 | Cyclohexanemethanol, | Dihydro | 0.03920000 |
| | | α,α,4-trimethyl- | Terpineol | |
| 65. | 112-45-8 | 10-Undecenal | Undecylenic | 0.03900000 |
| | | | aldehyde | |
| 66. | 35044-57-6 | 2,4-Cyclohexadiene- | Ethyl Safranate | 0.03880000 |
| | | 1-carboxylic acid, | | |
| | | 2,6,6-trimethyl-, | | |
| | | ethyl ester | | |
| 67. | 106-21-8 | 1-Octanol, 3,7- | Dimethyl Octanol | 0.03860000 |
| | | dimethyl- | | |
| 68. | 84560-00-9 | Cyclopentanol, 2- | Cyclopentol | 0.03790000 |
| | | pentyl- | | |
| 69. | 82461-14-1 | Furan, tetrahydro- | Rhubafuran ® | 0.03780000 |
| | | 2,4-dimethyl-4- | | |
| | | phenyl- | | |
| 70. | 56011-02-0 | Benzene, [2-(3- | Phenyl Ethyl | 0.03690000 |
| | | methylbutoxy)ethyl]- | Isoamyl Ether | |
| 71. | 103-37-7 | Butanoic acid, | Benzyl Butyrate | 0.03660000 |
| | | phenylmethyl ester | | |
| 72. | 6378-65-0 | Hexyl hexanoate | Hexyl hexanoate | 0.03490000 |
| 73. | 118-61-6 | Benzoic acid, 2- | Ethyl salicylate | 0.03480000 |
| | | hydroxy-, ethyl ester | | |
| 74. | 98-52-2 | Cyclohexanol, 4- | Patchon | 0.03480000 |
| | | (1,1-dimethylethyl)- | | |
| 75. | 115-99-1 | 1,6-Octadien-3-ol, | Linalyl Formate | 0.03440000 |
| | | 3,7-dimethyl-, 3- | | |
| | | formate | | |
| 76. | 112-54-9 | Dodecanal | Lauric Aldehyde | 0.03440000 |
| 77. | 53046-97-2 | 3,6-Nonadien-1-ol, | 3,6 Nonadien-1-ol | 0.03360000 |
| | | (3Z,6Z)- | | |
| 78. | 76649-25-7 | 3,6-Nonadien-1-ol | 3,6-Nonadien-1- | 0.03360000 |
| | | | ol | |
| 79. | 141-25-3 | 3,7-Dimethyloct-6- | Rhodinol | 0.03290000 |
| | | en-1-ol | | |
| 80. | 1975-78-6 | Decanenitrile | Decanonitrile | 0.03250000 |
| 81. | 2216-51-5 | Cyclohexanol, 5- | L-Menthol | 0.03230000 |
| | | methyl-2-(1- | | |
| | | methylethyl)-, | | |
| | | (1R,2S,5R)- | | |
| 82. | 3658-77-3 | 4-hydroxy-2,5- | Pineapple Ketone | 0.03200000 |
| | | dimethylfuran-3-one | | |
| 83. | 103-93-5 | Propanoic acid, 2- | Para Cresyl iso- | 0.03120000 |
| | | methyl-, 4- | Butyrate | |
| | | methylphenyl ester | | |
| 84. | 24717-86-0 | Propanoic acid, 2- | Abierate | 0.03110000 |
| | | methyl-, (1R,2S,4R)- | | |
| | | 1,7,7-trimeth- | | |
| | | ylbicyclo[2.2.1]hept- | | |
| | | 2-yl ester, rel- | | |
| 85. | 67845-46-9 | Acetaldehyde, 2-(4- | Aldehyde XI | 0.03090000 |
| | | methylphenoxy)- | | |
| 86. | 67883-79-8 | 2-Butenoic acid, 2- | Cis-3-Hexenyl | 0.03060000 |
| | | methyl-, (3Z)-3- | Tiglate | |
| | | hexen-1-yl ester, | | |
| | | (2E)- | | |
| 87. | 33885-51-7 | Bicyclo[3.1.1]hept- | Pino | 0.03040000 |
| | | 2-ene-2-propanal, | Acetaldehyde | |
| | | 6,6-dimethyl- | | |
| 88. | 105-85-1 | 6-Octen-1-ol, 3,7- | Citronellyl | 0.03000000 |
| | | dimethyl-, 1-formate | Formate | |
| 89. | 70214-77-6 | 2-Nonanol, 6,8- | Nonadyl | 0.03010000 |
| | | dimethyl- | | |
| 90. | 215231-33-7 | Cyclohexanol, 1- | Rossitol | 0.02990000 |
| | | methyl-3-(2- | | |
| | | methylpropyl)- | | |
| 91. | 120-72-9 | 1H-Indole | Indole | 0.02980000 |
| 92. | 2463-77-6 | 2-Undecenal | 2-Undecene-1-al | 0.02970000 |
| 93. | 675-09-2 | 2H-Pyran-2-one, | Levistamel | 0.02940000 |
| | | 4,6-dimethyl- | | |
| 94. | 98-55-5 | 3-Cyclohexene-1- | Alpha-Terpineol | 0.02830000 |
| | | methanol, α,α,4- | | |
| | | trimethyl- | | |
| 95. | 81786-73-4 | 3-Hepten-2-one, | Koavone | 0.02750000 |
| | | 3,4,5,6,6- | | |
| | | pentamethyl-, (3Z)- | | |
| 96. | 122-97-4 | Benzenepropanol | Phenyl Propyl | 0.02710000 |
| | | | Alcohol | |
| 97. | 39212-23-2 | 2(3H)-Furanone, 5- | Methyl | 0.02700000 |
| | | butyldihydro-4- | Octalactone | |
| | | methyl- | | |
| 98. | 53767-93-4 | 7-Octen-2-ol, 2,6- | Dihydro Terpinyl | 0.02690000 |
| | | dimethyl-, 2-acetate | Acetate | |
| 99. | 35044-59-8 | 1,3-Cyclohexadiene- | Ethyl Safranate | 0.02660000 |
| | | 1-carboxylic acid, | | |
| | | 2,6,6-trimethyl-, | | |
| | | ethyl ester | | |
| 100. | 104-55-2 | 2-Propenal, 3- | Cinnamic | 0.02650000 |
| | | phenyl- | Aldehyde | |
| 101. | 144-39-8 | 1,6-Octadien-3-ol, | Linalyl | 0.02630000 |
| | | 3,7-dimethyl-, 3- | Propionate | |
| | | propanoate | | |
| 102. | 61931-80-4 | 1,6-Nonadien-3-ol, | 3,7-Dimethyl-1,6- | 0.02630000 |
| | | 3,7-dimethyl-, 3- | nonadien-3-yl | |
| | | acetate | acetate | |
| 103. | 102-13-6 | Benzeneacetic acid, | Iso Butyl | 0.02630000 |
| | | 2-methylpropyl ester | Phenylacetate | |
| 104. | 65443-14-3 | Cyclopentanone, | Veloutone | 0.02610000 |
| | | 2,2,5-trimethyl-5- | | |
| | | pentyl- | | |
| 105. | 141-12-8 | 2,6-Octadien-1-ol, | Neryl Acetate | 0.02560000 |
| | | 3,7-dimethyl-, 1- | | |
| | | acetate, (2Z)- | | |
| 106. | 105-87-3 | 2,6-Octadien-1-ol, | Geranyl acetate | 0.02560000 |
| | | 3,7-dimethyl-, 1- | | |
| | | acetate, (2E)- | | |
| 107. | 68141-17-3 | Undecane, 1,1- | Methyl Nonyl | 0.02550000 |
| | | dimethoxy-2- | Acetaldehyde | |
| | | methyl- | Dimethyl Acetal | |
| 108. | 2206-94-2 | Benzenemethanol, | Indocolore | 0.02550000 |
| | | α-methylene-, 1- | | |
| | | acetate | | |
| 109. | 10528-67-3 | Cyclohexanepropanol, | Cyclohexylmagnol | 0.02550000 |
| | | α-methyl- | | |
| 110. | 123-11-5 | Benzaldehyde, 4- | Anisic Aldehyde | 0.02490000 |
| | | methoxy- | | |
| 111. | 57576-09-7 | Cyclohexanol, 5- | Iso Pulegol | 0.02480000 |
| | | methyl-2-(1- | Acetate | |
| | | methylethenyl)-, 1- | | |
| | | acetate, (1R,2S,5R)- | | |
| 112. | 51566-62-2 | 6-Octenenitrile, 3,7- | Citronellyl Nitrile | 0.02470000 |
| | | dimethyl- | | |
| 113. | 60335-71-9 | 2H-Pyran, 3,6- | Rosyrane Super | 0.02470000 |
| | | dihydro-4-methyl-2- | | |
| | | phenyl- | | |
| 114. | 30385-25-2 | 6-Octen-2-ol, 2,6- | Dihydromyrcenol | 0.02440000 |
| | | dimethyl- | | |
| 115. | 101-84-8 | Benzene, 1,1′- | Diphenyl Oxide | 0.02230000 |
| | | oxybis- | | |
| 116. | 136-60-7 | Benzoic acid, butyl | Butyl Benzoate | 0.02170000 |
| | | ester | | |
| 117. | 93939-86-7 | 5,8-Methano-2H-1- | Rhuboflor | 0.02120000 |
| | | benzopyran, 6- | | |
| | | ethylideneoctahydro- | | |
| 118. | 83926-73-2 | Cyclohexanepropanol, | Coranol | 0.02100000 |
| | | α,α-dimethyl- | | |
| 119. | 125109-85-5 | Benzenepropanal, β- | Florhydral | 0.02070000 |
| | | methyl-3-(1- | | |
| | | methylethyl)- | | |
| 120. | 104-21-2 | Benzenemethanol, | Anisyl Acetate | 0.02050000 |
| | | 4-methoxy-, 1- | | |
| | | acetate | | |
| 121. | 1365-19-1 | 2-Furanmethanol, 5- | Linalool Oxide | 0.02050000 |
| | | ethenyltetrahydro- | | |
| | | α,α,5-trimethyl- | | |
| 122. | 137-03-1 | Cyclopentanone, 2- | Frutalone | 0.02040000 |
| | | heptyl- | | |
| 123. | 2563-07-7 | Phenol, 2-ethoxy-4- | Ultravanil | 0.02030000 |
| | | methyl- | | |
| 124. | 1128-08-1 | 2-Cyclopenten-1- | Dihydrojasmone | 0.02020000 |
| | | one, 3-methyl-2- | | |
| | | pentyl- | | |
| 125. | 7493-57-4 | Benzene, [2-(1- | Acetaldehyde | 0.01990000 |
| | | propoxyethoxy)ethyl]- | | |
| 126. | 141-25-3 | 7-Octen-1-ol, 3,7- | Rhodinol | 0.01970000 |
| | | dimethyl- | | |
| 127. | 216970-21-7 | Bicyclo[4.3.1]decane, | 3-Methoxy-7,7- | 0.01960000 |
| | | 3-methoxy-7,7- | dimethyl-10- | |
| | | dimethyl-10- | methylenebicyclo | |
| | | methylene- | [4.3.1]decane | |
| 128. | 319002-92-1 | Propanoic acid, 2- | Sclareolate ® | 0.01960000 |
| | | (1,1- | | |
| | | dimethylpropoxy)-, | | |
| | | propyl ester, (2S)- | | |
| 129. | 85-91-6 | Benzoic acid, 2- | Dimethyl | 0.01930000 |
| | | (methylamino)-, | anthranilate | |
| | | methyl | | |
| 130. | 13828-37-0 | Cyclohexanemethanol, | Mayol | 0.01920000 |
| | | 4-(1- | | |
| | | methylethyl)-, cis- | | |
| 131. | 26330-65-4 | (E)-6-ethyl-3- | Super Muguet | 0.01850000 |
| | | methyloct-6-en-1-ol | | |
| 132. | 7540-51-4 | 6-Octen-1-ol, 3,7- | L-Citronellol | 0.01830000 |
| | | dimethyl-, (3S)- | | |
| 133. | 106-22-9 | 6-Octen-1-ol, 3,7- | Citronellol | 0.01830000 |
| | | dimethyl- | | |
| 134. | 543-39-5 | 7-Octen-2-ol, 2- | Myrcenol | 0.01820000 |
| | | methyl-6-methylene- | | |
| 135. | 7775-00-0 | Benzenepropanal, 4- | Cyclemax | 0.01820000 |
| | | (1-methylethyl)- | | |
| 136. | 18479-54-4 | 4,6-Octadien-3-ol, | Muguol | 0.01800000 |
| | | 3,7-dimethyl- | | |
| 137. | 29214-60-6 | Octanoic acid, 2- | Gelsone | 0.01790000 |
| | | acetyl-, ethyl ester | | |
| 138. | 1209-61-6 | 5- | Tobacarol | 0.01730000 |
| | | Oxatricyclo[8.2.0.04,6]do- | | |
| | | decane, | | |
| | | 4,9,12,12- | | |
| | | tetramethyl- | | |
| 139. | 57934-97-1 | 2-Cyclohexene-1- | Givescone | 0.01710000 |
| | | carboxylic acid, 2- | | |
| | | ethyl-6,6-dimethyl-, | | |
| | | ethyl ester | | |
| 140. | 14901-07-6 | 3-Buten-2-one, 4- | Beta-Ionone | 0.01690000 |
| | | (2,6,6-trimethyl-1- | | |
| | | cyclohexen-1-yl)-, | | |
| | | (3E)- | | |
| 141. | 64001-15-6 | 4,7-Methano-1H- | Dihydro Cyclacet | 0.01630000 |
| | | inden-5-ol, | | |
| | | octahydro-, 5-acetate | | |
| 142. | 95-41-0 | 2-Cyclopenten-1- | Iso Jasmone T | 0.01600000 |
| | | one, 2-hexyl- | | |
| 143. | 134-20-3 | Benzoic acid, 2- | Methyl | 0.01580000 |
| | | amino-, methyl ester | Anthranilate | |
| 144. | 100-06-1 | Ethanone, 1-(4- | Para Methoxy | 0.01550000 |
| | | methoxyphenyl)- | Acetophenone | |
| 145. | 105-86-2 | 2,6-Octadien-1-ol, | Geranyl Formate | 0.01540000 |
| | | 3,7-dimethyl-, 1- | | |
| | | formate, (2E)- | | |
| 146. | 154171-77-4 | Spiro[1,3-dioxolane- | Ysamber K ® | 0.01470000 |
| | | 2,8′(5′H)-[2H- | | |
| | | 2,4a]methanonaphthalene], | | |
| | | hexahydro- | | |
| | | 1′,1′,5′,5′- | | |
| | | tetramethyl- | | |
| | | (2′S,4′aS,8′aS)- | | |
| | | (9CI) | | |
| 147. | 154171-76-3 | Spiro[1,3-dioxolane- | Ysamber | 0.01470000 |
| | | 2,8′(5′H)-[2H- | | |
| | | 2,4a]methanonaphthalene], | | |
| 148. | 127-41-3 | 3-Buten-2-one, 4- | Alpha-Ionone | 0.01440000 |
| | | (2,6,6-trimethyl-2- | | |
| | | cyclohexen-1-yl)-, | | |
| | | (3E)- | | |
| 149. | 151-05-3 | Benzeneethanol, | Dimethyl Benzyl | 0.01390000 |
| | | α,α-dimethyl-, 1- | Carbinyl Acetate | |
| | | acetate | | |
| 150. | 2500-83-6 | 4,7-Methano-1H- | Flor Acetate | 0.01370000 |
| | | inden-5-ol, | | |
| | | 3a,4,5,6,7,7a- | | |
| | | hexahydro-, 5- | | |
| | | acetate | | |
| 151. | 150-84-5 | 6-Octen-1-ol, 3,7- | Citronellyl | 0.01370000 |
| | | dimethyl-, 1-acetate | acetate | |
| 152. | 30310-41-9 | 2H-Pyran, | Pelargene | 0.01350000 |
| | | tetrahydro-2-methyl- | | |
| | | 4-methylene-6- | | |
| | | phenyl- | | |
| 153. | 68845-00-1 | Bicyclo[3.3.1]nonane, | Boisiris | 0.01350000 |
| | | 2-ethoxy-2,6,6- | | |
| | | trimethyl-9- | | |
| | | methylene- | | |
| 154. | 106-24-1 | 2,6-Octadien-1-ol, | Geraniol | 0.01330000 |
| | | 3,7-dimethyl-, (2E)- | | |
| 155. | 106-25-2 | 2,6-Octadien-1-ol, | Nerol | 0.01330000 |
| | | 3,7-dimethyl-, (2Z)- | | |
| 156. | 75975-83-6 | Bicyclo[7.2.0]undec- | Vetyvenal | 0.01280000 |
| | | 4-ene, 4,11,11- | | |
| | | trimethyl-8- | | |
| | | methylene-, | | |
| | | (1R,4E,9S)- | | |
| 157. | 19870-74-7 | 1H-3a,7- | Cedryl methyl | 0.01280000 |
| | | Methanoazulene, | ether | |
| | | octahydro-6- | | |
| | | methoxy-3,6,8,8- | | |
| | | tetramethyl-, | | |
| | | (3R,3aS,6S,7R,8aS)- | | |
| 158. | 87-44-5 | Bicyclo[7.2.0]undec- | Caryophyllene | 0.01280000 |
| | | 4-ene, 4,11,11- | Extra | |
| | | trimethyl-8- | | |
| | | methylene-, | | |
| | | (1R,4E,9S)- | | |
| 159. | 54440-17-4 | 1H-Inden-1-one, | Safraleine | 0.01260000 |
| | | 2,3-dihydro-2,3,3- | | |
| | | trimethyl- | | |
| 160. | 110-98-5 | 2-Propanol, 1,1′- | Dipropylene | 0.01250000 |
| | | oxybis- | Glycol | |
| 161. | 41890-92-0 | 2-Octanol, 7- | Osyrol ® | 0.01250000 |
| | | methoxy-3,7- | | |
| | | dimethyl- | | |
| 162. | 71077-31-1 | 4,9-Decadienal, 4,8- | Floral Super | 0.01230000 |
| | | dimethyl- | | |
| 163. | 65-85-0 | Benzoic Acid | Benzoic Acid | 0.01220000 |
| 164. | 61444-38-0 | 3-Hexenoic acid, | cis-3-hexenyl-cis- | 0.01220000 |
| | | (3Z)-3-hexen-1-yl | 3-hexenoate | |
| | | ester, (3Z)- | | |
| 165. | 116044-44-1 | Bicyclo[2.2.1]hept- | Herbanate | 0.01210000 |
| | | 5-ene-2-carboxylic | | |
| | | acid, 3-(1- | | |
| 166. | 104-54-1 | 2-Propen-1-ol, 3- | Cinnamic | 0.01170000 |
| | | phenyl- | Alcohol | |
| 167. | 78-35-3 | Propanoic acid, 2- | Linalyl | 0.01170000 |
| | | methyl-, 1-ethenyl- | Isobutyrate | |
| | | 1,5-dimethyl-4- | | |
| | | hexen-1-yl ester | | |
| 168. | 23495-12-7 | Ethanol, 2-phenoxy-, | Phenoxy Ethyl | 0.01130000 |
| | | 1-propanoate | Propionate | |
| 169. | 103-26-4 | 2-Propenoic acid, 3- | Methyl | 0.01120000 |
| | | phenyl-, methyl ester | Cinnamate | |
| 170. | 67634-14-4 | Benzenepropanal, 2- | Florazon (ortho- | 0.01110000 |
| | | ethyl-α,α-dimethyl- | isomer) | |
| 171. | 5454-19-3 | Propanoic acid, | N-Decyl | 0.01100000 |
| | | decyl ester | Propionate | |
| 172. | 93-16-3 | Benzene, 1,2- | Methyl Iso | 0.01100000 |
| | | dimethoxy-4-(1- | Eugenol | |
| | | propen-1-yl)- | | |
| 173. | 81782-77-6 | 3-Decen-5-ol, 4- | 4-Methyl-3- | 0.01070000 |
| | | methyl- | decen-5-ol | |
| 174. | 67845-30-1 | Bicyclo[2.2.2]oct-5- | Maceal | 0.01060000 |
| | | ene-2- | | |
| | | carboxaldehyde, 6- | | |
| | | methyl-8-(1- | | |
| | | methylethyl)- | | |
| 175. | 97-53-0 | Phenol, 2-methoxy- | Eugenol | 0.01040000 |
| | | 4-(2-propen-1-yl)- | | |
| 176. | 120-57-0 | 1,3-Benzodioxole-5- | Heliotropin | 0.01040000 |
| | | carboxaldehyde | | |
| 177. | 93-04-9 | Naphthalene, 2- | Beta Naphthyl | 0.01040000 |
| | | methoxy- | Methyl Ether | |
| | | | Extra 99 | |
| 178. | 4826-62-4 | 2-Dodecenal | 2 Dodecene-1-al | 0.01020000 |
| 179. | 20407-84-5 | 2-Dodecenal, (2E)- | Aldehyde | 0.01020000 |
| | | | Mandarin | |
| 180. | 5462-06-6 | Benzenepropanal, 4- | Canthoxal | 0.01020000 |
| | | methoxy-α-methyl- | | |
| 181. | 94-60-0 | 1,4- | Dimethyl 1,4- | 0.01020000 |
| | | Cyclohexanedicarboxylic | cyclohexanedicarboxylate | |
| | | acid, 1,4- | | |
| | | dimethyl ester | | |
| 182. | 57378-68-4 | 2-Buten-1-one, 1- | delta-Damascone | 0.01020000 |
| | | (2,6,6-trimethyl-3- | | |
| | | cyclohexen-1-yl)- | | |
| 183. | 17283-81-7 | 2-Butanone, 4- | Dihydro Beta | 0.01020000 |
| | | (2,6,6-trimethyl-1- | Ionone | |
| | | cyclohexen-1-yl)- | | |
| 184. | 1885-38-7 | 2-Propenenitrile, 3- | Cinnamalva | 0.01010000 |
| | | phenyl-, (2E)- | | |
| 185. | 103-48-0 | Propanoic acid, 2- | Phenyl Ethyl Iso | 0.00994000 |
| | | methyl-, 2- | Butyrate | |
| | | phenylethyl ester | | |
| 186. | 488-10-8 | 2-Cyclopenten-1- | Cis Jasmone | 0.00982000 |
| | | one, 3-methyl-2- | | |
| | | (2Z)-2-penten-1-yl- | | |
| 187. | 7492-67-3 | Acetaldehyde, 2- | Citronellyloxyacetaldehyde | 0.00967000 |
| | | [(3,7-dimethyl-6- | | |
| | | octen-1-yl)oxy]- | | |
| 188. | 68683-20-5 | 1-Cyclohexene-1- | Iso Bergamate | 0.00965000 |
| | | ethanol, 4-(1- | | |
| | | methylethyl)-, 1- | | |
| | | formate | | |
| 189. | 3025-30-7 | 2,4-Decadienoic | Ethyl 2,4- | 0.00954000 |
| | | acid, ethyl ester, | Decadienoate | |
| | | (2E,4Z)- | | |
| 190. | 103-54-8 | 2-Propen-1-ol, 3- | Cinnamyl Acetate | 0.00940000 |
| | | phenyl-, 1-acetate | | |
| 191. | 18127-01-0 | Benzenepropanal, 4- | Bourgeonal | 0.00934000 |
| | | (1,1-dimethylethyl)- | | |
| 192. | 3738-00-9 | Naphtho[2,1-b]furan, | Ambrox ® or | 0.00934000 |
| | | dodecahydro- | Cetalox ® or | |
| | | 3a,6,6,9a- | Synambran | |
| | | tetramethyl- | | |
| 193. | 51519-65-4 | 1,4- | Tamisone | 0.00932000 |
| | | Methanonaphthalen- | | |
| | | 5(1H)-one, | | |
| | | 4,4a,6,7,8,8a- | | |
| | | hexahydro- | | |
| 194 | 148-05-1 | Dodecanoic acid, | Dodecalactone | 0.00931000 |
| | | 12-hydroxy-, λ- | | |
| | | lactone (6CI,7CI); | | |
| | | 1,12- | | |
| 195. | 6790-58-5 | (3aR,5aS,9aS,9bR)- | Ambronat ® or | 0.00930000 |
| | | 3a,6,6,9a- | Ambroxan ® | |
| | | tetramethyl- | | |
| | | 2,4,5,5a,7,8,9,9b- | | |
| | | octahydro-1H- | | |
| | | benzo[e][1]benzofuran | | |
| 196. | 86-26-0 | 1,1′-Biphenyl, 2- | Methyl Diphenyl | 0.00928000 |
| | | methoxy- | Ether | |
| 197. | 68738-94-3 | 2- | Cyclomyral ® | 0.00920000 |
| | | Naphthalenecarboxaldehyde, | | |
| | | octahydro- | | |
| | | 8,8-dimethyl | | |
| 198. | 2705-87-5 | Cyclohexanepropanoic | Allyl | 0.00925000 |
| | | acid, 2-propen-1- | Cyclohexane | |
| | | yl ester | Propionate | |
| 199. | 7011-83-8 | 2(3H)-Furanone, 5- | Lactojasmone ® | 0.00885000 |
| | | hexyldihydro-5- | | |
| | | methyl- | | |
| 200. | 61792-11-8 | 2,6- | Lemonile ® | 0.00884000 |
| | | Nonadienenitrile, | | |
| | | 3,7-dimethyl- | | |
| 201. | 692-86-4 | 10-Undecenoic acid, | Ethyl | 0.00882000 |
| | | ethyl ester | Undecylenate | |
| 202. | 103-95-7 | Benzenepropanal, α- | Cymal | 0.00881000 |
| | | methyl-4-(1- | | |
| | | methylethyl)- | | |
| 203. | 13019-22-2 | 9-Decen-1-ol | Rosalva | 0.00879000 |
| 204. | 94201-19-1 | 1- | Methyl Laitone | 0.00872000 |
| | | Oxaspiro[4.5]decan- | 10% TEC | |
| | | 2-one, 8-methyl- | | |
| 205. | 104-61-0 | 2(3H)-Furanone, | γ-Nonalactone | 0.00858000 |
| | | dihydro-5-pentyl- | | |
| 206. | 706-14-9 | 2(3H)-Furanone, 5- | γ-Decalactone | 0.00852000 |
| | | hexyldihydro- | | |
| 207. | 24720-09-0 | 2-Buten-1-one, 1- | α-Damascone | 0.00830000 |
| | | (2,6,6-trimethyl-2- | | |
| | | cyclohexen-1-yl)-, | | |
| | | (2E)- | | |
| 208. | 39872-57-6 | 2-Buten-1-one, 1- | Isodamascone | 0.00830000 |
| | | (2,4,4-trimethyl-2- | | |
| | | cyclohexen-1-yl)-, | | |
| | | (2E)- | | |
| 209. | 705-86-2 | 2H-Pyran-2-one, | Decalactone | 0.00825000 |
| | | tetrahydro-6-pentyl- | | |
| 210. | 67634-15-5 | Benzenepropanal, 4- | Floralozone | 0.00808000 |
| | | ethyl-α,α-dimethyl- | | |
| 211. | 40527-42-2 | 1,3-Benzodioxole, 5- | Heliotropin | 0.00796000 |
| | | (diethoxymethyl)- | Diethyl Acetal | |
| 212. | 56973-85-4 | 4-Penten-1-one, 1- | Neobutenone α | 0.00763000 |
| | | (5,5-dimethyl-1- | | |
| | | cyclohexen-1-yl)- | | |
| 213. | 128-51-8 | Bicyclo[3.1.1]hept- | Nopyl Acetate | 0.00751000 |
| | | 2-ene-2-ethanol, 6,6- | | |
| | | dimethyl-, 2-acetate | | |
| 214. | 103-36-6 | 2-Propenoic acid, 3- | Ethyl Cinnamate | 0.00729000 |
| | | phenyl-, ethyl ester | | |
| 215. | 5182-36-5 | 1,3-Dioxane, 2,4,6- | Floropal ® | 0.00709000 |
| | | trimethyl-4-phenyl- | | |
| 216. | 42604-12-6 | Cyclododecane, | Boisambrene | 0.00686000 |
| | | (methoxymethoxy)- | | |
| 217. | 33885-52-8 | Bicyclo[3.1.1]hept- | Pinyl Iso Butyrate | 0.00685000 |
| | | 2-ene-2-propanal, | Alpha | |
| | | α,α,6,6-tetramethyl- | | |
| 218. | 92015-65-1 | 2(3H)- | Natactone | 0.00680000 |
| | | Benzofuranone, | | |
| | | hexahydro-3,6- | | |
| | | dimethyl- | | |
| 219. | 63767-86-2 | Cyclohexanemethanol, | Mugetanol | 0.00678000 |
| | | α-methyl-4-(1- | | |
| | | methylethyl)- | | |
| 220. | 3288-99-1 | Benzeneacetonitrile, | Marenil CI | 0.00665000 |
| | | 4-(1,1- | | |
| | | dimethylethyl)- | | |
| 221. | 35044-68-9 | 2-Buten-1-one, 1- | beta-Damascone | 0.00655000 |
| | | (2,6,6-trimethyl-1- | | |
| | | cyclohexen-1-yl)- | | |
| 222. | 41724-19-0 | 1,4- | Plicatone | 0.00652000 |
| | | Methanonaphthalen- | | |
| | | 6(2H)-one, | | |
| | | octahydro-7-methyl | | |
| 223. | 75147-23-8 | Bicyclo[3.2.1]octan- | Buccoxime ® | 0.00647000 |
| | | 8-one, 1,5-dimethyl-, | | |
| | | oxime | | |
| 224. | 25634-93-9 | 2-Methyl-5- | Rosaphen ® | 0.00637000 |
| | | phenylpentan-1-ol | 600064 | |
| 225. | 55066-48-3 | 3-Methyl-5- | Phenyl Hexanol | 0.00637000 |
| | | phenylpentanol | | |
| 226. | 495-62-5 | Cyclohexene, 4-(1,5- | Bisabolene | 0.00630000 |
| | | dimethyl-4-hexen-1- | | |
| | | ylidene)-1-methyl- | | |
| 227. | 2785-87-7 | Phenol, 2-methoxy- | Dihydro Eugenol | 0.00624000 |
| | | 4-propyl- | | |
| 228. | 87-19-4 | Benzoic acid, 2- | Iso Butyl | 0.00613000 |
| | | hydroxy-, 2- | Salicylate | |
| | | methylpropyl ester | | |
| 229. | 4430-31-3 | 2H-1-Benzopyran-2- | Octahydro | 0.00586000 |
| | | one, octahydro- | Coumarin | |
| 230. | 38462-22-5 | Cyclohexanone, 2- | Ringonol 50 TEC | 0.00585000 |
| | | (1-mercapto-1- | | |
| | | methylethyl)-5- | | |
| | | methyl- | | |
| 231. | 77-83-8 | 2-Oxiranecarboxylic | Ethyl Methyl | 0.00571000 |
| | | acid, 3-methyl-3- | Phenyl Glycidate | |
| | | phenyl-, ethyl ester | | |
| 232. | 37677-14-8 | 3-Cyclohexene-1- | Iso Hexenyl | 0.00565000 |
| | | carboxaldehyde, 4- | Cyclohexenyl | |
| | | (4-methyl-3-penten- | Carboxaldehyde | |
| | | 1-yl)- | | |
| 233. | 103-60-6 | Propanoic acid, 2- | Phenoxy Ethyl | 0.00562000 |
| | | methyl-, 2- | iso-Butyrate | |
| | | phenoxyethyl ester | | |
| 234. | 18096-62-3 | Indeno[1,2-d]-1,3- | Indoflor ® | 0.00557000 |
| | | dioxin, 4,4a,5,9b- | | |
| | | tetrahydro- | | |
| 235. | 63500-71-0 | 2H-Pyran-4-ol, | Florosa Q/Florol | 0.00557000 |
| | | tetrahydro-4-methyl- | | |
| | | 2-(2-methylpropyl)- | | |
| 236. | 65405-84-7 | Cyclohexanebutanal, | Cetonal ® | 0.00533000 |
| | | α,2,6,6-tetramethyl- | | |
| 237. | 171102-41-3 | 4,7-Methano-1H- | Flor Acetate | 0.00530000 |
| | | inden-6-ol, | | |
| | | 3a,4,5,6,7,7a- | | |
| | | hexahydro-8,8- | | |
| | | dimethyl-, 6-acetate | | |
| 238. | 10339-55-6 | 1,6-Nonadien-3-ol, | Ethyl linalool | 0.00520000 |
| | | 3,7-dimethyl- | | |
| 239. | 23267-57-4 | 3-Buten-2-one, 4- | Ionone Epoxide | 0.00520000 |
| | | (2,2,6-trimethyl-7- | Beta | |
| | | oxabicyclo[4.1.0]hept- | | |
| | | 1-yl)- | | |
| 240. | 97-54-1 | Phenol, 2-methoxy- | Isoeugenol | 0.00519000 |
| | | 4-(1-propen-1-yl)- | | |
| 241. | 67663-01-8 | 2(3H)-Furanone, 5- | Peacholide | 0.00512000 |
| | | hexyldihydro-4- | | |
| | | methyl- | | |
| 242. | 33885-52-8 | Bicyclo[3.1.1 ]hept- | Pinyl Iso Butyrate | 0.00512000 |
| | | 2-ene-2-propanal, | Alpha | |
| | | α,α,6,6-tetramethyl- | | |
| 243. | 23696-85-7 | 2-Buten-1-one, 1- | Damascenone | 0.00503000 |
| | | (2,6,6-trimethyl-1,3- | | |
| | | cyclohexadien-1-yl)- | | |
| 244. | 80-71-7 | 2-Cyclopenten-1- | Maple Lactone | 0.00484000 |
| | | one, 2-hydroxy-3- | | |
| | | methyl- | | |
| 245. | 67662-96-8 | Propanoic acid, 2,2- | Pivarose Q | 0.00484000 |
| | | dimethyl-, 2- | | |
| | | phenylethyl ester | | |
| 246. | 2437-25-4 | Dodecanenitrile | Clonal | 0.00480000 |
| 247. | 141-14-0 | 6-Octen-1-ol, 3,7- | Citronellyl | 0.00469000 |
| | | dimethyl-, 1- | Propionate | |
| | | propanoate | | |
| 248. | 54992-90-4 | 3-Buten-2-one, 4- | Myrrhone | 0.00460000 |
| | | (2,2,3,6- | | |
| | | tetramethylcyclohexyl)- | | |
| 249. | 55066-49-4 | Benzenepentanal, β- | Mefranal | 0.00455000 |
| | | methyl- | | |
| 250. | 7493-74-5 | Acetic acid, 2- | Allyl Phenoxy | 0.00454000 |
| | | phenoxy-, 2-propen- | Acetate | |
| | | 1-yl ester | | |
| 251. | 80-54-6 | Benzenepropanal, 4- | Lilial ® | 0.00444000 |
| | | (1,1-dimethylethyl)- | | |
| | | α-methyl- | | |
| 252. | 86803-90-9 | 4,7-Methano-1H- | Scentenal ® | 0.00439000 |
| | | indene-2- | | |
| | | carboxaldehyde, | | |
| | | octahydro-5- | | |
| | | methoxy- | | |
| 253. | 68991-97-9 | 2- | Melafleur | 0.00436000 |
| | | Naphthalenecarboxaldehyde, | | |
| | | 1,2,3,4,5,6,7,8- | | |
| | | octahydro-8,8- | | |
| | | dimethyl- | | |
| 254. | 18871-14-2 | Pentitol, 1,5- | Jasmal | 0.00434000 |
| | | anhydro-2,4- | | |
| | | dideoxy-2-pentyl-, | | |
| | | 3-acetate | | |
| 255. | 58567-11-6 | Cyclododecane, | Boisambren Forte | 0.00433000 |
| | | (ethoxymethoxy)- | | |
| 256. | 94400-98-3 | Naphth[2,3- | Molaxone | 0.00425000 |
| | | b]oxirene, | | |
| | | 1a,2,3,4,5,6,7,7a- | | |
| | | octahydro- | | |
| | | 1a,3,3,4,6,6- | | |
| | | hexamethyl-, | | |
| | | (1aR,4S,7aS)-rel- | | |
| 257. | 79-69-6 | 3-Buten-2-one, 4- | alpha-Irone | 0.00419000 |
| | | (2,5,6,6-tetramethyl- | | |
| | | 2-cyclohexen-1-yl)- | | |
| 258. | 65442-31-1 | Quinoline, 6-(1- | Iso Butyl | 0.00408000 |
| | | methylpropyl)- | Quinoline | |
| 259. | 87731-18-8 | Carbonic acid, 4- | Violiff | 0.00401000 |
| | | cycloocten-1-yl | | |
| | | methyl ester | | |
| 260. | 173445-65-3 | 1H-Indene-5- | Hivernal (A- | 0.00392000 |
| | | propanal, 2,3- | isomer); Portugal | |
| | | dihydro-3,3- | | |
| | | dimethyl- | | |
| 261. | 23911-56-0 | Ethanone, 1-(3- | Nerolione | 0.00383000 |
| | | methyl-2- | | |
| | | benzofuranyl)- | | |
| 262. | 52474-60-9 | 3-Cyclohexene-1- | Precyclemone B | 0.00381000 |
| | | carboxaldehyde, 1- | | |
| | | methyl-3-(4-methyl- | | |
| | | 3-penten-1-yl)- | | |
| 263. | 139539-66-5 | 6-Oxabicyclo | Cassifix | 0.00381000 |
| | | [3.2.1]octane, 5- | | |
| | | methyl-1-(2,2,3- | | |
| | | trimethyl-3- | | |
| | | cyclopenten-1-yl)- | | |
| 264. | 80858-47-5 | Benzene, [2- | Phenafleur | 0.00380000 |
| | | (cyclohexyloxy)ethyl]- | | |
| 265. | 32764-98-0 | 2H-Pyran-2-one, | Jasmolactone | 0.00355000 |
| | | tetrahydro-6-(3- | | |
| | | penten-1-yl)- | | |
| 266. | 78417-28-4 | 2,4,7-Decatrienoic | Ethyl 2,4,7- | 0.00353000 |
| | | acid, ethyl ester | decatrienoate | |
| 267. | 140-26-1 | Butanoic acid, 3- | Beta Phenyl Ethyl | 0.00347000 |
| | | methyl-, 2- | Isovalerate | |
| | | phenylethyl ester | | |
| 268. | 105-90-8 | 2,6-Octadien-1-ol, | Geranyl | 0.003360000 |
| | | 3,7-dimethyl-, 1- | Propionate | |
| | | propanoate, (2E)- | | |
| 269. | 41816-03-9 | Spiro[1,4- | Rhubofix ® | 0.00332000 |
| | | methanonaphthalene- | | |
| | | 2(1H),2′-oxirane], | | |
| | | 3,4,4a,5,8,8a- | | |
| | | hexahydro-3′,7- | | |
| | | dimethyl- | | |
| 270. | 7070-15-7 | Ethanol, 2- | Arbanol | 0.00326000 |
| | | [[(1R,2R,4R)-1,7,7- | | |
| | | trimethylbicyclo[2.2.1]hept- | | |
| | | 2-yl]oxy]-, rel- | | |
| 271. | 93-29-8 | Phenol, 2-methoxy- | Iso Eugenol | 0.00324000 |
| | | 4-(1-propen-1-yl)-, | Acetate | |
| | | 1-acetate | | |
| 272. | 476332-65-7 | 2H-Indeno[4,5- | Amber Xtreme | 0.00323000 |
| | | b]furan, decahydro- | Compound 1 | |
| | | 2,2,6,6,7,8,8- | | |
| | | heptamethyl- | | |
| 273. | 68901-15-5 | Acetic acid, 2- | Cyclogalbanate | 0.00323000 |
| | | (cyclohexyloxy)-, 2- | | |
| | | propen-1-yl ester | | |
| 274. | 107-75-5 | Octanal, 7-hydroxy- | Hydroxycitronellal | 0.00318000 |
| | | 3,7-dimethyl- | | |
| 275. | 68611-23-4 | Naphtho[2,1- | Grisalva | 0.00305000 |
| | | b]furan, 9b- | | |
| | | ethyldodecahydro- | | |
| | | 3a,7,7-trimethyl- | | |
| 276. | 313973-37-4 | 1,6-Heptadien-3- | Pharaone | 0.00298000 |
| | | one, 2-cyclohexyl- | | |
| 277. | 137-00-8 | 5-Thiazoleethanol, | Sulfurol | 0.00297000 |
| | | 4-methyl- | | |
| 278. | 7779-30-8 | 1-Penten-3-one, 1- | Methyl Ionone | 0.00286000 |
| | | (2,6,6-trimethyl-2- | | |
| | | cyclohexen-1-yl)- | | |
| 279. | 127-51-5 | 3-Buten-2-one, 3- | Isoraldeine Pure | 0.00282000 |
| | | methyl-4-(2,6,6- | | |
| | | trimethyl-2- | | |
| | | cyclohexen-1-yl)- | | |
| 280. | 72903-27-6 | 1,4- | Fructalate ™ | 0.00274000 |
| | | Cyclohexanedicarboxylic | | |
| | | acid, 1,4- | | |
| | | diethyl ester | | |
| 281. | 7388-22-9 | 3-Buten-2-one, 4- | Ionone Gamma | 0.00272000 |
| | | (2,2-dimethyl-6- | Methyl | |
| | | methylenecyclohexyl)- | | |
| | | 3-methyl- | | |
| 282. | 104-67-6 | 2(3H)-Furanone, 5- | gamma- | 0.00271000 |
| | | heptyldihydro- | Undecalactone | |
| | | | (racemic) | |
| 283. | 1205-17-0 | 1,3-Benzodioxole-5- | Helional | 0.00270000 |
| | | propanal, α-methyl- | | |
| 284. | 33704-61-9 | 4H-Inden-4-one, | Cashmeran | 0.00269000 |
| | | 1,2,3,5,6,7- | | |
| | | hexahydro-1,1,2,3,3- | | |
| | | pentamethyl- | | |
| 285. | 36306-87-3 | Cyclohexanone, 4- | Kephalis | 0.00269000 |
| | | (1-ethoxyethenyl)- | | |
| | | 3,3,5,5-tetramethyl- | | |
| 286. | 97384-48-0 | Benzenepropanenitrile, | Citrowanil ® B | 0.00265000 |
| | | α-ethenyl-α- | | |
| | | methyl- | | |
| 287. | 141-13-9 | 9-Undecenal, 2,6,10- | Adoxal | 0.00257000 |
| | | trimethyl- | | |
| 288. | 2110-18-1 | Pyridine, 2-(3- | Corps Racine VS | 0.00257000 |
| | | phenylpropyl)- | | |
| 289. | 27606-09-3 | Indeno[1,2-d]-1,3- | Magnolan | 0.00251000 |
| | | dioxin, 4,4a,5,9b- | | |
| | | tetrahydro-2,4- | | |
| | | dimethyl- | | |
| | 57082-24-3 | Caryophyllene | Caryophyllene | 0.00025000 |
| | | acetate | acetate | |
| 290. | 67634-20-2 | Propanoic acid, 2- | Cyclabute | 0.00244000 |
| | | methyl-, | | |
| | | 3a,4,5,6,7,7a- | | |
| | | hexahydro-4,7- | | |
| | | methano-1H-inden- | | |
| | | 5-yl ester | | |
| 291. | 65405-72-3 | 1-Naphthalenol, | Oxyoctaline | 0.00236000 |
| | | 1,2,3,4,4a,7,8,8a- | Formate | |
| | | octahydro-2,4a,5,8a- | | |
| | | tetramethyl-, 1- | | |
| | | formate | | |
| 292. | 122-40-7 | Heptanal, 2- | Amyl Cinnamic | 0.00233000 |
| | | (phenylmethylene)- | Aldehyde | |
| 293. | 103694-68-4 | Benzenepropanol, | Majantol ® | 0.00224000 |
| | | β,β,3-trimethyl- | | |
| 294. | 13215-88-8 | 2-Cyclohexen-1-one, | Tabanone Coeur | 0.00223000 |
| | | 4-(2-buten-1- | | |
| | | ylidene)-3,5,5- | | |
| | | trimethyl- | | |
| 295. | 25152-85-6 | 3-Hexen-1-ol, 1- | Cis-3-Hexenyl | 0.00203000 |
| | | benzoate, (3Z)- | Benzoate | |
| 296. | 406488-30-0 | 2-Ethyl-N-methyl-N- | Paradisamide | 0.00200000 |
| | | (m-tolyl)butanamide | | |
| 297. | 121-33-5 | Benzaldehyde, 4- | Vanillin | 0.00194000 |
| | | hydroxy-3-methoxy- | | |
| 298. | 77-54-3 | 1H-3a,7- | Cedac | 0.00192000 |
| | | Methanoazulen-6-ol, | | |
| | | octahydro-3,6,8,8- | | |
| | | tetramethyl-, 6- | | |
| | | acetate, | | |
| | | (3R,3aS,6R,7R,8aS)- | | |
| 299. | 76842-49-4 | 4,7-Methano-1H- | Frutene | 0.00184000 |
| | | inden-6-ol, | | |
| | | 3a,4,5,6,7,7a- | | |
| | | hexahydro-8,8- | | |
| | | dimethyl-, 6- | | |
| | | propanoate | | |
| 300. | 121-39-1 | 2-Oxiranecarboxylic | Ethyl Phenyl | 0.00184000 |
| | | acid, 3-phenyl-, | Glycidate | |
| | | ethyl ester | | |
| 301. | 211299-54-6 | 4H-4a,9- | Ambrocenide ® | 0.00182000 |
| | | Methanoazuleno[5,6- | | |
| | | d]-1,3-dioxole, | | |
| | | octahydro- | | |
| | | 2,2,5,8,8,9a- | | |
| | | hexamethyl-, | | |
| | | (4aR,5R,7aS,9R)- | | |
| 302. | 285977-85-7 | (2,5-Dimethyl-1,3- | Lilyflore | 0.00180000 |
| | | dihydroinden-2- | | |
| | | yl)methanol | | |
| 303. | 10094-34-5 | Butanoic acid, 1,1- | Dimethyl Benzyl | 0.00168000 |
| | | dimethyl-2- | Carbinyl Butyrate | |
| | | phenylethyl ester | | |
| 304. | 40785-62-4 | Cyclododeca[c] | Muscogene, | 0.00163000 |
| | | furan, 1,3,3a,4,5,6,7, | Portugal | |
| | | 8,9,10,11,13a- | | |
| | | dodecahydro- | | |
| 305. | 75490-39-0 | Benzenebutanenitrile, | Khusinil, Portugal | 0.00162000 |
| | | α,α,γ-trimethyl- | | |
| 306. | 55418-52-5 | 2-Butanone, 4-(1,3- | Dulcinyl | 0.00161000 |
| | | benzodioxol-5-yl)- | | |
| 307. | 3943-74-6 | Benzoic acid, 4- | Carnaline | 0.00157000 |
| | | hydroxy-3-methoxy-, | | |
| | | methyl ester | | |
| 308. | 72089-08-8 | 3-Cyclopentene-1- | Brahmanol ® | 0.00154000 |
| | | butanol, β,2,2,3- | | |
| | | tetramethyl- 2- | | |
| | | Methyl-4-(2,2,3- | | |
| | | trimethyl-3- | | |
| | | cyclopenten-1- | | |
| | | yl)butanol | | |
| 309. | 3155-71-3 | 2-Butenal, 2-methyl- | Boronal | 0.00147000 |
| | | 4-(2,6,6-trimethyl-1- | | |
| | | cyclohexen-1-yl)- | | |
| 310. | 2050-08-0 | Benzoic acid, 2- | Amyl Salicylate | 0.00144000 |
| | | hydroxy-, pentyl | | |
| | | ester | | |
| 311. | 41199-20-6 | 2-Naphthalenol, | Ambrinol | 0.00140000 |
| | | decahydro-2,5,5- | | |
| | | trimethyl- | | |
| 312. | 12262-03-2 | ndecanoic acid, 3- | Iso Amyl | 0.00140000 |
| | | methylbutyl ester | Undecylenate | |
| 313. | 107-74-4 | 1,7-Octanediol, 3,7- | Hydroxyol | 0.00139000 |
| | | dimethyl- | | |
| 314. | 91-64-5 | 2H-1-Benzopyran-2- | Coumarin | 0.00130000 |
| | | one | | |
| 315. | 68901-32-6 | 1,3-Dioxolane, 2-[6- | Glycolierral | 0.00121000 |
| | | methyl-8-(1- | | |
| | | methylethyl)bicyclo[ | | |
| | | 2.2.2]oct-5-en-2-yl]- | | |
| 316. | 68039-44-1 | Propanoic acid, 2,2- | Pivacyclene | 0.00119000 |
| | | dimethyl-, 3a,4,5,6, | | |
| | | 7,7a-hexahydro-4,7- | | |
| | | methano-1H-inden- | | |
| | | 6-yl ester | | |
| 317. | 106-29-6 | Butanoic acid, (2E)- | Geranyl Butyrate | 0.00116000 |
| | | 3,7-dimethyl-2,6- | | |
| | | octadien-1-yl ester | | |
| 318. | 5471-51-2 | 2-Butanone, 4-(4- | Raspberry ketone | 0.00106000 |
| | | hydroxyphenyl)- | | |
| 319. | 109-42-2 | 10-Undecenoic acid, | Butyl | 0.00104000 |
| | | butyl ester | Undecylenate | |
| 320. | 2785-89-9 | 4-Ethyl-2- | 4-Ethylguaiacol | 0.02000000 |
| | | methoxyphenol | | |
| | 27538-10-9 | 2-ethyl-4-hydroxy- | Homofuronol | 0.01210000 |
| | | 5-methylfuran-3-one | | |

*Vapor Pressures are acquired as described in the Test Methods Section.
**Origin: Same as for Table 1 hereinabove.

TABLE 2B
Moderate Volatile Natural Oils.

| No. | Natural oil | Supplier |
|---|---|---|
| 1. | Bay Oil Terpeneless | IFF |
| 2. | Cade Oil | H. Reynaud & Fils |
| 3. | Cedar Atlas Oil | Robertet |
| 4. | Cinnamon Bark Oil | Robertet |
| 5. | Cinnamon Oleoresin | Citrus & Allied Essences |
| 6. | Clove Bud Oil | Robertet |
| 7. | Clove Leaf Oil Rectified | H. Reynaud & Fils |
| 8. | Clove Stem Oil | H. Reynaud & Fils |
| 9. | Davana Oil | Robertet |
| 10. | Geranium Bourbon | Robertet |
| 11. | Ginger Oil Fresh Madagascar | IFF |
| 12. | Hay Absolute MD 50 PCT | IFF |
| 13. | Juniperberry Oil T'less | Robertet |
| 14. | Papyrus Oil | Robertet |
| 15. | Rose Absolute Oil | Robertet |
| 16. | Tonka Bean Absolute | Robertet |
| 17. | Wormwood Oil | Robertet |

Suppliers:
Citrus & Allied Essences, New York, USA
H. Reynaud & Fils, Montbrun-les-Bains, France
IFF, Hazlet, New Jersey, USA
Robertet, Grasse, France

Moderate volatile fragrance materials can be selected from the group of Tables 2A or 2B. However, it is understood by one skilled in the art that other moderate volatile fragrance materials, not recited in Tables 2A or 2B, would also fall within the scope of the present invention, so long as they have a vapor pressure of 0.1 to 0.001 Torr at 25° C.

### High Volatile Fragrance Materials

The fragrance component includes at least one high volatile fragrance material having a vapor pressure greater than 0.1 Torr (0.0133 kPa) at 25° C. In some examples, the high volatile fragrance material can include at least 2 high volatile fragrance materials, 3 high volatile fragrance materials, or at least 5 high volatile fragrance materials, or at least 7 high volatile fragrance materials. If there are more than one high volatile fragrance materials, then the ranges provided hereinabove cover the total of all of the high volatile fragrance materials. Suitable examples of high volatile fragrances materials are provided in Tables 3A and 3B below.

Preferably, the high volatile fragrance material is selected from at least 1 material, or at least 2 materials, or at least 3 materials, or at least 5 materials, at least 7 materials, or at least 9 high volatile fragrance materials as disclosed in Table 3A. Natural fragrance materials or oils having an aggregate vapour pressure greater than 0.1 Torr (0.0133 kPa) at 25° C. are provided in Table 3B. Moderate Volatile Natural Oils.

TABLE 3A
High Volatile Fragrance Materials

| | | | | Vapor |
|---|---|---|---|---|
| | CAS | | | Pressure (Torr |
| No. | Number | IUPAC Name | Common Name** | at 25° C.)* |
| 1. | 107-31-3 | Formic acid, | Methyl Formate | 732.00000000 |
| | | methyl ester | | |
| 2. | 75-18-3 | Methane, 1,1′- | Dimethyl Sulfide | 647.00000000 |
| | | thiobis- | 1.0% In DEP | |
| 3. | 141-78-6 | Acetic acid ethyl | Ethyl Acetate | 112.00000000 |
| | | ester | | |
| 4. | 105-37-3 | Propanoic acid, | Ethyl Propionate | 44.50000000 |
| | | ethyl ester | | |
| 5. | 110-19-0 | Acetic acid, 2- | Isobutyl Acetate | 18.00000000 |
| | | methylpropyl | | |
| | | ester | | |
| 6. | 105-54-4 | Butanoic acid, | Ethyl Butyrate | 13.90000000 |
| | | ethyl ester | | |
| 7. | 14765-30-1 | 1-Butanol | Butyl Alcohol | 8.52000000 |
| 8. | 7452-79-1 | Butanoic acid, 2- | Ethyl-2-Methyl | 7.85000000 |
| | | methyl-, ethyl | Butyrate | |
| | | ester | | |
| 9. | 123-92-2 | 1-Butanol, 3- | Iso Amyl Acetate | 5.68000000 |
| | | methyl-, 1- | | |
| | | acetate | | |
| 10. | 66576-71-4 | Butanoic acid, 2- | Iso Propyl 2- | 5.10000000 |
| | | methyl-, 1- | Methylbutyrate | |
| | | methylethyl ester | | |
| 11. | 110-43-0 | 2-Heptanone | Methyl Amyl | 4.73000000 |
| | | | Ketone | |
| 12. | 6728-26-3 | 2-Hexenal, (2E)- | Trans-2 Hexenal | 4.62000000 |
| 13. | 123-51-3 | 1-Butanol, 3- | Isoamyl Alcohol | 4.16000000 |
| | | methyl- | | |
| 14. | 1191-16-8 | 2-Buten-1-ol, 3- | Prenyl acetate | 3.99000000 |
| | | methyl-, 1- | | |
| | | acetate | | |
| 15. | 57366-77-5 | 1,3-Dioxolane-2- | Methyl Dioxolan | 3.88000000 |
| | | methanamine, N- | | |
| | | methyl- | | |
| 16. | 7785-70-8 | Bicyclo[3.1.1]hept- | Alpha Pinene | 3.49000000 |
| | | 2-ene, 2,6,6- | | |
| | | trimethyl-, | | |
| | | (1R,5R)- | | |
| 17. | 79-92-5 | Bicyclo[2.2.1]heptane, | Camphene | 3.38000000 |
| | | 2,2-dimethy1-3- | | |
| | | methylene- | | |
| 18. | 94087-83-9 | 2-Butanethiol, 4- | 4-Methoxy-2- | 3.31000000 |
| | | methoxy-2-methyl- | Methyl-2- | |
| | | | Butanenthiol | |
| 19. | 39255-32-8 | Pentanoic acid, | Manzanate | 2.91000000 |
| | | 2-methyl-, ethyl | | |
| | | ester | | |
| 20. | 3387-41-5 | Bicyclo[3.1.0]hexane, | Sabinene | 2.63000000 |
| | | 4-methylene-1-(1- | | |
| | | methylethyl)- | | |
| 21. | 127-91-3 | Bicyclo[3.1.1]heptane, | Beta Pinene | 2.40000000 |
| | | 6,6-dimethyl-2- | | |
| | | methylene- | | |
| 22. | 105-68-0 | 1-Butanol, 3- | Amyl Propionate | 2.36000000 |
| | | methyl-, 1- | | |
| | | propanoate | | |
| 23. | 123-35-3 | 1,6-Octadiene, 7- | Myrcene | 2.29000000 |
| | | methyl-3- | | |
| | | methylene- | | |
| 24. | 124-13-0 | Octanal | Octyl Aldehyde | 2.07000000 |
| 25. | 7392-19-0 | 2H-Pyran, 2- | Limetol | 1.90000000 |
| | | ethenyltetrahydro- | | |
| | | 2,6,6-trimethyl- | | |
| 26 | 111-13-7 | 2-Octanone | Methyl Hexyl | 1.72000000 |
| | | | Ketone | |
| 27. | 123-66-0 | Hexanoic acid, | Ethyl Caproate | 1.66000000 |
| | | ethyl ester | | |
| 28. | 470-82-6 | 2-Oxabicyclo[2.2.2]octane, | Eucalyptol | 1.65000000 |
| | | 1,3,3-trimethyl- | | |
| 29. | 99-87-6 | Benzene, 1- | Para Cymene | 1.65000000 |
| | | methyl-4-(1- | | |
| | | methylethyl)- | | |
| 30. | 104-93-8 | Benzene, 1- | Para Cresyl | 1.65000000 |
| | | methoxy-4- | Methyl Ether | |
| | | methyl- | | |
| 31. | 13877-91-3 | 1,3,6-Octatriene, | Ocimene | 1.56000000 |
| | | 3,7-dimethyl- | | |
| 32. | 138-86-3 | Cyclohexene, 1- | dl-Limonene | 1.54000000 |
| | | methyl-4-(1- | | |
| | | methylethenyl)- | | |
| 33. | 5989-27-5 | Cyclohexene, 1- | d-limonene | 1.54000000 |
| | | methyl-4-(1- | | |
| | | methylethenyl)-, (4R)- | | |
| 34. | 106-68-3 | 3-Octanone | Ethyl Amyl Ketone | 1.50000000 |
| 35. | 110-41-8 | Undecanal, 2- | Methyl Nonyl | 1.43000000 |
| | | methyl- | Acetaldehyde | |
| 36. | 142-92-7 | Acetic acid, | Hexyl acetate | 1.39000000 |
| | | hexyl ester | | |
| 37. | 110-93-0 | 5-Hepten-2-one, | Methyl Heptenone | 1.28000000 |
| | | 6-methyl- | | |
| 38. | 81925-81-7 | 2-Hepten-4-one, | Filbertone 1% in | 1.25000000 |
| | | 5-methyl- | TEC | |
| 39. | 3681-71-8 | 3-Hexen-1-ol, 1- | cis-3-Hexenyl | 1.22000000 |
| | | acetate, (3Z)- | acetate | |
| 40. | 97-64-3 | Propanoic acid, | Ethyl Lactate | 1.16000000 |
| | | 2-hydroxy-, | | |
| | | ethyl ester | | |
| 41. | 586-62-9 | Cyclohexene, 1- | Terpineolene | 1.13000000 |
| | | methyl-4-(1- | | |
| | | methylethylidene)- | | |
| 42. | 51115-64-1 | Butanoic acid, 2- | Amyl butyrate | 1.09000000 |
| | | methylbutyl ester | | |
| 43. | 106-27-4 | Butanoic acid, 3- | Amyl Butyrate | 1.09000000 |
| | | methylbutyl ester | | |
| 44. | 99-85-4 | 1,4- | Gamma Terpinene | 1.08000000 |
| | | Cyclohexadiene, | | |
| | | 1-methyl-4-(1- | | |
| | | methylethyl)- | | |
| 45. | 18640-74-9 | Thiazole, 2-(2- | 2-Isobutylthiazole | 1.07000000 |
| | | methylpropyl)- | | |
| 46. | 928-96-1 | 3-Hexen-1-ol, | cis-3-Hexenol | 1.04000000 |
| | | (3Z)- | | |
| 47. | 100-52-7 | Benzaldehyde | Benzaldehyde | 0.97400000 |
| 48. | 141-97-9 | Butanoic acid, 3- | Ethyl Acetoacetate; | 0.89000000 |
| | | oxo-, ethyl ester | Portugal | |
| 49. | 928-95-0 | 2-Hexen-1-ol, | Trans-2-Hexenol | 0.87300000 |
| | | (2E)- | | |
| 50. | 928-94-9 | 2-Hexen-1-ol, | Beta Gamma | 0.87300000 |
| | | (2Z)- | Hexenol | |
| 51. | 24691-15-4 | Cyclohexane, 3- | Herbavert | 0.85200000 |
| | | ethoxy-1,1,5- | | |
| | | trimethyl-, cis- | | |
| | | (9CI) | | |
| 52. | 19872-52-7 | 2-Pentanone, 4- | 4-Methyl-4- | 0.84300000 |
| | | mercapto-4- | Mercaptopentan-2- | |
| | | methyl- | one 1 ppm TEC | |
| 53. | 3016-19-1 | 2,4,6-Octatriene, | Allo-Ocimene | 0.81600000 |
| | | 2,6-dimethyl-, | | |
| | | (4E,6E)- | | |
| 54. | 69103-20-4 | Oxirane, 2,2- | Myroxide | 0.80600000 |
| | | dimethyl-3-(3- | | |
| | | methyl-2,4- | | |
| | | pentadien-1-yl)- | | |
| 55. | 189440-77-5 | 4,7-Octadienoic | Anapear | 0.77700000 |
| | | acid, methyl | | |
| | | ester, (4E)- | | |
| 56. | 67633-96-9 | Carbonic acid, | Liffarome ™ | 0.72100000 |
| | | (3Z)-3-hexen-1- | | |
| | | yl methyl ester | | |
| 57. | 123-68-2 | Hexanoic acid, | Allyl Caproate | 0.67800000 |
| | | 2-propen-1-yl | | |
| | | ester | | |
| 58. | 106-72-9 | 5-Heptenal, 2,6- | Melonal | 0.62200000 |
| | | dimethyl- | | |
| 59. | 106-30-9 | Heptanoic acid, | Ethyl Oenanthate | 0.60200000 |
| | | ethyl ester | | |
| 60. | 68039-49-6 | 3-Cyclohexene- | Ligustral or Triplal | 0.57800000 |
| | | 1-carboxaldehyde, | | |
| | | 2,4-dimethyl- | | |
| 61. | 101-48-4 | Benzene, (2,2- | Phenyl | 0.55600000 |
| | | dimethoxyethyl)- | Acetaldehyde | |
| | | | Dimethyl Acetal | |
| 62. | 16409-43-1 | 2H-Pyran, | Rose Oxide | 0.55100000 |
| | | tetrahydro-4- | | |
| | | methyl-2-(2- | | |
| | | methyl-1- | | |
| | | propen-1-yl)- | | |
| 63. | 925-78-0 | 3-Nonanone | Ethyl Hexyl Ketone | 0.55100000 |
| 64. | 100-47-0 | Benzonitrile | Benzyl Nitrile | 0.52400000 |
| 65. | 589-98-0 | 3-Octanol | Octanol-3 | 0.51200000 |
| 66. | 58430-94-7 | 1-Hexanol, | Iso Nonyl Acetate | 0.47000000 |
| | | 3,5,5-trimethyl-, | | |
| | | 1-acetate | | |
| 67. | 10250-45-0 | 4-Heptanol, 2,6- | Alicate | 0.45400000 |
| | | dimethyl-, 4- | | |
| | | acetate | | |
| 68. | 105-79-3 | Hexanoic acid, | Iso Butyl Caproate | 0.41300000 |
| | | 2-methylpropyl | | |
| | | ester | | |
| 69. | 2349-07-7 | Propanoic acid, | Hexyl isobutyrate | 0.41300000 |
| | | 2-methyl-, hexyl | | |
| | | ester | | |
| 70. | 23250-42-2 | Cyclohexanecarb- | Cyprissate | 0.40500000 |
| | | oxylic acid, 1,4- | | |
| | | dimethyl-, | | |
| | | methyl ester, | | |
| | | trans- | | |
| 71. | 122-78-1 | Benzeneacetaldehyde | Phenyl | 0.36800000 |
| | | | acetaldehyde | |
| 72. | 5405-41-4 | Butanoic acid, 3- | Ethyl-3-Hydroxy | 0.36200000 |
| | | hydroxy-, ethyl | Butyrate | |
| | | ester | | |
| 73. | 105-53-3 | Propanedioic | Diethyl Malonate | 0.34400000 |
| | | acid, 1,3-diethyl | | |
| | | ester | | |
| 74. | 93-58-3 | Benzoic acid, | Methyl Benzoate | 0.34000000 |
| | | methyl ester | | |
| 75. | 16356-11-9 | 1,3,5-Undecatriene | Undecatriene | 0.33600000 |
| 76. | 65405-70-1 | 4-Decenal, (4E)- | Decenal (Trans-4) | 0.33100000 |
| 77. | 54546-26-8 | 1,3-Dioxane, 2- | Herboxane | 0.33000000 |
| | | butyl-4,4,6- | | |
| | | trimethyl- | | |
| 78. | 13254-34-7 | 2-Heptanol, 2,6- | Dimethyl-2 6- | 0.33000000 |
| | | dimethyl- | Heptan-2-ol | |
| 79. | 98-86-2 | Ethanone, 1- | Acetophenone | 0.29900000 |
| | | phenyl- | | |
| 80. | 93-53-8 | Benzeneacetaldehyde, | Hydratropic | 0.29400000 |
| | | α-methyl- | aldehyde | |
| 81. | 80118-06-5 | Propanoic acid, | Iso Pentyrate | 0.28500000 |
| | | 2-methyl-, 1,3- | | |
| | | dimethyl-3- | | |
| | | buten-1-yl ester | | |
| 82. | 557-48-2 | 2,6-Nonadienal, | E Z-2,6-Nonadien- | 0.28000000 |
| | | (2E,6Z)- | 1-al | |
| 83. | 24683-00-9 | Pyrazine, 2- | 2-Methoxy-3- | 0.27300000 |
| | | methoxy-3-(2- | Isobutyl Pyrazine | |
| | | methylpropyl)- | | |
| 84. | 104-57-4 | Formic acid, | Benzyl Formate | 0.27300000 |
| | | phenylmethyl | | |
| | | ester | | |
| 85. | 104-45-0 | Benzene, 1- | Dihydroanethole | 0.26600000 |
| | | methoxy-4- | | |
| | | propyl- | | |
| 86. | 491-07-6 | Cyclohexanone, | Iso Menthone | 0.25600000 |
| | | 5-methyl-2-(1- | | |
| | | methylethyl)-, | | |
| | | (2R,5R)-rel- | | |
| 87. | 89-80-5 | Cyclohexanone, | Menthone Racemic | 0.25600000 |
| | | 5-methyl-2-(1- | | |
| | | methylethyl)-, | | |
| | | (2R,5S)-rel- | | |
| 88. | 2463-53-8 | 2-Nonenal | 2 Nonen-1-al | 0.25600000 |
| 89. | 55739-89-4 | Cyclohexanone, | Thuyacetone | 0.25000000 |
| | | 2-ethyl-4,4- | | |
| | | dimethyl- | | |
| 90. | 150-78-7 | Benzene, 1,4- | Hydroquinone | 0.25000000 |
| | | dimethoxy- | Dimethyl Ether | |
| 91. | 64988-06-3 | Benzene, 1- | Rosacene | 0.24600000 |
| | | (ethoxymethyl)- | | |
| | | 2-methoxy- | | |
| 92. | 76-22-2 | Bicyclo[2.2.1]heptan- | Camphor gum | 0.22500000 |
| | | 2-one, | | |
| | | 1,7,7-trimethyl- | | |
| 93. | 67674-46-8 | 2-Hexene, 6,6- | Methyl | 0.21400000 |
| | | dimethoxy-2,5,5- | Pamplemousse | |
| | | trimethyl- | | |
| 94. | 112-31-2 | Decanal | Decyl Aldehyde | 0.20700000 |
| 95. | 16251-77-7 | Benzenepropanal, | Trifernal | 0.20600000 |
| | | β-methyl- | | |
| 96. | 93-92-5 | Benzenemethanol, | Methylphenylcarbinol | 0.20300000 |
| | | α-methyl-, 1- | Acetate | |
| | | acetate | | |
| 97. | 143-13-5 | Acetic acid, | Nonyl Acetate | 0.19700000 |
| | | nonyl ester | | |
| 98. | 122-00-9 | Ethanone, 1-(4- | Para Methyl | 0.18700000 |
| | | methylphenyl)- | Acetophenone | |
| 99. | 24237-00-1 | 2H-Pyran, 6- | Gyrane | 0.18600000 |
| | | butyl-3,6- | | |
| | | dihydro-2,4- | | |
| | | dimethyl- | | |
| 100. | 41519-23-7 | Propanoic acid, | Hexenyl | 0.18200000 |
| | | 2-methyl-, (3Z)- | Isobutyrate | |
| | | 3-hexen-1-yl | | |
| | | ester | | |
| 101. | 93-89-0 | Benzoic acid, | Ethyl Benzoate | 0.18000000 |
| | | ethyl ester | | |
| 102. | 20780-48-7 | 3-Octanol, 3,7- | Tetrahydro Linalyl | 0.18000000 |
| | | dimethyl-, 3- | Acetate | |
| | | acetate | | |
| 103. | 101-41-7 | Methyl 2- | Methylphenyl | 0.17600000 |
| | | phenylacetate | acetate | |
| 104. | 40853-55-2 | 1-Hexanol, 5- | Tetrahydro | 0.17300000 |
| | | methyl-2-(1- | Lavandulyl Acetate | |
| | | methylethyl)-, 1- | | |
| | | acetate | | |
| 105. | 933-48-2 | Cyclohexanol, | Trimethylcyclohexanol | 0.17300000 |
| | | 3,3,5-trimethyl-, | | |
| | | (1R,5R)-rel- | | |
| 106. | 35158-25-9 | 2-Hexenal, 5- | Lactone of Cis | 0.17200000 |
| | | methyl-2-(1- | Jasmone | |
| | | methylethyl)- | | |
| 107. | 18479-58-8 | 7-Octen-2-ol, | Dihydromyrcenol | 0.16600000 |
| | | 2,6-dimethyl- | | |
| 108. | 140-11-4 | Acetic acid, | Benzyl acetate | 0.16400000 |
| | | phenylmethyl | | |
| | | ester | | |
| 109. | 14765-30-1 | Cyclohexanone, | 2-sec-Butyl Cyclo | 0.16300000 |
| | | 2-(1- | Hexanone | |
| | | methylpropyl)- | | |
| 110. | 20125-84-2 | 3-Octen-1-ol, | Octenol | 0.16000000 |
| | | (3Z)- | | |
| 111. | 142-19-8 | Heptanoic acid, | Allyl Heptoate | 0.16000000 |
| | | 2-propen-1-yl | | |
| | | ester | | |
| 112. | 100-51-6 | Benzenemethanol | Benzyl Alcohol | 0.15800000 |
| 113. | 10032-15-2 | Butanoic acid, 2- | Hexyl-2-Methyl | 0.15800000 |
| | | methyl-, hexyl | Butyrate | |
| | | ester | | |
| 114. | 695-06-7 | 2(3H)-Furanone, | Gamma | 0.15200000 |
| | | 5-ethyldihydro- | Hexalactone | |
| 115. | 21722-83-8 | Cyclohexaneethanol, | Cyclohexyl Ethyl | 0.15200000 |
| | | 1-acetate | Acetate | |
| 116. | 111-79-5 | 2-Nonenoic acid, | Methyl-2- | 0.14600000 |
| | | methyl ester | Nonenoate | |
| 117. | 16491-36-4 | Butanoic acid, | Cis 3 Hexenyl | 0.13500000 |
| | | (3Z)-3-hexen-1- | Butyrate; USA | |
| | | yl ester | | |
| 118. | 111-12-6 | 2-Octynoic acid, | Methyl Heptine | 0.12500000 |
| | | methyl ester | Carbonate | |
| 119. | 59323-76-1 | 1,3-Oxathiane, | Oxane | 0.12300000 |
| | | 2-methyl-4- | | |
| | | propyl-, (2R,4S)- | | |
| | | rel- | | |
| 120. | 62439-41-2 | Heptanal, 6- | Methoxy Melonal | 0.11900000 |
| | | methoxy-2,6- | | |
| | | dimethyl- | | |
| 121. | 13851-11-1 | Bicyclo[2.2.1]heptan- | Fenchyl Acetate | 0.11700000 |
| | | 2-ol, 1,3,3- | | |
| | | trimethyl-, 2- | | |
| | | acetate | | |
| 122. | 115-95-7 | 1,6-Octadien-3- | Linalyl acetate | 0.11600000 |
| | | ol, 3,7-dimethyl-, | | |
| | | 3-acetate | | |
| 123. | 18479-57-7 | 2-Octanol, 2,6- | Tetra-Hydro | 0.11500000 |
| | | dimethyl- | Myrcenol | |
| 124. | 78-69-3 | 3,7- | Tetra-Hydro | 0.11500000 |
| | | dimethyloctan-3- | Linalool | |
| | | ol | | |
| 125. | 111-87-5 | 1-Octanol | Octyl Alcohol | 0.11400000 |
| 126. | 71159-90-5 | 3-Cyclohexene- | Grapefruit | 0.10500000 |
| | | 1-methanethiol, | mercaptan | |
| | | α,α,4-trimethyl- | | |
| 127. | 80-25-1 | Cyclohexanemethanol, | Menthanyl Acetate | 0.10300000 |
| | | α,α,4-trimethyl-, | | |
| | | 1-acetate | | |
| 128. | 88-41-5 | Cyclohexanol, 2- | Verdox ™ | 0.10300000 |
| | | (1,1- | | |
| | | dimethylethyl)-, | | |
| | | 1-acetate | | |
| 129. | 32210-23-4 | Cyclohexanol, 4- | Vertenex | 0.10300000 |
| | | (1,1-dimethylethyl)-, | | |
| | | 1-acetate | | |
| 130. | 112-44-7 | Undecanal | n-Undecanal | 0.10200000 |
| 131. | 124-19-6 | Nonanal | Nonanal Aldehyde | 0.53200000 |
| | | | C-9 | |
| 132. | 929253-05-4 | 6-methoxy-2,6- | 6-methoxy-2,6- | 0.04020000 |
| | | dimethyloctanal | dimethyl octanal | |
| 133. | 68039-47-4 | 2-propan-2- | Phenethyl Isopropyl | 0.24900000 |
| | | yloxyethylbenzene | Ether | |
| 134. | 6413-10-1 | ethyl 2-(2- | Apple Ketal | 0.21900000 |
| | | methyl-1,3- | | |
| | | dioxolan-2- | | |
| | | yl)acetate | | |
| 135. | 106-23-0 | 3,7-dimethyloct- | citronellal | 0.21500000 |
| | | 6-enal | | |
| 136. | 14667-55-1 | Trimethyl | Trimethyl Pyrazine- | 1.72400000 |
| | | Pyrazine-2,3,5 | 2,3,5 | |

*Vapor Pressures are acquired as described in the Test Methods Section.
**Origin: Same as for Table 1 hereinabove.

TABLE 3B
High Volatile Fragrance Materials

| No. | Natural oil | Supplier |
|---|---|---|
| 1. | Angelica Seeds Oil | Robertet |
| 2. | Basil Oil Grand Vert | IFF |
| 3. | Bergamot Oil Reggio Early New Crop | Capua |
| 4. | Black Pepper Oil | Robertet |
| 5. | Blackcurrant Buds Absolute | Robertet |
| 6. | Cardamom Guatamala Extract CO2 | IFF |
| 7. | Cardamom Oil Guatemala | IFF |
| 8. | Cedarleaf Oil | Kerry |
| 9. | citronella oil | H. Reynaud & Fils |
| 10. | Clary Sage Oil French | IFF |
| 11. | Coffee Extract CO2 | Firmenich |
| 12. | Cucumber Extract | Firmenich |
| 13. | Cumin Oil | Robertet |
| 14. | Cypress Oil | IFF |
| 15. | Elemi Coeur Oil | Robertet |
| 16. | Ginger oil India | IFF |
| 17. | Grapefruit Zest | Citrus & Allied Essences |
| 18. | It. Bergamot Oil | Capua |
| 19. | Labdanum Cistus Absolute | Biolandes |
| 20. | Lavandin Grosso Oil | H. Reynaud & Fils |
| 21. | Lemon Oil Winter | Capua |
| 22. | Green Mandarin Oil | Simone Gatto |
| 23. | Nutmeg Oil | Robertet |
| 24. | Oil Orange Sinensal | Citrus & Allied Essences |
| 25. | Olibanum Oil Pyrogenous | Firmenich |
| 26. | Pepper Black CO2 Oil | Firmenich |
| 27. | Petitgrain Mandarinier Oil | Misitano & Stracuzzi |
| 28. | Pink Pepper CO2 OIL | Firmenich |
| 29. | Rum CO2 Oil | Firmenich |
| 30. | Sichuan Pepper CO2 oil | Firmenich |
| 31. | Styrax Resoid | IFF |
| 32. | Tangerine Oil | Robertet |
| 33. | Thym Oil | IFF |
| 34. | Violet Leaves Absolute | Robertet |

Suppliers
Biolandes, Le Sen, France
Capua, Campo Calabro, Italy
Citrus & Allied Essences, New York, USA
Firmenich, Geneva, Switzerland
Global Essence Inc, United Kingdom
H. Reynaud & Fils, Montbrun-les-Bains, France
IFF, Hazlet, New Jersey, USA
Kerry, Co. Kerry, Ireland
Mane, Le Bar-sur-Loup, France
Misitano & Stracuzzi, Messina, Italy
Robertet, Grasse, France
Simone Gatto, San Pierre Niceto, Italy

Exemplary high volatile fragrance materials selected from the group of Tables 3A or 3B are preferred. However, it is understood by one skilled in the art that other high volatile fragrance materials, not recited in Tables 3A or 3B, would also fall within the scope of the present invention, so long as they have a vapor pressure of greater than 0.1 Torr (0.0133 kPa) at 25° C.

The individual fragrance materials can be present in various concentrations of the fragrance component. For example in a “diamond construction” the low volatile material can be present in a range of from about 0 wt % to about 30 wt % of the fragrance component, about 10 wt % to about 20 wt %, less than equal to or greater than about 0 wt %, 5, 10, 15, 20, 25, or 30 wt %; the moderate volatile component can be present in a range of from about 30 wt % to about 70 wt % of the fragrance component, about 40 wt % to about 60 wt %, less than, equal to, or greater than about 30 wt %, 35, 40, 45, 50, 55, 60, 65, or about 70 wt %; the low volatile fragrance component can be present in a 0 wt % to about 30 wt % of the fragrance component, about 10 wt % to about 20 wt %, less than equal to or greater than about 0 wt %, 5, 10, 15, 20, 25, or 30 wt %.

In a “bottom heavy construction”

### (iv) Fragrance Modulators

The composition further includes at least one modulator as described herein below. Suitable examples of the fragrance modulators include:
the compound according to Formula I:

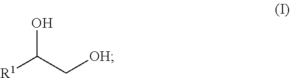

a polymer including a repeating unit derived from the compound according to Formula II:

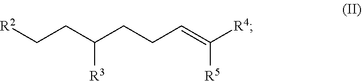

a mixture thereof. R¹, R², R³, R⁴, and R⁵, are independently chosen from —H, —OH, or substituted or unsubstituted (C₁-C₂₀)hydrocarbyl. In some further embodiments, R¹, R², R³, R⁴, and R⁵, are independently chosen from (C₁-C₂₀)alkyl, (C₂-C₂₀)alkenyl, (C₃-C₂₀)cycloalkyl, (C₁-C₂₀)alkoxyl, (C₁-C₂₀)aryl, or a combination thereof.

In some specific embodiments, the modulator component includes pentylene glycol, polycitronellol, or a mixture thereof. In some embodiments, the polycitronellol can include 2-8 repeating units and can have a weight average molecular weight in a range of from about 460 g/mol to about 1500 g/mol. In some embodiments, the modulator component can include a mixture of pentylene glycol and polycitronellol and a molar ratio of pentylene glycol to polycitronellol is in a range of from about 5:1 to about 1:5, about 4:1 to about 1:4, about 3:1 to about 1:3, or about 2:1 to about 1:2, or about 1:1. In some embodiments, the modulator component can include at least some PPG-20 methyl glucose ether mixed with any of the aforementioned modulators. Alternatively, the modulator component can be free of (include 0 wt %) PPG-20 methyl glucose. If PPG-20 methyl glucose is present, it can be in range of from about 1 wt % to about 15 wt % of the modulator component, about 5 wt % to about 10 wt % of the modulator component, less than, equal to, or greater than about 1 wt %, 2, 3, 4, 5, 6, 7, 8, 9, 10, 11, 12, 13, 14, or about 15 wt %.

In some further examples, the modulator component can include other modulators included in addition to pentylene glycol, polycitronellol, or a mixture thereof. Examples of the additional modulators can include those listed herein below in Tables 4(a) and 4(b).

Tables 4(a) and 4(b) provide lists of suitable non-odorous fragrance modulators.

TABLE 4(a)
Substantially Non-Odorous Fragrance Modulators

| | | | CAS | |
|---|---|---|---|---|
| No. | Group | Chemical Name | Number | Supplier |
| 1. | (a) | PPG-10 Methyl Glucose Ether | 61849-72-7 | Lubrizol |
| 2. | | PPG-20 Methyl Glucose Ether ¹ | 61849-72-7 | |
| 3. | | Ethoxylated Methyl Glucose Ether ² | 68239-42-9 | |
| 4. | | Caprylyl/Capryl Glucoside ³ | 68515-73-1 | BASF |
| 5. | | Undecyl Glucoside ^{3a} | — | SEPPIC |
| | | | | (France) |
| 6. | (b) | Isocetyl Alcohol ⁴ | 36653-82-4 | Ashland |
| | | | | Speciality |
| | | | | Ingredients |
| 7. | (c) | PPG-3 Myristyl Ether ⁵ | — | Evonik |
| 8. | | Neopentyl Glycol Diethylhexanoate ⁶ | 28510-23-8 | Lubrizol |
| 9. | (d) | Sucrose Laurate | 25339-99-5 | Alfa |
| | | | | Chemicals |
| | | | | Ltd. (UK) |
| 10. | | Sucrose dilaurate | 25915-57-5 | Alfa |
| | | | | Chemicals |
| | | | | Ltd. (UK) |
| 11. | | Sucrose Myristate | 27216-47-3 | Mitsubishi |
| | | | | Chemicals |
| 12. | | Sucrose Palmitate | 26446-38-8 | Alfa |
| | | | | Chemicals |
| 13. | | Sucrose Stearate | 25168-73-4 | Ltd. (UK) |
| 14. | | Sucrose Distearate | 27195-16-0 | Mitsubishi |
| | | | | Chemicals |
| | | | | (JP) |
| 15. | | Sucrose Tristearate | 27923063- | Mitsubishi |
| | | | 3 | Chemicals |
| | | | | (JP) |
| 16. | (e) | (E)-1-(2,2,6- | — | Takasago |
| | | trimethylcyclohexyl)oct-1-en-3- | | (Japan) |
| | | one ⁸ | | |
| 17. | (f) | 2-(1-menthoxy)ethane-1-ol ⁹ | — | Takasago |
| 18. | | 1-(1-menthoxy)propane-2-ol ⁹ | — | (Japan) |
| 19. | | 3-(1-menthoxy)propane-1-ol ⁹ | — | |
| 20. | | 3-(1-menthoxy)propane-1,2- | — | |
| | | diol ⁹ | | |
| 21. | | 2-methyl-3-(1- | — | |
| | | menthoxy)propane-1,2-diol ⁹ | | |
| 22. | | 4-(1-menthoxy) butane-1-ol ⁹ | — | |
| 23. | (g) | 1,1,4,4-tetramethyl-6-acetyl-7- | — | Givaudan |
| | | formyl-1,2,3,4- | | (Switzerland) |
| | | tetrahydronaphthalene ¹⁰ | | |
| 24. | | 1,1,2,4,4-pentamethyl-6-acetyl- | — | |
| | | 7-formyl-1,2,3,4- | | |
| | | tetrahydronaphthalene ¹⁰ | | |
| 25. | (h) | Hyaluronic acid disaccharide | 9004-61-9 | Sigma |
| | | sodium salt ¹¹ | | Aldrich |
| 26. | | Sodium Hyaluronate ¹¹ | 9067-32-7 | (UK) |
| 27. | (i) | Mono-o-(linalyl)-glucopyranose ¹² | — | Kanebo |
| 28. | | Di-o-(linalyl)-glucopyranose ¹² | — | (Japan) |
| 29. | | Tri-o-(linalyl)-glucopyranose ¹² | — | |
| 30. | | Tetra-o-(linalyl)-glucopyranose ¹² | — | |
| 31. | | Penta-o-(linaly1)-glucopyranose ¹² | — | |
| 32. | | Mono-o-(cis-3-hexenyl)- | — | |
| | | glactopyranose ¹² | | |
| 33. | | Di-o-(cis-3-hexenyl)- | — | |
| | | glactopyranose ¹² | | |
| 34. | | Tri-o-(cis-3-hexenyl)- | — | |
| | | glactopyranose ¹² | | |
| 35. | | Tetra-o-(cis-3-hexenyl)- | — | |
| | | glactopyranose ¹² | | |
| 36. | | Penta-o-(cis-3-hexenyl)- | — | |
| | | glactopyranose ¹² | | |
| 37. | (j) | Bis-O-(3,6-dioxadecanyl)- | — | |
| | | glucopyranose ¹³ | | |
| 38. | | Tris-O-(3,6-dioxadecanyl)- | — | |
| | | glucopyranose ¹³ | | |
| 39. | | Tetrakis-O-(3,6-dioxadecanyl)- | — | |
| | | glucopyranose ¹³ | | |
| 40. | | Pentakis-O-(3,6-dioxadecanyl)- | — | |
| | | glucopyranose ¹³ | | |
| 41. | | Bis-O-(3,6-dioxaoctanyl)- | — | |
| | | galactopyranose ¹³ | | |
| 42. | | Tris-O-(3,6-dioxaoctanyl)- | — | |
| | | galactopyranose ¹³ | | |
| 43. | | Tetrakis-O-(3,6-dioxaoctanyl)- | — | |
| | | galactopyranose ¹³ | | |
| 44. | | Pentakis-O-(3,6-dioxaoctanyl)- | — | |
| | | galactopyranose ¹³ | | |
| 45. | | Bis-O-(3,6-dioxaheptanyl)- | — | |
| | | xylopyranose ¹³ | | |
| 46. | | Tris-O-(3,6-dioxaheptanyl)- | — | |
| | | xylopyranose ¹³ | | |
| 47. | | Tetrakis-O-(3,6-dioxaheptanyl)- | — | |
| | | xylopyranose ¹³ | | |
| 48. | | Bis-O-(3,6-dioxadodecanyl)- | — | |
| | | glucopyranose ¹³ | | |
| 49. | | Tris-O-(3,6-dioxadodecanyl)- | — | |
| | | glucopyranose ¹³ | | |
| 50. | | Tetrakis-O-(3,6- | — | |
| | | dioxadodecanyl)-glucopyranose ¹³ | | |
| 51. | | Pentakis-O-(3,6-dioxadodecanyl)- | — | |
| | | glucopyranose ¹³ | | |
| 52. | (k) | Hydroquinone beta-D-glycoside ¹⁴ | 497-76-7 | Shiseido |
| 53. | (l) | Propylene Glycol Propyl Ether | 1569-01-3 | Sigma Aldrich |
| 54. | | Dicetyl Ether | 4113-12-6 | (UK) |
| 55. | | Polyglycerin-4 Ethers | 25618-55-7 | Solvay Chemicals |
| 56. | | Isoceteth-5 | 69364-63-2 | Nihon Emulsion |
| 57. | | Isoceteth-7 | 69364-63-2 | Company Ltd. |
| 58. | | Isoceteth-10 | 69364-63-2 | |
| 59. | | Isoceteth-12 | 69364-63-2 | |
| 60. | | Isoceteth-15 | 69364-63-2 | |
| 61. | | Isoceteth-20 | 69364-63-2 | |
| 62. | | Isoceteth-25 | 69364-63-2 | |
| 63. | | Isoceteth-30 | 69364-63-2 | |
| 64. | | Disodium | 68929-04-4 | Rhodia |
| | | Lauroamphodipropionate | | |
| 65. | | Hexaethylene glycol | 3055-96-7 | Sigma |
| | | monododecyl ether ^{14b} | | Aldrich |
| | | | | (UK) |
| 66. | (m) | Neopentyl Glycol | 27841-07-2 | Symrise |
| | | Diisononanoate ¹⁵ | | (Germany) |
| 67. | | Cetearyl Ethylhexnoate ¹⁶ | 90411-68-0 | |
| 68. | (n) | 2-ethylhexyloxypropanediol ¹⁷ | 70455-33-9 | Takasago |
| | | | | (JP) |
| 69. | (o) | Panthenol Ethyl Ether ¹⁸ | 667-83-4 | DSM |
| | | | | Nutritional |
| | | | | Products, |
| | | | | Inc. (USA) |
| 70. | | DL-Panthenol | 16485-10-2 | Roche Inc. |
| | | | | (USA) |
| 71. | (p) | Diisobutyl Adipate ¹⁹ | 141-04-8 | Sigma |
| | | | | Aldrich |
| 72. | | Diisoamyl Adipate ¹⁹ | 6624-70-0 | (UK) |
| 73. | (q) | PPG-11 Stearyl Ether ^{19a} | 25231-21-4 | Kao (JP) |
| 74. | (r) | N-hexadecyl n-nonanoate ^{19b} | 72934-15-7 | Symrise |
| | | (e.g., cetyl nonanoate) | | (Germany) |
| 75. | | Noctadecyl n-nonanoate ^{19b} | 107647-13- | |
| | | (e.g., stearyl nonanoate) | 2 | |
| 76. | (s) | methanone, (morphonyl) | — | Unilever |
| | | tricyclo[3.3.1.1 ^{3, 7}]dec-1-yl- ²⁰ | | (UK) |
| 77. | | methanone, (piperidinyl) | — | |
| | | tricyclo[3.3.1.1 ^{3, 7}]dec-1-yl- ²⁰ | | |
| 78. | | methanone, (pyrrolidinyl) | — | |
| | | tricyclo[3.3.1.1 ^{3, 7}]dec-1-yl ²⁰ | | |
| 79. | | methanone, (azetidinyl) | — | |
| | | tricyclo[3.3.1.1 ^{3, 7}]dec-1-yl- ²⁰ | | |
| 80. | | methanone, (hexahydroazepinyl) | — | |
| | | tricyclo[3.3.1.1 ^{3, 7}]dec-1-yl- ²⁰ | | |
| 81. | | methanone, (4-cyano- | — | |
| | | piperidinyl)tricyclo[3.3.1.1 ^{3, 7}]dec- | | |
| | | 1-yl- ²⁰ | | |
| 82. | | methanone, (4-amido- | (IT)— | |
| | | piperidinyl)tricyclo[3.3.1.1 ^{3, 7}]dec- | | |
| | | 1-yl- ²⁰ | | |
| 83. | | methanone, | — | |
| | | (Tricyclo[3.3.1.1 ^{3, 7}]decanyl)-N- | | |
| | | tricyclo[3.3.1.1 ^{3, 7}]dec-1-yl- ²⁰ | | |
| 84. | | methanone, | — | |
| | | (decahydroisoquinolinyl)tri- | | |
| | | cyclo[3.3.1.1 ^{3, 7}]dec-1-yl- ²⁰ | | |
| 85. | | methanone, | — | |
| | | (decahydroisoquinolinyl)tri- | | |
| | | cyclo[3.3.1.1 ^{3, 7}]dec-1-yl- ²⁰ | | |
| 86. | | methanone, | (IT)— | |
| | | (decahydroquinolinyl)tri- | | |
| | | cyclo[3.3.1.1 ^{3, 7}]dec-1-yl-20 | | |
| 87. | | methanone, (3,3-dimethyl-1- | — | |
| | | piperidinyl)tricyclo[3.3.1.1 ^{3, 7}] | | |
| | | dec-1-yl- ²⁰ | | |
| 88. | | methanone, (2-methyl-1- | — | |
| | | piperidinyl)tricyclo[3.3.1.1 ^{3, 7}] | | |
| | | dec-1-yl- 20 | | |
| 89. | | methanone, (4-methyl-1- | — | |
| | | piperidinyl)tricyclo[3.3.1.1 ^{3, 7}] | | |
| | | dec-1-yl- ²⁰ | | |
| 90. | | methanone, (3-methyl-1- | — | |
| | | piperidinyl)tricyclo[3.3.1.1 ^{3, 7}] | | |
| | | dec-1-yl- ²⁰ | | |
| 91. | | methanone, (3,5-dimethyl-1- | — | |
| | | piperidinyl)tricyclo[3.3.1.1 ^{3, 7}] | | |
| | | dec-1-yl- ²⁰ | | |
| 92. | | methanone, (4-methyl-4-ethy- | — | |
| | | piperidinyl)tricyclo[3.3.1.1 ^{3, 7}] | | |
| | | dec-1-yl- ²⁰ | | |
| 93. | | methanone, (3,3-diethyl-1- | — | |
| | | pyrrolidinyl)tricyclo[3.3.1.1 ^{3, 7}] | | |
| | | dec-1-yl- ²⁰ | | |
| 94. | | methanone, (N,N-diisopropyl) | — | |
| | | tricyclo[3.3.1.1 ^{3, 7}]dec-1-yl- ²⁰ | | |
| 95. | | methanone, (3,3- | — | |
| | | dimethylbutylaminyl) | | |
| | | tricyclo[3.3.1.1 ^{3, 7}]dec-1-yl- ²⁰ | | |
| 96. | | methanone, (2,2- | — | |
| | | dimethylpropylaminyl) | | |
| | | tricyclo[3.3.1.1 ^{3, 7}]dec-1-yl- ²⁰ | | |
| 97. | | methanone, (1,1-dimethyl-3,3- | — | |
| | | dimethylbutylaminyl) | | |
| | | tricyclo[3.3.1.1 ^{3, 7}]dec-1-yl- ²⁰ | | |
| 98. | | methanone, (1,3-dimethyl- | — | |
| | | butylaminyl) | | |
| | | tricycle[3.3.1.1 ^{3, 7}]dec-1-yl- ²⁰ | | |
| 99. | (t) | Bis-methoxy PEG-13 PEG- | 936645-35-1 | PolymerExpert |
| | | 438/PPG-110 SMDI Copolymer ²¹ | | S.A. (Pessac, |
| | | | | France) |
| 100. | (u) | propyl {4-[2-(diethylamino)-2- | 61791-12-6 | Sigma |
| | | oxoethoxy]-3- | | Aldrich |
| | | methoxyphenyl}acetate ²² | | (US) |
| 101. | (v) | 3-((2-ethylhexyl)oxy)propane- | 70445-33-9 | — |
| | | 1,2-diol ²³ | | |
| 102. | | 3-((2-propylheptyl)oxy)propane- | — | — |
| | | 1,2-diol ²³ | | |
| 103. | | 1-amino-3-((2- | 99509-00-9 | — |
| | | ethylhexyl)oxy)propan-2-01 ²³ | | |

¹ available as GLUCAM ™ P-20.
² available as Glucam ™ E-20.
³ available as Plantacare ® 810 UP.
^{3a} available as Simulsol ® SL 11W.
⁴ available as CERAPHYL ® ICA.
⁵ available as Tegosoft ® APM.
⁶ available as Schercemol ™ NGDO.
⁷ disclosed in U.S. Pat. No. 6,737,396B2 (Firmenich), column 1, lines 43-47.
⁸ diclosed as compound 1′i in U.S. Pat. No. 6,440,400B1 (Takasago Int. Corp.), col. 5.
^{8a} diclosed in U.S. Pat. No. 4,313,855 (Dragoco Gerberding & Co. GmbH), col. 1, lines 12-13.
⁹ disclosed in U.S. Pat. No. 7,538,081B2 (Takasago Int. Corp.), column 7, lines 50-53.
¹⁰ disclosed in U.S. Pat. No. 6,147,049 (Givaudan Roure), col. 5, line 24, to col. 6, line 17.
¹¹ disclosed in PCT Publication No. WO85/04803 (Diagnostic), pg. 2, line 1 to pg. 4, line 2.
¹² disclosed in JP Patent No. 61-083114 (Kanebo).
¹³ disclosed in JP Patent No. 61-063612 (Kanebo).
¹⁴ disclosed in JP Patent No. 62-084010 (Shiseido).
^{14b} available as: Laureth-6.
¹⁵ disclosed in U.S. Patent Publication No. 2011/0104089A1 (Symrise), para.
¹⁶ available as PCL-Liquid ® 100.
¹⁷ disclosed in U.S. Pat. No. 7,196,052 (Takasago Int. Corp.), col. 4, lines 34-35.
¹⁸ disclosed in EP Patent Publication No. 616800A2 (Givaudan), pg. 2, lines 12-25.
¹⁹ disclosed in U.S. Pat. No. 4,110,626 (Shiseido), column 3, lines 54-56.
^{19a} disclosed in PCT Publication No. WO2014/155019 (LVMH).
^{19b} disclosed in U.S. Pat. No. 9,050,261 (Symrise).
²⁰ disclosed as compounds C1-C22 in WO2014/139952 (Unilever).
²¹ available as Expert Gel ® EG56.
²² available as Kolliphor ® EL.
²³ disclosed in U.S. Pat. No. 9,050,261 (Symrise).

Further examples of non-odorous fragrance modulator is selected from the group of materials disclosed in Table 4(b).

TABLE 4(b)
Substantially Non-Ordorous Fragrance Modulators

| | Chemical or | | CAS | |
|---|---|---|---|---|
| No. | INCI Name | Trade Name | Number | Supplier |
| 1. | C12-14 Sec-Pareth-3 | Tergitol ® 15-S-7 | 68131-40-8 | Sigma Aldrich |
| | | | | (UK) |
| 2. | Poly(ethylene glycol- | PPG-7-Buteth-10 | 9038-95-3 | Sigma Aldrich |
| | ran-propylene glycol) | | | (UK) |
| | monobutyl ether | | | |
| 3. | PPG-4-Ceteth-10 | Nikkol PBC-33 | 37311-01-6 | Chemical Navi |
| 4. | Deceth-4 | Ethal DA-4 | 5703-94-6 | Ethox |
| | | | | Chemicals, Inc. |
| 5. | PPG-5-Ceteth-20 | AEC PPG-5-Ceteth-20 | 9087-53-0 | A & E Connock |
| | | | | (Perfumery & |
| | | | | Cosmetics) Ltd. |
| 6. | C14-15 Pareth-7 | Neodol 45-7 | 68951-67-7 | Shell Chemical |
| | | alcohol ethoxylate | | Company |
| 7. | Linear alcohol (C12-15) | Bio-soft N25-7 | 68131-39-5 | Stephan Company |
| | Pareth-3ethoxylate, POE-7 | | | (USA) |
| 8. | Linear alcohol (C12-13) | Bio-soft N23-6.5 | 66455-14-9 | |
| | Pareth-3ethoxylated, POE-6.5) | | | |
| 9. | Polyethylene glycol 1100 | Cremophor ® A 25 | 68439-49-6 | Sigma Aldrich |
| | mono(hexadecyl/octadecyl) | | | (UK) |
| | ether | | | |
| 10. | Linear alcohol (C9-11) | Bio-soft N91-8 | 68439-46-3 | Stephan Company |
| | ethoxylated POE-8 Pareth-3 | | | (USA) |
| 11. | Coceth-10 or Polyoxyethylene | Genapol ® C-100 | 61791-13-7 | Sigma Aldrich |
| | (10) dodecyl ether | | | (UK) |
| 12. | Alcohols, C12-14, ethoxylated | Rhodasurf ® LA 30 | 68439-50-9 | Solvay Solutions |
| | | | | Italia S.p.A. |
| 13. | Poly(ethylene glycol) | Poly(ethylene | 9004-74-4 | Sigma Aldrich |
| | methyl ether | glycol) methyl ether | | (UK) |
| 14. | C10-16 Pareth-1 | Neodol ® PC 110 | 68002-97-1 | Shell Chemical |
| | | | | Company |
| 15. | PPG-11 Stearyl Ether | Arlamol ™ PS11E | 25231-21-4 | Croda (UK) |
| 16. | Steareth-100 | Brij ® S100 | 9005-00-9 | Sigma Aldrich |
| | | | | (UK) |
| 17. | Polyethylene glycol | Brij ® C-58 | 9004-95-9 | Sigma Aldrich |
| | hexadecyl ether | | | (UK) |
| 18. | Pluronic ® F-127 | Pluronic ® F-127 | 9003-11-6 | Sigma Aldrich |
| | | | | (UK) |
| 19. | Linear Alcohol (C11) | Bio-soft N1-5 | 34398-01-1 | Stepan Canada |
| | Ethoxylate, POE-5 | | | Inc. |
| 20. | Laureth-10 | Intrasol FA 12/18/10 | 6540-99-4 | Evonik |
| | | | | Industries AG |
| 21. | Decaethylene glycol | Polyoxyethylene | 9002-92-0 | Sigma Aldrich |
| | mono-dodecyl ether | (10) lauryl ether | | (UK) |
| 22. | Ethylene glycol | 2-Methoxyethanol | 109-86-4 | Sigma Aldrich |
| | monomethyl ether | | | (UK) |
| 23. | Myreth-4 | Homulgator 920 G | 27306-79-2 | Grau Aromatics |
| | | | | GmbH & |
| | | | | Company KG |
| 24. | Oleth-16 Alkoxylated | Pegnol O-16A | 25190-05-0 | Toho Chemical |
| | Alcohols | | | Industry Co., Ltd. |
| 25. | Isosteareth-5 | Emalex 1805 | 52292-17-8 | Nihon Emulsion |
| | | | | Company, Ltd. |
| 26. | PPG-10 Cetyl Ether | Arlamol ™ PC10 | 9035-85-2 | Croda (UK) |
| 27. | Polyoxy(ethylene glycol) | Poly(ethylene glycol) | 24938-91-8 | Sigma-Aldrich |
| | (18) tridecyl ether | (18) tridecyl ether | | (UK) |
| 28. | Poly(oxy-1,2-ethanediyl), | ALFONIC ® 10-8 | 26183-52-8 | Sasol Chemicals |
| | a-decyl-w-hydroxy- | Ethoxylate | | (USA) LLC |
| 29. | Laureth- 1 | Mackam ™ 2LSF | 4536-30-5 | Rhodia (DE) |
| 30. | PEG-5 Hydrogenated | Ethox HTAM-5 | 61791-26-2 | Ethox |
| | Tallow Amine | | | Chemicals, Inc. |
| 31. | PEG-15 Oleamine | Nikkol TAMNO-15 | 26635-93-8 | Nikko Chemicals |
| | | | | Co., Ltd. |
| 32. | Polyoxyethylene (20) | Brij ® O20-SS | 9004-98-2 | Sigma Aldrich |
| | oleyl ether | | | (UK) |
| 33. | Cetoleth-10 | Brij ® CO10 | 8065-81-4 | Croda, Inc. |
| 34. | Talloweth-7 | Emulmin 70 | 61791-28-4 | Sanyo Chemical |
| | | | | Industries Ltd. |
| 35. | Isobutoxypropanol Alcohols | Isobutoxypropanol | 34150-35-1 | MolPort |
| 36. | Isobutoxypropanol Alcohols | Isobutoxypropanol | 23436-19-3 | AKos |
| | | | | Consulting & |
| | | | | Solutions |
| 37. | Diethylene Glycol | Twincide EDG | 111-46-6 | Roda |
| 38. | Methoxyethanol | Hisolve MC | 109-86-4 | Toho Chemical |
| | | | | Industry Co., Ltd. |
| 39. | Ethoxyethanol Alcohols | 2-Ethoxyethanol | 110-80-5 | Sigma-Aldrich |
| | | | | (UK) |
| 40. | Methoxyisopropanol Alcohols | Dowanol ™ PM | 107-98-2 | The Dow |
| | | | | Chemical Company |
| 41. | Methoxyethanol | Hisolve MC | 32718-54-0 | Toho Chemical |
| | | | | Industry Co., Ltd. |
| 42. | Methylal Ethers | Dimethoxymethane | 109-87-5 | Sigma-Aldrich |
| | | | | (UK) |
| 43. | 3-Methoxybutanol | Methoxybutanol | 2517-43-3 | Hans Schwarzkopf |
| | | | | GmbH/Co. KG |
| 44. | Butoxyethanol | Butyl OXITOL | 111-76-2 | Shell Chemical |
| | | | | Company |
| 45. | Propylene Glycol | Dowanol ™ PnB | 5131-66-8/ | The Dow |
| | n-Butyl Ether | | 29387-86-8 | Chemical Company |
| 46. | Propylene Glycol Butyl Ether | Propylene Glycol | 15821-83-7 | Sigma Aldrich |
| | | Butyl Ether | | (UK) |
| 47. | 2-(2-butoxyethoxy)ethanol | Diethylene glycol | 112-34-5 | Sigma Aldrich |
| | | butyl ether | | (UK) |
| 48. | Deceth-4 Phosphate | Crodafos ™ D4A | 52019-36-0 | Croda, Inc. |
| 49. | 2-(Hexadecyloxy)ethanol | Ethylene glycol | 2136-71-2 | Sigma-Aldrich |
| | | monohexadecyl ether | | (UK) |
| 50. | Poly(propylene glycol) | Poly(propylene glycol) | 9003-13-8 | Sigma-Aldrich |
| | monobutyl ether | monobutyl ether | | (UK) |
| 51. | Propylene Glycol | Dowanol ™ PnP | 30136-13-1 | The Dow |
| | Propyl Ether | | | Chemical Company |
| 52. | Propylene Glycol | Dowanol ™ PnB | 29387-86-8/ | The Dow |
| | n-Butyl Ether | | 5131-66-8 | Chemical Company |
| 53. | Dipropylene glycol | Di(propylene glycol) | 34590-94-8 | Sigma Aldrich |
| | monomethyl ether | methyl ether, mixture | | (UK) |
| | | of isomers | | |
| 54. | Dipropylene Glycol | Proglyde ™ DMM | 111109-77-4 | The Dow |
| | Dimethyl Ether | | | Chemical Company |
| 55. | PPG-2 Methyl Ether | Dowanol ™ DPM | 13429-07-7 | The Dow |
| | | | | Chemical Company |
| 56. | Methoxydiglycol Ethers | OriStar DEGME | 111-77-3 | Orient Stars LLC |
| 57. | Diethylene glycol | Di(ethylene glycol) | 111-90-0 | Sigma Aldrich |
| | ethyl ether | ethyl ether | | (UK) |
| 58. | Dimethoxydiglycol Ethers | Dimethyldiglycol | 111-96-6 | H&V Chemicals |
| 59. | PPG-3 Methyl Ether | Dowanol ™ TPM | 37286-64-9 | The Dow |
| | | | | Chemical Company |
| 60. | Methyl Morpholine | 224286 ALDRICH 4- | 7529-22-8 | Sigma-Aldrich |
| | Oxide Amine Oxides | Methylmorpholine N-oxide | | (UK) |
| 61. | Oleth-3 | Brij ® O3 | 5274-66-8 | Croda Europe, Ltd. |
| 62. | Tri(propylene glycol) | Dowanol ™ TPnB | 55934-93-5 | Sigma-Aldrich |
| | n-butyl ether | | | (UK) |
| 63. | Tripropylene Glycol | Tripropylene Glycol | 24800-44-0 | Sigma-Aldrich |
| | | | | (UK) |
| 64. | PPG-3 Methyl Ether | Dowanol ™ TPM | 25498-49-1 | The Dow |
| | Alkoxylated Alcohols | | | Chemical Company |
| 65. | Triethylene glycol | Triglycol | 112-27-6 | Sigma Aldrich |
| | | | | (UK) |
| 66. | PEG-3 Methyl Ether | Hymol ™ | 112-35-6 | Toho Chemical |
| | | | | Industry Co., Ltd |
| 67. | Laureth-3 | AEC Laureth-3 | 3055-94-5 | A & E Connock |
| | | | | (Perfumery & |
| | | | | Cosmetics) Ltd. |
| 68. | Ethylhexylglycerin | AG-G-75008 | 70445-33-9 | Angene Chemical |
| 69. | Tetra(ethylene glycol) | Tetraethylene glycol | 112-60-7 | Sigma Aldrich |
| | | | | (UK) |
| 70. | Steareth-3 | Isoxal 5 | 4439-32-1 | Vevy Europe SpA |
| 71. | Ceteth-3 | Emalex 103 | 4484-59-7 | Nihon Emulsion |
| | | | | Company, Ltd. |
| 72. | Myreth-3 | Isoxal 5 | 26826-30-2 | Vevy Europe SpA |
| 73. | Trideceth-3 | Alfonic ® TDA-3 | — | Sasol North |
| | | Ethoxylate | | America, Inc. |
| 74. | Ceteth-2 | Brij ® C2 | 5274-61-3 | Croda Europe, Ltd. |
| 75. | Oleth-2 | Brij ® O2 | 5274-65-7 | Croda, Inc. |
| 76. | Steareth-2 | Brij ® S2 | 16057-43-5 | Croda, Inc. |
| 77. | Cetoleth-10 | Brij ® CO10 | 8065-81-4 | Croda, Inc. |
| 78. | Trimethyl Pentanol | Trimethyl Pentanol | 68959-25-1 | Angene Chemical |
| | Hydroxyethyl Ether Alcohols | Hydroxyethyl Ether | | |
| 79. | Steareth-10 Allyl Ether | Salcare ® SC80 | 109292-17-3 | BASF |
| 80. | TEA-Lauryl Ether | material ID-AG-J-99109 | 1733-93-3 | Angene Chemical |
| 81. | Polyglyceryl-2 Oleyl Ether | Chimexane NB | 71032-90-1 | Chimex |
| 82. | Batyl Alcohol | B402 ALDRICH | 544-62-7 | Sigma-Aldrich |
| | | | | (UK) |
| 83. | Octaethylene Glycol | 15879 ALDRICH | 5117-19-1 | Sigma-Aldrich |
| | | | | (UK) |
| 84. | Triglycerol diisostearate | Cithrol ™ | 66082-42-6 | Croda (UK) |
| 85. | Diglycerin | Diglycerin 801 | 59113-36-9 | Sakamoto Yakuhin |
| | | | | Kogyo Co., Ltd. |
| 86. | Polyglycerin #310 | Polyglycerin #310 | 25618-55-7 | Sakamoto Yakuhin |
| | | | | Kogyo Co., Ltd. |
| 87. | Distearyl Ether | Cosmacol ® SE | 6297-03-6 | Sasol Germany |
| | | | | GmbH |
| 88. | Caprylyl Glyceryl Ether | Caprylyl Glyceryl Ether | 10438-94-5 | AKos |
| | | | | Consulting & |
| | | | | Solutions |
| 89. | Chimyl Alcohol | Chimyl Alcohol | 506-03-6 | Nikko Chemicals |
| | | | | Co., Ltd. |
| 90. | Dipentaerythrityl | Liponate ® DPC-6 | 68130-24-5 | Lipo Chemicals, |
| | Hexacaprylate/Hexacaprate | | | Inc. |
| 91. | Morpholine | 394467 ALDRICH | 110-91-8 | Sigma-Aldrich |
| | | | | (UK) |
| 92. | Dimethyl Oxazolidine | OXABAN ™-A | 51200-87-4 | The Dow |
| | | | | Chemical Company |
| 93. | Ethyl Hydroxymethyl | 4-Oxazolemethanol | 68140-98-7 | Angene Chemical |
| | Oleyl Oxazoline | | | |
| 94. | Methyl Hydroxymethyl | Adeka Nol GE-RF | 14408-42-5 | Adeka |
| | Oleyl Oxazoline | | | Corporation |
| 95. | Pramoxine HCl | OriStar PMHCL | 637-58-1 | Orient Stars |
| | | | | LLC |
| 96. | Allantoin Ascorbate | Allantoin Ascorbate | 57448-83-6 | ABI Chem |
| 97. | Stearamidopropyl | Mackalene ™ 326 | 55852-14-7 | Rhodia Inc. |
| | Morpholine Lactate | | | |
| 98. | Dioxolane | Elcotal DX | 646-06-0 | Lambiotte & |
| | | | | CIE S.A. |
| 99. | Glycerol Formal | Glycerol Formal | 5464-28-8 | Sigma Aldrich |
| | | | | (UK) |
| 100. | Stearamidopropyl | Mackine 321 | 55852-13-6 | Rhodia Inc. |
| | Morpholine | | | |
| 101. | 2,4,6- | Poly(melamine- | 68002-20-0 | Sigma-Aldrich |
| | Tris[bis(methoxymethyl)amino]- | co-formaldehyde) | | (UK) |
| | 1,3,5-triazine | methylated | | |
| 102. | Poloxamine 1307 | Pluracare ® 1307 | 11111-34-5 | BASF |
| 103. | Nonoxynol-8 | Igepal ® CO-610 | 27177-05-5 | Rhodia Inc. |
| 104. | Nonoxynol-10 | Igepal ® CO-710 | 27177-08-8 | Rhodia Inc. |
| 105. | Octoxynol-10 | Nikkol OP-10 | 2315-66-4 | Nikko Chemicals |
| | | | | Co., Ltd |
| 106. | Nonoxynol-9 | Igepal ® CO-630 | 68987-90-6 | Rhodia Inc. |
| 107. | Nonoxynol-9 Iodine | Nonoxynol-9 iodine | 94349-40-3 | Angene Chemical |
| 108. | Octylphenoxy | Igepal ® CA-630 | 68987-90-6 | Rhodia Inc. |
| | poly(ethyleneoxy)ethanol, | | | |
| | branched | | | |
| 109. | Sodium Octoxynol-2 | Triton ™ X-200 | 55837-16-6 | The Dow |
| | Ethane Sulfonate | | | Chemical Company |
| 110. | Benzylhemiformal | Preventol D2 | 14548-60-8 | Lanxess |
| | | | | Corporation |
| 111. | Nonoxynol-2 | Igepal ® CO-210 | 27176-93-8 | Rhodia Inc. |
| 112. | Octoxynol-3 | Igepal ® CA-420 | 2315-62-0 | The Dow |
| | | | | Chemical Company |
| 113. | Nonoxynol-3 | Marlophen NP 3 | 27176-95-0 | Sasol Germany |
| | | | | GmbH |
| 114. | Alkoxylated Alcohols | Alkasurf NP-4 | 7311-27-5 | Rhodia Inc. |
| 115. | Nonoxynol-3 | Triethylene Glycol | 51437-95-7 | Santa Cruz |
| | | Mono(p-nonylphenyl) | | Biotechnology |
| | | Ether | | |
| 116. | Nonoxynol-7 | Lowenol 2689 | 27177-03-3 | Jos. H. |
| | | | | Lowenstein & |
| | | | | Sons, Inc. (FR)_{—} |
| 117. | Nonoxynol-6 | Igepal ® CO-530 | 27177-01-1 | Rhodia Inc. |
| | | | | (FR) |
| 118. | Nonoxynol-5 | Igepal ® CO-520 | 20636-48-0 | Rhodia Inc. |
| 119. | Nonoxynol-5 | Igepal ® CO-520 | 26264-02-8 | Rhodia Inc. |
| 120. | Nonoxynol-4 | Alkasurf NP-4 | 27176-97-2 | Rhodia Inc. |
| 121. | Polyglyceryl-10 Trioleate | Nikkol Decaglyn 3-OV | 102051-00-3 | Nikko Chemicals |
| | | | | Co., Ltd. |
| 122. | Polyglyceryl-10 Dioleate | Nikkol Decaglyn 2-O | 33940-99-7 | Nikko Chemicals |
| | | | | Co., Ltd. |
| 123. | Polyglyceryl-10 Tetraoleate | Caprol 10G40 | 34424-98-1 | Abitec |
| | | | | Corporation |
| 124. | Polyglyceryl-10 Stearate | Nikkol Decaglyn | 79777-30-3 | Nikko Chemicals |
| | | 1-SV EX | | Co., Ltd. |
| 125. | Polyglyceryl-10 Oleate | S-Face O-1001 P | 79665-93-3 | Sakamoto Yakuhin |
| | | | | Kogyo Co., Ltd. |
| 126. | Polyglyceryl-10 Myristate | Nikkol Decaglyn | 87390-32-7 | Nikko Chemicals |
| | | 1-MV EX | | Co., Ltd. |
| 127. | Dermofeel ® G 10 L | Dermofeel ® G 10 L | 34406-66-1 | Dr. Straetmans |
| 128. | Polyglyceryl-6 Laurate | NIKKOL Hexaglyn 1-L | 51033-38-6 | Chemical Navi |
| 129. | Polyglyceryl-6 Isostearate | S-Face IS-601 P | 126928-07-2 | Sakamoto Yakuhin |
| | | | | Kogyo Co., Ltd. |
| 130. | Choleth-10 | Emalex CS-10 | 27321-96-6 | Nihon Emulsion |
| | | | | Company, Ltd. |
| 131. | Steareth-10 Allyl | Salcare ® SC80 | 109292-17-3 | BASF |
| | Ether/Acrylates Copolymer | | | |
| 132. | Polyvinyl Stearyl Ether | Giovarez ®1800 | 9003-96-7 | Phoenix |
| | | | | Chemical, Inc. |
| 133. | Dicetyl Ether | Cosmacol Ether 16 | — | Sasol Germany |
| | | | | GmbH |
| 134. | PPG-23-Steareth-34 | Unisafe 34S-23 | 9038-43-1 | Pola Chemical |
| | | | | Industries, Inc. |
| 135. | Stearoxypropyl Dimethylamine | Farmin DM E-80 | 17517-01-0 | Kao Corp. |
| 136. | Distearyl Ether | Cosmacol SE | 6297-03-6 | Sasol Germany |
| | | | | GmbH |
| 137. | Polyquaternium-10 | AEC Polyquaternium-10 | 55353-19-0 | A & E Connock |
| | | | | (Perfumery & |
| | | | | Cosmetics) Ltd. |
| 138. | Octyl ether | Dioctyl ether | 629-82-3 | Sigma Adlrich |
| | | | | (UK) |
| 139. | Ethyl Ether | Diethyl Ether | 60-29-7 | EMD Chemicals |
| 140. | Methyl Hexyl Ether Ethers | methyl hexyl ether | 4747-07-3 | TCI AMERICA |
| 141. | Ceteth-12 | Emalex 112 | 94159-75-8 | Nihon Emulsion |
| | | | | Company, Ltd. |
| 142. | Ceteth-10 or cetyl | Jeecol CA-10 | 14529-40-9 | Jeen |
| | alcohol POE-10 | | | International |
| 143. | Steareth-10 | Jeecol SA-10 | 13149-86-5 | Jeen |
| | | | | International |
| 144. | Nonaethylene glycol | Nonaethylene glycol | 3055-99-0 | Sigma Aldrich |
| | monododecyl ether | monododecyl ether | | (UK) |
| 145. | Oleth-10 | Brij ® O10 | 71976-00-6 | Croda, Inc. |
| 146. | Oleth-10 | Brij ® O10 | 24871-34-9 | Croda, Inc. |
| 147. | PEG-12 | Carbowax ™ PEG 600 | 6790-09-6 | The Dow |
| | | | | Chemical Company |
| 148. | PEG-9 | Sabopeg 400 | 3386-18-3 | Sabo s.p.a. |
| 149. | PEG-10 | DECAETHYLENE | 5579-66-8 | MolPort |
| | | GLYCOL | | |
| 150. | PEG-6 | Carbowax ™ PEG 300 | 2615-15-8 | The Dow |
| | | | | Chemical Company |
| 151. | Glycerol propoxylate | Glycerol propoxylate | 25791-96-2 | Sigma Aldrich |
| | | | | (UK) |
| 152. | Glycerol ethoxylate | Glycerol ethoxylate | 31694-55-0 | Sigma Aldrich |
| | | | | (UK) |
| 153. | Laureth-8 | AEC Laureth-8 | 3055-98-9 | A & E Connock |
| | | | | (Perfumery & |
| | | | | Cosmetics) Ltd. |
| 154. | Oleth-8 | Emalex 508 | 27040-03-5 | Nihon Emulsion |
| | | | | Company, Ltd. |
| 155. | Laureth-7 | Alfonic 1216CO-7 | 3055-97-8 | Sasol North |
| | | Ethoxylate | | America, Inc. |
| 156. | Steareth-7 | Polyoxyethylene (7) | 66146-84-7 | Sigma Aldrich |
| | | stearyl ether | | |
| 157. | Deceth-6 | Alfonic 1012-6.0 | 5168-89-8 | Sasol North |
| | | Ethoxylate | | America, Inc. |
| 158. | Steareth-6 | Emalex 606 | 2420-29-3 | Nihon Emulsion |
| | | | | Company, Ltd. |
| 159. | Hexaethylene glycol | Hexaethylene glycol | 3055-96-7 | Sigma-Aldrich |
| | monododecyl ether | monododecyl ether | | (UK) |
| 160. | Hexaethylene glycol | Hexaethylene glycol | 5168-91-2 | Sigma-Aldrich |
| | monohexadecyl ether | monohexadecyl ether | | (UK) |
| 161. | Beheneth-5 | Nikkol BB-5 | 136207-49-3 | Nikko Chemicals |
| | | | | Co., Ltd. |
| 162. | Myreth-5 | Isoxal 12 | 92669-01-7 | Vevy Europe SpA |
| 163. | Steareth-5 | Jeecol SA-5 | 71093-13-5 | Jeen |
| | | | | International |
| | | | | Corporation |
| 164. | Ceteth-5 | Emalex 105 | 4478-97-1 | Nihon Emulsion |
| | | | | Company, Ltd. |
| 165. | Oleth-5 | Brij ® O5 | 5353-27-5 | Croda, Inc. |
| 166. | Laureth-5 | Safol ® 23E5 Ethoxylate | 3055-95-6 | Sasol North |
| | | | | America, Inc. |
| 167. | Steareth-4 | Jeecol SA-4 | 59970-10-4 | Jeen |
| | | | | International |
| | | | | Corporation |
| 168. | Laureth-4 | Brij ® L4 | 5274-68-0 | Croda, Inc. |
| 169. | Myreth-4 | Homulgator 920 G | 39034-24-7 | Grau Aromatics |
| | | | | GmbH & Company KG |
| 170. | Ceteth-4 | Procol CA-4 | 5274-63-5 | Protameen |
| | | | | Chemicals |
| 171. | Oleth-4 | Chemal OA-4 | 5353-26-4 | Chemax, Inc. |
| 172. | Oleth-4 | Chemal OA-4 | 103622-85-1 | Chemax, Inc. |
| 173. | Polyimide-1 | Aquaflex ™ XL-30 | 497926-97-3 | Chemwill |
| 174. | Polymethoxy | Caswell No. 494CA | 56709-13-8 | Angene Chemical |
| | Bicyclic Oxazolidine | | | |
| 175. | Hydroxymethyl | Zoldine ™ ZT | 6542-37-6 | Angus Chemical |
| | Dioxoazabicyclooctane | | | Company |
| 176. | Dihydro-7a- | 5-Ethyl-1-aza-3,7- | 7747-35-5 | Sigma Aldrich |
| | ethyloxazolo[3,4-c]oxazole | dioxabicyclo[3.3.0]octane | | (UK) |
| 177. | Dibenzylidene Sorbitol | Disorbene ® | 32647-67-9 | Roquette |
| | | | | America, Inc. |
| 178. | Dimethyldibenzylidene | Millad ® 3988 | 135861-56-2 | Milliken |
| | Sorbitol | | | Chemicals |
| 179. | Laureth-2 | Alfonic 1216CO-2 | 3055-93-4 | Sasol North |
| | | Ethoxylate | | America, Inc. |
| 180. | 2-(2-Butoxyethoxy)ethyl | Piperonyl Butoxide | 51-03-6 | Sigma-Aldrich |
| | (6-propylpiperonyl) ether | | | (UK) |
| 181. | Menthone Glycerin Acetal | Frescolat ® MGA | 63187-91-7 | Symrise |
| 182. | Propylene Glycol Caprylate | Mackaderm PGC | 68332-79-6 | Rhodia Inc. |
| 183. | Diethoxynonadiene | SBB016951 | 67674-36-6 | Ambinter |
| 184. | Menthoxypropanediol | Coolact ® 10 | 87061-04-9 | Takasago |
| | Alcohols | | | International |
| | | | | Corporation |
| 185. | 2-Diphenylmethoxy- | Diphenhydramine HCl | 147-24-0 | Sigma-Aldrich |
| | N,N-dimethylethylamine | | | (UK) |
| | hydrochloride | | | |
| 186. | 3-((2- | — | 70445-33-9 | — |
| | ethylhexyl)oxy)propane- | | | |
| | 1,2-diol | | | |
| 187. | 3-((2- | — | — | — |
| | propylheptyl)oxy)propane- | | | |
| | 1,2-diol | | | |
| 188. | 1-amino-3-((2- | — | 99509-00-9 | — |
| | ethylhexyl)oxy)propan-2-ol | | | |
| 189. | 1-(1-Methyl-2- | Di(propylene glycol) | 29911-27-1 | Sigma Aldrich |
| | propoxyethoxy)-2-propanol | propyl ether | | (UK) |

According to some examples, the modulator(s) can be characterized as being “low odor”, “substantially non-odorous”, or non-odorous. In some examples, if the modulator is present at 1 wt % or less, no odor may be detected from the modulator

In some examples, the fragrance modulator is biodegradable. This can make the fragrance composition to which it is included a “green” or environmentally friendly fragrance composition.

The fragrance modulator component can be present in an amount of from about 0.1 wt % to about 27 wt % relative to the total weight of the composition of the composition, about 0.5 wt % to about 18 wt %, about 2.5 wt % to about 15 wt %, or less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 wt %. If there are more than one fragrance modulators, then the ranges provided hereinabove cover the total of all of the fragrance modulators.

Fragrance modulators employed can be a liquid at temperatures lower than 100° C., such as at ambient temperature. The fragrance modulators may be fully miscible with the fragrance materials to form a single phase liquid. However, if the fragrance materials are not entirely miscible, or are immiscible, then co-solvents (e.g., dipropylene glycol (DPG), triethyl citrate, or others well known to those skilled in the art) can be added to aid in the solubility of the fragrance materials.

According to various examples, the effect of the fragrance modulator on the fragrance profile, particularly the characters of the fragrance profile which is attributable to the high and moderate volatile fragrance materials, can be improved. By “improved” it is meant that the fragrance profile of the composition, particularly the components contributed by at least one of the high and moderate volatile fragrance materials, can be perceived by a panel of experts or professional evaluators or individual experts or professional evaluators at later time points such as, for example, 15 mins, 30 mins, 1 hour, 2 hours, 3 hours, 4 hours, 5 hours, 6 hours, 7 hours, 8 hours, 10 hours, and possibly all the way up to 24 hrs after application as compared to controls, e.g., lacking any of the disclosed non-odorous fragrance modulators such as pentylene glycol, polycitronello or an equivalent traditional fragrance construction.

Alternatively, by “improved” it can mean that the perception, by a panel of experts or professional evaluators or individual experts or professional evaluators, of the fidelity of the fragrance profile contributed by the high and moderate volatile fragrance materials is markedly increased or enhanced as compared to the controls. “Increased” or “enhanced” means that a panel of experts or professional evaluators or individual experts or professional evaluators perceives the fragrance profile, preferably the characters attributable to the high and/or moderate volatile fragrance materials, of a composition as not changing from its initial impression or the changes are minimal from when the composition was first applied to when it dissipates. In other words, the fidelity of the perceived fragrance profile of the composition is maintained over time. In contrast the composition lacking any of the disclosed nom-odorous fragrance modulators or an equivalent traditional fragrance construction will undergo a rapid loss of the characters attributable to the high and/or moderate volatile fragrance materials.

Such a solution as presented herein provides enhanced or improved fidelity and/or longevity of the fragrance profile, particularly amongst those compositions formulated from volatile fragrance materials having moderate to high vapor pressure ranges (greater than or equal to 0.001 Torr (0.000133 kPa) at 25° C.), without having to rely on the presence or significant amounts of the low volatile fragrance materials, which has a tendency to overpower and alter the overall fragrance profile, particularly over time. As a result, the present disclosure provides the perfumer options to formulate compositions having new fragrance profiles not possible before.

Additionally, according to some embodiments, the perceived harshness of overdosing of the fragrance material is mitigated or absent, as compared to the same perception in a fragrance in the absence of the modulator.

### Volatile Solvents

The composition according to the present invention, can include a volatile solvent present in the amount of from about 20 wt % to about 99 wt % relative to the total weight of the composition, about 30 wt % to about 80 wt %, about 55 wt % to about 75 wt %, or less than, equal to, or greater than about 20 wt %, 25, 30, 35, 40, 45, 50, 55, 60, 65, 70, 75, 80, 85, 90, 95, or about 99 wt %, and wherein the solvent is a branch or unbranched C₁ to C₁₀ alkyl, alkenyl or alkynyl group having at least one alcohol moiety, preferably ethanol, or isopropanol, or other alcohols (e.g., methanol, propanol, isopropanol, butanol, and mixtures thereof) commonly found in commercial fine fragrance products.

Accordingly, ethanol may be present in any of the compositions of the present invention, and more specifically, it will form from about 5 wt % to about 95 wt %, or even from about 10 wt % to about 80 wt %, 25 wt % to about 75 wt % of the composition, or combinations thereof, relative to the total weight of the composition. Alternatively, ethanol may be present in an amount of from about 10 wt % or 25 wt % to about 75 wt % or 80 wt %, relative to the total weight of the composition. The ethanol useful in the present invention may be any acceptable quality of ethanol, compatible and safe for the specific intended use of the composition such as, for example, topical applications of fine fragrance or cosmetic compositions.

### Water

In some examples (e.g., those including a volatile solvent), water may be present in any of the compositions of the present invention, and more specifically, it may not exceed about 16 wt % relative to the total weight of the composition. It is understood that the amount of water present in the composition may be from the water present in the volatile solvent (e.g., ethanol) used in the composition, as the case may be.

### Non-Volatile Solvents:

The composition may comprise a non-volatile solvent or a mixture of non-volatile solvents. Non-limiting examples of non-volatile solvents include benzyl benzoate, diethyl phthalate, isopropyl myristate, propylene glycol, dipropylene glycol, triethyl citrate, and mixtures thereof. These solvents often are introduced to the product via the perfume oil as many perfume raw materials may be purchased as a dilution in one of these solvents. Where non-volatile solvents are present, introduced either with the perfume materials or separately, then for the purposes of calculating the proportion of fragrance component having a vapor pressure of less than 0.001 Torr (0.000133 kPa) at 25° C. the total fragrance components does not include non-volatile solvents. Where non-volatile solvents are present, introduced either with the perfume materials or separately, then for the purposes of calculating the total level of fragrance component this does not include non-volatile solvents. In addition, if present with cyclic oligosacchrides, the non-volatile solvent may be included at a weight ratio of the non-volatile solvent to the cyclic oligosaccharide of less than 1:1, less than 1:2, less than 1:10, or less than 1:100.

### Entrapment Materials

In other examples, compositions of the present invention can include an entrapment material at a level such that the weight ratio of the entrapment material to the fragrance materials is in the range of from about 1:20 to about 20:1. in some examples, the composition may comprise an entrapment material present in the amount of from about 0.001 wt % to about 40 wt %, from about 0.1 wt % to about 25 wt %, from about 0.3 wt % to about 20 wt %, from about 0.5 wt % to about 10 wt %, or from about 0.75 wt % to about 5 wt %, relative to the total weight of the composition. The compositions disclosed herein may include from 0.001 wt % to 40%, from 0.1 wt % to 25 wt %, from 0.3 wt % to 20 wt %, from 0.5 wt % to 10 wt % or from 0.75 wt % to 5 wt %, relative to the total weight of the composition, of a cyclic oligosaccharide.

Suitable entrapment materials for use herein are selected from polymers; capsules, microcapsules and nanocapsules; liposomes, absorbents; cyclic oligosaccharides and mixtures thereof. Preferred are absorbents and cyclic oligosaccharides and mixtures thereof. Highly preferred are cyclic oligosaccharides (see PCT Publication Nos. WO2000/67721 (Procter & Gamble); and WO2000/67720 (Procter & Gamble); and U.S. Pat. No. 6,893,647 (Procter & Gamble)).

As used herein, the term “cyclic oligosaccharide” means a cyclic structure comprising six or more saccharide units. Preferred for use herein are cyclic oligosaccharides having six, seven or eight saccharide units and mixtures thereof, more preferably six or seven saccharide units and even more preferably seven saccharide units. It is common in the art to abbreviate six, seven and eight membered cyclic oligosaccharides to α, β and γ respectively.

The cyclic oligosaccharide of the compositions used for the present invention may comprise any suitable saccharide or mixtures of saccharides. Examples of suitable saccharides include, but are not limited to, glucose, fructose, mannose, galactose, maltose and mixtures thereof. However, preferred for use herein are cyclic oligosaccharides of glucose. The preferred cyclic oligosaccharides for use herein are α-cyclodextrins or μ-cyclodextrins, or mixtures thereof, and the most preferred cyclic oligosaccharides for use herein are β-cyclodextrins.

The cyclic oligosaccharide, or mixture of cyclic oligosaccharides, for use herein may be substituted by any suitable substituent or mixture of substituents. Herein the use of the term “mixture of substituents” means that two or more different suitable substituents can be substituted onto one cyclic oligosaccharide. The derivatives of cyclodextrins consist mainly of molecules wherein some of the OH groups have been substituted. Suitable substituents include, but are not limited to, alkyl groups; hydroxyalkyl groups; dihydroxyalkyl groups; (hydroxyalkyl)alkylenyl bridging groups such as cyclodextrin glycerol ethers; aryl groups; maltosyl groups; allyl groups; benzyl groups; alkanoyl groups; cationic cyclodextrins such as those containing 2-hydroxy-3-(dimethylamino) propyl ether; quaternary ammonium groups; anionic cyclodextrins such as carboxyalkyl groups, sulphobutylether groups, sulphate groups, and succinylates; amphoteric cyclodextrins; and mixtures thereof.

The substituents may be saturated or unsaturated, straight or branched chain. Preferred substituents include saturated and straight chain alkyl groups, hydroxyalkyl groups and mixtures thereof. Preferred alkyl and hydroxyalkyl substituents are selected from C₁-C₈ alkyl or hydroxyalkyl groups or mixtures thereof, more preferred alkyl and hydroxyalkyl substituents are selected from C₁-C₆ alkyl or hydroxyalkyl groups or mixtures thereof, even more preferred alkyl and hydroxyalkyl substituents are selected from C₁-C₄ alkyl or hydroxyalkyl groups and mixtures thereof. Especially preferred alkyl and hydroxyalkyl substituents are propyl, ethyl and methyl, more especially hydroxypropyl and methyl and even more preferably methyl.

Suitable cyclic oligosaccharides for use in the present invention are unsubstituted, or are substituted by only saturated straight chain alkyl, or hydroxyalkyl substituents. Therefore, preferred examples of cyclic oligosaccharides for use herein are α-cyclodextrin, β-cyclodextrin, methyl-α-cyclodextrin, methyl-β-cyclodextrin, hydroxypropyl-α-cyclodextrin and hydroxypropyl-β-cyclodextrin. Most preferred examples of cyclic oligosaccharides for use herein are methyl-α-cyclodextrin and methyl-β-cyclodextrin. These are available from Wacker-Chemie GmbH Hanns-Seidel-Platz 4, Munchen, DE under the tradename Alpha W6 M and Beta W7 M respectively.

The cyclic oligosaccharides of the compositions used for the present invention can be soluble in water, ethanol, or both water and ethanol. As used herein “soluble” means at least about 0.1 g of solute dissolves in 100 mL of solvent, at 25° C. and 1 standard atmospheric pressure (760 mmHg). The cyclic oligosaccharides for use herein have a solubility of at least about 1 g/100 mL, at 25° C. and 1 atm of pressure. In some examples, cyclic oligosaccharides are only present at levels up to their solubility limits in a given composition at room temperature. A person skilled in the art will recognize that the levels of cyclic oligosaccharides used in the present invention will also be dependent on the components of the composition and their levels, for example the solvents used or the exact fragrance oils, or combination of fragrance oils, present in the composition. Therefore, although the limits stated for the entrapment material are preferred, they are not exhaustive.

### Propellants

The compositions described herein may include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42% by weight of the total fill of materials stored within the container.

### Antiperspirant Active

The compositions described herein may be free of, substantially free of, or may include an antiperspirant active (e.g., any substance, mixture, or other material having antiperspirant activity). Examples of antiperspirant actives include astringent metallic salts, like the inorganic and organic salts of aluminum, zirconium and zinc, as well as mixtures thereof. Such antiperspirant actives include, for example, the aluminum and zirconium salts, such as aluminum halides, aluminum hydroxyhalides, zirconyl oxyhalides, zirconyl hydroxyhalides, and mixtures thereof.

### Other Ingredients

In yet another aspect, the composition consists essentially of the recited ingredients but may contain small amounts (not more than about 10 wt %, preferably no more than 5 wt %, or preferably no more than 2 wt % thereof, relative to the total weight of the composition) of other ingredients that do not impact on the fragrance profile, particularly the evaporation rate and release of the fragrance materials. For example, a fine fragrance composition may comprise stabilizing or anti-oxidant agents, UV filters or quenchers, or colouring agents, commonly used in perfumery. There are a number of other examples of additional ingredients that are suitable for inclusion in the present compositions, particularly in compositions for cosmetic use. These include, but are not limited to, alcohol denaturants such as denatonium benzoate; UV stabilizers such as benzophenone-2; antioxidants such as tocopheryl acetate; preservatives such as phenoxyethanol, benzyl alcohol, methyl paraben, and propyl paraben; dyes; pH adjusting agents such as lactic acid, citric acid, sodium citrate, succinic acid, phosphoric acid, sodium hydroxide, and sodium carbonate; deodorants and anti-microbials such as farnesol and zinc phenolsulphonate; humectants such as glycerine; oils; skin conditioning agents such as allantoin; cooling agents such as trimethyl isopropyl butanamide and menthol; silicones; solvents such as hexylene glycol; hair-hold polymers such as those described in PCT Publication No. WO94/08557 (Procter & Gamble); salts in general, such as potassium acetate and sodium chloride and mixtures thereof.

In yet another aspect, the composition of the present invention, depending on its intended use, is a mixture of fragrance materials possibly together with other ingredients such as, for example, perfume carriers. By the term “perfume carrier”, it is meant to include materials which are practically neutral from a perfumery point of view, e.g., which does not significantly alter the organoleptic properties of perfuming components. The perfume carrier may be a compatible liquid or solid fillers, diluents, and the like. The term “compatible”, as used herein, means that the components of the compositions of this invention are capable of being combined with the primary actives of the present invention, and with each other, in a manner such that there is no interaction which would substantially reduce the efficacy of the composition under ordinary use situations. The type of carrier utilized in the present invention depends on the type of product desired and may comprise, but are not limited to, solutions, aerosols, emulsions (including oil-in-water or water-in-oil), gels, and liposomes. Preferably, the carrier is a liquid and will be a solvent such as, for example, dipropyleneglycol, diethyl phthalate, isopropyl myristate, benzyl benzoate, 2-(2-ethoxyethoxy)-1-ethanol, or ethyl citrate (triethyl citrate).

In yet another aspect, the compositions for use in the present invention may take any form suitable for use, such as for perfumery or cosmetic use. These include, but are not limited to, vapor sprays, aerosols, emulsions, lotions, liquids, creams, gels, sticks, ointments, pastes, mousses, powders, granular products, substrates, cosmetics (e.g., semi-solid or liquid makeup, including foundations) and the like. In some examples, the compositions for use in the present invention take the form of a vapor spray. Compositions of the present invention can be further added as an ingredient to other compositions, preferably fine fragrance or cosmetic compositions, in which they are compatible. As such they can be used within solid composition or applied substrates etc. Examples of products including the composition can include a fabric care product, an air care product, a home care
product, a beauty care product, or a mixture thereof. Specific examples of products can include a perfume, an eau de toilette, an eau de parfum, a cologne, a body splash, a lotion, a cream, a shampoo, a conditioner, a hair mist, a body oil, a deodorant, a solid fragrance, or a body spray. The composition can be contacted with skin, hair, or a fabric.

### Smart Gels

For some embodiments, fragrance formulations include one or more polyurethanes such as polyurethane-1, one or more modulators and a smart gel such as ExpertGel. A smart gel is liquid at room temperature and gels higher temperatures. The gel entraps fragrance and prevents evaporation and skin penetration. The smart gels include ExpertGel EG 312 and EG 412, both of which are made by DKSH France S.A. The ExpertGel is described in U.S. Pat. No. 7,339,013. The ExpertGel includes polymer chains of terpolymer type which are constituted by poly(ethylene oxide) (PEO) and poly(propylene oxide) (PPO) of PEO-PPO-PEO form, which are modified at their termini by groups which can essentially be other chains of PEO-PPO-PEO, acid segments, amine groups or PEOs, these chains being linked to the terpolymer chains via chemical bridges which are constituted by urethane bridges, urea bridges, allophanate bridges and biuret bridges, and which include more than 80% by weight of said PEO-PPO-PEO.

### Article of Manufacture

The composition may be included in an article of manufacture comprising a spray dispenser. The spray dispenser may comprise a vessel for containing the composition to be dispensed. The spray dispenser may comprise an aerosolized composition (e.g., a composition comprising a propellant) within the vessel as well. Other non-limiting examples of spray dispensers include non-aerosol dispensers (e.g., vapor sprays), manually activated dispensers, pump-spray dispensers, or any other suitable spray dispenser available in the art.

### Methods of Using the Compositions

The composition of the present invention according to any embodiments described herein is a useful perfuming composition, which can be advantageously used as consumer products intended to perfume any suitable substrate. As used herein, the term “substrate” means any surface to which the composition of the present invention may be applied to without causing any undue adverse effect. For example, this can include a wide range of surfaces including human or animal skin or hair, paper (fragranced paper), air in a room (air freshener or aromatherapy composition), fabric, furnishings, dishes, hard surfaces and related materials. Preferred substrates include body surfaces such as, for example, hair and skin, most preferably skin.

The composition of the present invention may be used in a conventional manner for fragrancing a substrate. An effective amount of the composition, such as from about 1 μL to about 100 mL, preferably from about 10 μL to about 1,000 μL, more preferably from about 25 μL to about 500 μL, from about 50 μL to about 100 μL, from about 100 μL to about 20 mL, or combinations thereof, is applied to the suitable substrate. Alternatively, an effective amount of the composition of the present invention is less than, equal to, or greater than about 1 μL, 10 μL, 25 μL or 50 μL to about 100 μL, 500 μL, 1,000 μL, 10,000 μL, 10 mL, 20 mL, 25 mL, 30 mL, 40 mL, 50 mL, 60 mL, 70 mL, 80 mL, 90 mL, or 100 mL. The composition may be applied by hand or applied utilizing a delivery apparatus such as, for example, vaporizer or atomizer. Preferably, the composition is allowed to dry after its application to the substrate. The scope of the present invention should be considered to cover one or more distinct applications of the composition or the continuous release of a composition via a vaporizer or other type of atomizer.

The present disclosure provides a method for imparting, intensifying, or modifying an odor on human skin or human hair, comprising applying to human skin and/or human hair the composition of the present invention. Examples of notes or characters that can be enhanced include any of those of: citrus-type note, green-type note, watery-type notes, aromatic-type notes, herbal-type notes, mint-type notes, lavender-type notes, rosemary-type notes, spicy-type notes, cinnamon-type notes, clove-type notes, pepper-type notes, cumin-type notes, ginger-type notes, fougere-type note, patchouli-type notes, floral-type notes, gourmand-type notes, sweet-type notes, vanilla-type notes, amber-type notes, woody-type notes, cedarwood-type notes, sandalwood type notes, vetyver-type notes and mixtures thereof.

Preferably, the fragrance profile or character of the composition of the present invention is detectable by a panel of experts or professional evaluators or individual experts or professional evaluators at later time points such as, for example, 15 mins, 30 mins, 1 hour, 2 hours, 3 hours, 4 hours, 5 hours, 6 hours, 7 hours, 8 hours, 10 hours, and possibly all the way up to 24 hours after application of the composition to a substrate as compared to controls (e.g., those without modulators).

In another aspect, the present invention is also directed to a method of producing a consumer product comprising bringing into contact or mixing into the product an organoleptically active quantity of a composition of the present invention.

Various embodiments of the present invention can be better understood by reference to the following Examples which are offered by way of illustration. The present invention is not limited to the Examples given herein.

### Analytical Evaporation Test

### Protocol

The following test is carried out to demonstrate the improved or enhanced longevity of a fragrance profile of a composition vs. a control. In particular, the test measures the effect of a fragrance modulator on the evaporation rate of one or more fragrance materials formulated in a composition. The evaporation response of the fragrance materials to the modulator, as a function of time, is measured using gas chromatography (“GC”).

1. A test composition may comprise a fragrance modulator with either: (i) a fragrance material or (ii) a blend of fragrance materials. The test compositions also contain ethanol, and deionized water. All the ingredients are admixed until evenly distributed in the test compositions.
2. A control composition to the test composition described in 1 above, is made in a similar manner to Step 1, except that the modulator is replaced by Glucam™ P-20 (alternatively referred to a “glucam” a propoxylated methyl glucose ether) or water.
3. An internal standard is needed to correct for variations of the amount of composition dispensed in the evaporation test, as well as loss during the GC analysis. The type of internal standard, its concentration or the step at which it is added to the fragrance material or compositions (test and control) must be selected appropriately by someone skilled in the art. Suitable non-limiting examples of internal standards are triethyl citrate or denatonium benzoate. The resultant solution is used in subsequent steps.
4. A hotplate is set to a temperature of 32° C. An aluminum container, such as TA Instruments T-Zero™ pan, is placed on the hotplate. 20 μL of the test or control composition is introduced in the aluminum container using a micropipette. Alternatively, the aluminum container may be filled with the test or control composition to its full capacity. The time at which this takes place is determined to be time zero (i.e., T=0). Multiple aluminum containers are prepared and left at the set temperature for pre-determined periods of time, such as for example 15 mins, 30 mins, 1 hr, 2 hrs, 3 hrs, 4 hrs, 5 hrs, 6 hrs, 8 hrs and up to 12 hrs.
5. The aluminum container is removed from the hotplate at the end of the pre-determined time and transferred by being inserted into a 4 mL glass vial already containing at least 2 mL of highly volatile solvent, such as high purity ethanol or hexane.
6. The glass vial is mixed to extract the fragrance materials into the solvent phase. 1 mL of the resultant solution is transferred to a 2 mL GC vial.
7. The GC vial is analysed on an Agilent GC system 7890 equipped with an autosampler, or equivalent. A GC column such as DB-5MS, DB-1MS models or equivalent phases, is used. Gas chromatography with flame ionization detection (“FID”) or with mass spectrometry (“MS”) can be used for the identification and quantification of fragrance material in the compositions. The column dimensions as well as GC settings, FID gas flows and temperature or MS parameters, must be adjusted to optimize the detection and response of the fragrance material and internal standard being monitored
8. The peak area of the fragrance material and internal standard are recorded. The peak area ratio of the fragrance material and the internal standard is calculated at each time point for each sample composition. The % of non-evaporated fragrance material remaining from T=0 is calculated at each time point for each sample composition. This is done for both the test and control compositions. Significance is determined by comparison of the evaporation profile for the same fragrance material or same fragrance mixture in the test and control compositions.

The constituents of the test compositions are shown in Table 6. The compositions referred to in Table 6 as “glucam”, “citropol”, and “hydrolyte 5” include performance data in Tables 7-16.

TABLE 6

| | % w/w | | |
|---|---|---|---|
| Ingredient | Glucam | Citropol | Hydrolite 5 |
| Fragrance mixture | 7 | 7 | 7 |
| Ethanol 96% | 73 | 78 | 78 |
| Water | 5 | 5 | 5 |
| Glucam ™ P20 | 15 | — | — |
| Citropol | — | 10 | — |
| Hydrolite 5 | — | — | 10 |

### Results

Tables 7-11 show the amount of a fragrance material in a fragrance mixture (containing at least dihydro mycenol, linalool, dimethyl benzyl carbionol, alpha-terpineol, and carvone) retained after various time points (0 minutes, 30 minutes, 60 minutes, 180 minutes, and 360 minutes), in a Glucam™ P20 control composition and a pentylene glycol (“hydrolite 5”) test composition, respectively.

TABLE 7

| | CAS 18479-58-8 | |
|---|---|---|
| Dihydro Myrcenol | Glucam Average (n = 3) | Hydrolite 5 (n = 1) |
| Time (min) | % Amount | % Amount |
| 0 | 100.0 | 100.0 |
| 30 | 55.7 | 76.0 |
| 60 | 33.5 | 48.1 |
| 180 | 11.1 | 8.2 |
| 360 | 0.3 | 5.0 |

TABLE 8

| | CAS 78-70-6 | |
|---|---|---|
| Linalool | Glucam Average (n = 3) | Hydrolite 5 (n = 1) |
| Time (min) | % Amount | % Amount |
| 0 | 100.0 | 100.0 |
| 30 | 54.6 | 70.1 |
| 60 | 38.1 | 45.9 |
| 180 | 13.1 | 6.9 |
| 360 | 0.6 | 4.0 |

TABLE 9

| Dimethyl | CAS 100-86-7 | |
|---|---|---|
| Benzyl Carbinol | Glucam Average (n = 3) | Hydrolite 5 (n = 1) |
| Time (min) | % Amount | % Amount |
| 0 | 100.0 | 100.0 |
| 30 | 80.5 | 93.2 |
| 60 | 61.6 | 64.0 |
| 180 | 35.8 | 20.5 |
| 360 | 8.2 | 13.2 |

TABLE 10

| | CAS 98-55-5 | |
|---|---|---|
| Alpha-Terpineol | Glucam Average (n = 3) | Hydrolite 5 (n = 1) |
| Time (min) | % Amount | % Amount |
| 0 | 100.0 | 100.0 |
| 30 | 77.1 | 85.6 |
| 60 | 63.7 | 67.3 |
| 180 | 35.3 | 26.6 |
| 360 | 7.2 | 18.6 |

TABLE 11

| | CAS 2244-16-8 | |
|---|---|---|
| Carvone | Glucam Average (n = 3) | Hydrolite 5 (n = 1) |
| Time (min) | % Amount | % Amount |
| 0 | 100.0 | 100.0 |
| 30 | 72.3 | 81.7 |
| 60 | 52.7 | 48.9 |
| 180 | 26.0 | 8.4 |
| 360 | 1.4 | 5.0 |

Tables 12-16 show the amount of certain fragrance materials in a fragrance mixture (containing at least dihydro mycenol, linalool, dimethyl benzyl carbionol, alpha-terpineol, and carvone) retained after various time points (0 minutes, 30 minutes, 60 minutes, 120 minutes, 180 minutes, and 360 minutes), in a Glucam™ P20 control composition and a polycitropol test composition, respectively.

TABLE 12

| | CAS 18479-58-8 | |
|---|---|---|
| Dihydro Myrcenol | Glucam Average (n = 3) | Citropol Average (n = 3) |
| Time (min) | % Amount | % Amount |
| 0 | 100.0 | 100.0 |
| 30 | 55.7 | 69.6 |
| 60 | 33.5 | 63.1 |
| 180 | 11.1 | 27.8 |
| 360 | 0.3 | 18.7 |

TABLE 13

| | CAS 78-70-6 | |
|---|---|---|
| Linalool | Glucam Average (n = 3) | Citropol Average (n = 3) |
| Time (min) | % Amount | % Amount |
| 0 | 100.0 | 100.0 |
| 30 | 54.6 | 70.0 |
| 60 | 38.1 | 66.8 |
| 180 | 13.1 | 32.4 |
| 360 | 0.6 | 16.6 |

TABLE 14

| Dimethyl Benzyl | CAS 100-86-7 | |
|---|---|---|
| Carbinol | Glucam Average (n = 3) | Citropol Average (n = 3) |
| Time (min) | % Amount | % Amount |
| 0 | 100.0 | 100.0 |
| 30 | 80.5 | 85.8 |
| 60 | 61.6 | 88.4 |
| 180 | 35.8 | 55.6 |
| 360 | 8.2 | 49.6 |

TABLE 15

| | CAS 98-55-5 | |
|---|---|---|
| Alpha-Terpineol | Glucam Average (n = 3) | Citropol Average (n = 3) |
| Time (min) | % Amount | % Amount |
| 0 | 100.0 | 100.0 |
| 30 | 77.1 | 82.3 |
| 60 | 63.7 | 84.1 |
| 180 | 35.3 | 59.5 |
| 360 | 7.2 | 56.0 |

TABLE 16

| | CAS 2244-16-8 | |
|---|---|---|
| Carvone | Glucam Average (n = 3) | Citropol Average (n = 3) |
| Time (min) | % Amount | % Amount |
| 0 | 100.0 | 100.0 |
| 30 | 72.3 | 79.8 |
| 60 | 52.7 | 77.7 |
| 180 | 26.0 | 48.1 |
| 360 | 1.4 | 42.8 |

The results show that the Citropol and pentylene glycol modulators can help to retain a fragrance composition longer than a glucam modulator. It has also been found that citropol and polyurethane-64 produce a synergistic effect in retaining a fragrance composition. A combination of pentylene glycol and polyurethane-64 also produces a synergistic effect in retaining a fragrance composition.

Formulation embodiments that include Polyurethane-64 include the following:

### Example 1: Body Mist

| Phase A | Alcohol | ad 100 |
|---|---|---|
| | Fragrance | 4.5 |
| Phase B | Water | 16 |
| | White Ginger Extract | 0.1 |
| Phase C | Alcohol | 1.5 |
| | Polyurethane-64 | 0.5 |
| Phase D | Colorant | 0.2 |

Mix alcohol and fragrance with agitation. Add phase B with agitation and mix until clear and uniform. Filter the main batch and add separate mixed phase C to the main batch with agitation. Add phase D and mix until uniform.

### Example 2: Perfume

| Ingredient | Wt % |
|---|---|
| Phase A | |
| Alcohol denat | up to 100 |
| Water | 3.364 |
| Parfum | 20.000 |
| Butyl | 0.325 |
| Methoxydibenzoylmethane | |
| Ethylhexyl methoxycinnamete | 0.845 |
| Octocrylene | 0.130 |
| Phase B | |
| Polyurethane-64 | 0.240 |

Mix all Phase A ingredients until full homogenization. Cool down and filter the main batch. Add Phase B with stirring.

### Example 3: Perfume

| | % |
|---|---|
| Phase A | |
| Alcohol denat | up to 100 |
| Parfum | 5.510 |
| Ethylhexyl | 0.600 |
| methoxycinnamate | |
| Butyl | 0.100 |
| Methoxydibenzoylmethane | |
| Benzophenone-3 | 0.150 |
| Ethylhexyl Salicylate | 0.150 |
| Water | 15.589 |
| Disodium EDTA | 0.001 |
| Phase B | |
| Polyurethane-64 | 0.240 |

Mix all Phase A ingredients until full homogenization. Cool down and filter the main batch. Add Phase B with stirring.
Mix all Phase A ingredients until full homogenization. Cool down and filter the main batch. Add Phase B with stirring.

### Example 4: Perfume

| INCI | Wt % |
|---|---|
| Phase A | |
| Alcohol denat | up to 100 |
| Parfum | 5.250 |
| Ethylhexyl | 0.878 |
| methoxycinnamate | |
| Butyl | 0.338 |
| Methoxydibenzoylmethane | |
| Octocrylene | 0.135 |
| Aqua | 15.230 |
| Disodium EDTA | 0.001 |
| Dipropylene Glycol | 3.570 |
| Phase B | |
| Polyurethane-64 | 0.400 |

Mix all Phase A ingredients until full homogenization. Cool down and filter the main batch. Add Phase B with stirring.
Mix all Phase A ingredients until full homogenization. Cool down and filter the main batch. Add Phase B with stirring.

### Example 5: Perfume

| INCI | Wt % |
|---|---|
| Phase A | |
| Alcohol denat | up to 100 |
| Parfum | 5.250 |
| Ethylhexyl | 0.878 |
| methoxycinnamate | |
| Butyl | 0.338 |
| Methoxydibenzoylmethane | |
| Octocrylene | 0.135 |
| Aqua | 15.230 |
| Disodium EDTA | 0.001 |
| Pentylene Glycol | 3.570 |
| Phase B | |
| Polyurethane-64 | 0.400 |

Mix all Phase A ingredients until full homogenization. Cool down and filter the main batch. Add Phase B with stirring.

### Example 6: Perfume

| INCI | Wt % |
|---|---|
| Phase A | |
| Alcohol denat | up to 100 |
| Parfum | 5.250 |
| Ethylhexyl | 0.878 |
| methoxycinnamate | |
| Butyl | 0.338 |
| Methoxydibenzoylmethane | |
| Octocrylene | 0.135 |
| Aqua | 15.230 |
| Disodium EDTA | 0.001 |
| PPG-20 Methyl Glucose | 3.570 |
| Ether | |
| Phase B | |
| Polyurethane-64 | 0.400 |

Mix all Phase A ingredients until full homogenization. Cool down and filter the main batch. Add Phase B with stirring.

### Example 7: Perfume

| | % |
|---|---|
| Phase A | |
| Alcohol denat | up to 100 |
| Parfum | 10.000 |
| Ethylhexyl | 1.200 |
| methoxycinnamate | |
| Butyl | 0.200 |
| Methoxydibenzoylmethane | |
| Benzophenone-3 | 0.300 |
| Ethylhexyl Salicylate | 0.300 |
| Water | 10.120 |
| Phase B | |
| Polyurethane-64 | 0.600 |

From the foregoing description, one skilled in the art can easily ascertain the essential characteristics of this invention and, without departing from the spirit and scope thereof, can make various changes and modifications of the invention to adapt it to various usages and conditions.

TABLE 17

| Odor Intensity Scale | |
|---|---|
| Score | Fragrance intensity |
| 0 | None |
| 1 | Very Weak |
| 2 | Weak |
| 3 | Moderate |
| 4 | Strong |
| 5 | Very Strong |

Exemplary fragrance formulations were evaluated for a degree of odor detected by olfactive experts. Fragrance samples containing 1% polyurethane-64 had odors that were stronger and more intense.

### Olfactive Evaluation

Comparison between sample from examples 2 and 7 and the same formulation without Baycusan C2000 after 3 h—blind test with olfactive experts

| | Evaluator A | Evaluator B | Evaluator C |
|---|---|---|---|
| Example 2 | | | |
| Sample without | 3 | 3 | 4 |
| Polyurethane- | | | |
| 64 | | | |
| Sample with | 4 - Slightly more floral | 5 - slightly | 5 -stronger |
| 1.0% | indolic, more powerful | stronger and | intensity |
| Polyurethane- | | juicier | |
| 64 | | | |
| Example 7 | | | |
| Sample without | 4 | 4 | 2 |
| Polyurethane- | | | |
| 64 | | | |
| Sample with | 5 - More smoky/ | 5 - Stronger | 4 - More |
| 1.0% | slightly more minty | | violete (ionones), |
| Polyurethane- | | | higher intensity |
| 64 | | | |

Comparison between sample from examples 3 and 4 and the same formulation without Baycusan C2000 after stability—blind test with olfactive experts

| | Evaluator A | Evaluator B | Evaluator C |
|---|---|---|---|
| Example 3 | | | |
| Sample without | 2 | 2 | 2 |
| Polyurethane- | | | |
| 64 | | | |
| Sample with | 3 - Slightly fresher | 4 - Sharp green | 4 - Stronger |
| 1.0% | | note | intensity |
| Polyurethane- | | | |
| 64 | | | |
| Example 4 | | | |
| Sample without | 2 | 3 | 3 |
| Polyurethane- | | | |
| 64 | | | |
| Sample with | 3 - Better, more | 4 - Less oxidized | 4 |
| 1.0% | woody | | |
| Polyurethane- | | | |
| 64 | | | |

According to the experts, the formulation embodiments that include polyurethane-64 are perceived stronger and less oxidized after stability.

### Analytical Fixative Test

The following test demonstrated an improved or enhanced retention of a fragrance of a composition of the present invention for formulations that include polyurethane-64. In particular, the test measured the film forming and fixative effect on fragrance materials of a composition applied to an in-vitro substrate (e.g., glass slide). The properties were triggered after exposure to water. The amount of fragrance materials left on the substrate was recovered with a solvent and analyzed using gas chromatography coupled with a Flame Ionisation Detector (FID) or mass spectrometry (MS).

1. A test composition included ethanol, water, one or more fragrances and a high molecular weight ingredient with at least one carbamate linkage.
2. A control composition to the test composition described in step 1 above was made with ethanol, water, and the same one of more fragrances used in the test composition.
3. A hotplate was set to a temperature of 32° C. A glass slide, with dimensions of about 76×26 mm, was placed on the hotplate. 50 uL of the test or control composition was dispensed on a test area of the glass slide using a micropipette. The time at which this took place was determined to be time zero (i.e., T=0).
4. The solution was left to evaporate on the glass slide at 32° C. for 10 min from T=0. The glass slide was then immersed in water having a temperature around 20-27° C. for 4 minutes. The excess water was dabbed carefully avoiding the test area, and the glass slide was left to further dry for 10 mins at 32° C. on a hotplate.
5. The test area was rinsed with a known volume of solvent that solubilized the fragrance residue completely. Examples included ethanol, hexane, or dichloromethane. The resulting solution was collected in a container, or directly transferred to a 2 mL GC vial for analysis.
6. Steps 3 and 4 were repeated on a different slide, after 10 mins and 2 hrs of evaporation, followed by step 5 at the 2 hr timepoint.
7. Steps 3 and 4 were repeated on a different slide, after 10 mins, 2 hrs and 4 hrs of evaporation, followed by step 5 at the 4 hr timepoint.
8. The total or individual peak area of the fragrance materials were recorded at each timepoint. Significance is determined by comparison of the peak area for the same fragrance material or same fragrance mixture in the test and control compositions.
Results for formulations presented below are shown graphically in FIG. 3 and FIG. 4.

### Example 8

| | % |
|---|---|
| Phase A | |
| Alcohol denat | up to 100 |
| Parfum | 10.000 |
| Ethylhexyl | 0.300 |
| Methoxycinnamate (and) | |
| Diethylamino | |
| Hydroxybenzoyl Hexyl | |
| Benzoate | |
| Water | 15.000 |
| Phase B | |
| Polyurethane-64 | 0.000 |

### Example 9

| | % |
|---|---|
| Phase A | |
| Alcohol denat | up to 100 |
| Parfum | 10.000 |
| Ethylhexyl | 0.300 |
| Methoxycinnamate (and) | |
| Diethylamino | |
| Hydroxybenzoyl Hexyl | |
| Benzoate | |
| Water | 15.000 |
| Phase B | |
| Polyurethane-64 | 0.400 |

### Example 10

| | % |
|---|---|
| Phase A | |
| Alcohol denat | up to 100 |
| Parfum | 10.000 |
| Ethylhexyl | 0.300 |
| Methoxycinnamate (and) | |
| Diethylamino | |
| Hydroxybenzoyl Hexyl | |
| Benzoate | |
| Water | 15.000 |
| Phase B | |
| Polyurethane-64 | 1.200 |

### Olfactive Evaluation by Experts:

The steps 1 to 4 of the analytical fixative test were repeated for an olfactive evaluation. Instead of rinsing the residue left after immersion in water, the samples were evaluated by experts.

The same method was repeated in an in-vivo substrate (e.g. skin) with similar results.

TABLE 18

| Odor Intensity Scale | |
|---|---|
| Score | Fragrance intensity |
| +2 | Very intense vs the reference |
| +1 | More intense vs the reference |
| 0 | Similar to the reference |
| −1 | Weaker vs the reference |
| −2 | Very week vs the reference |

| | Evaluator A | Evaluator B | Evaluator C |
|---|---|---|---|
| Example 9 | | | |
| T = 4 hours | 0 | +1 | +2 |
| T = 10 min | +1 | +2 | +1 |
| Example 10 | | | |
| T = 4 hours | +1 | +2 | +2 |
| T = 10 min | +2 | +2 | +1 |

According to the experts, the formulation embodiments that include polyurethane-64 are perceived more intense after immersion in water. This supports that the fragrance residues from analytical data in FIGS. 3 and 4 is perceptible by human nose, concluding that the fragrances containing polyurethane-64 are water resistant.

### Fixative/Film Former Protocol Follow on Three Different EDT Formulations

TABLE 19

GC Total Fragrance Peak Area

| | | | | |
|---|---|---|---|---|
| Time (mins) | 0 | 10 | 120 | 240 |
| EDT without technology | 2896 | 422 | 89 | 58 |
| Example 10 | | 1192 | 784 | 621 |

### Repeatability Tests Following Below Protocol

50 uL on glass slide, evaporation for 10 mins, immersion in water for 4 mins+evaporation up to 2 hrs, immersion in water for 4 mins+evaporation up to 4 hrs, drying for 10 mins, rinsing with Ethanol and analysis by GCMS. Carried out in triplicate. Done with EDT from Example 9 and 10.

TABLE 20

| | GC Total Fragrance Peak Area | | |
|---|---|---|---|
| | Example | Example | Example |
| | 8 | 9 | 10 |
| Average (Error bar = +/−1SD) | 64 | 267 | 778 |
| Std deviation (n = 3) | 26 | 161 | 288 |

### Decrease of Fragrance Oil

The following test demonstrated an enhanced retention of a fragrance of a composition of the present invention for formulations that include polyurethane-64. In particular, the test measured the intensity of a composition applied to an in-vitro substrate (e.g., glass slide).

A test composition included ethanol, water, one or more fragrances and a high molecular weight ingredient with at least one carbamate linkage.

2. A control composition to the test composition described in step 1 above was made with ethanol, water, and the same one of more fragrances used in the test composition.
3. A hotplate was set to a temperature of 32° C. A glass slide, with dimensions of about 76×26 mm, was placed on the hotplate. 20 uL of the test or control composition was dispensed on a test area of the glass slide using a micropipette. The time at which this took place was determined to be time zero (i.e., T=0).
4. The solution was left to evaporate on the glass slide at 32° C. for 10 min from T=0 (top notes) and for 3 hours (base notes). The results are described in table 11 and graphically in FIG. 1 and FIG. 2.

### Example 11

| | % |
|---|---|
| Phase A | |
| Alcohol denat | up to 100 |
| EDTA | 0.001 |
| Ethylhexyl salicilate | 1.5 |
| Tris(Tetramethylhydroxypiperidinol) | 0.05 |
| Citrate (and) Aqua (and) Alcohol | |
| Water | 5 |
| Perfume | See Table 21 |
| Phase B | |
| Polyurethane-64 | See Table 21 |

TABLE 21
Different dosage of perfume and polyurethane-64
tested. Results are shown in FIG. 1 and FIG. 2

| | % Polyurethane-64 | % Perfume |
|---|---|---|
| Standard | 0 | 25 |
| (STD) | | |
| A | 0.4 | 23 |
| B | 2 | 20 |
| C | 2 | 23 |
| D | 0.8 | 20 |

### Other Test Methods:

### Sensory Glass Slides:

Samples of fragrance compositions and the controls were applied to glass slides (25 mm width) and were placed on a hot plate at 32° C. to represent skin temperature for varying durations. Glass slides of samples that were to be later compared were prepared at the same time.

Twenty microliters of a fragrance EDT product was carefully to glass slide allowing it to spread evenly.
Assessment when using primer solutions: 20 microliters of the primer was carefully added to the glass slide allowed to spread evenly. After waiting 4 minutes for solvent to evaporate, 20 microliters of the fragrance EDT were added on top of a primer.

Slides were coded so that their identity was not known by the experts. Samples were presented in the same tray, and panelists were able to compare both at the same moment. Panelists were selected from experienced evaluators among the industry and technical perfumers, which had been specifically trained to differentiate fragrance samples.

### Headspace Analysis

Ten microliters of samples of the compositions were applied at the tip of paper blotters (300 gram per m) and left evaporate at room temperature during two hours.

A tip was cut and sealed in a 20 ml tube, and headspace of the tube was left to stabilize for 10 minutes.

Then an SPME fiber was injected into the vial allowing for volatile molecules to be retained. Finally, the fiber was injected into inlet of a GC, gas chromatograph, and exposed to heating to desorb the molecules and move through the GC column.

### Equipment, Reagents, and Solutions:

Gas Chromatography equipped with capillary inlet system for capillary columns and mass detector
Apolar capillary column. DB-1 30 m×0.250 mm, film 0.25 micronm.
SPME fiber 65 micronmeter PDMS/DVB, fused silica 24 Ga N° 57310-U

### Chromatographic Conditions

| | DB-1 | |
|---|---|---|
| Injector | 240° | C. |
| Temperature | | |
| Injector mode | splitless | |
| Carrier gas | Helium | |
| Initial flow | 1.2 | ml/min |
| Initial | 90° | C. |
| Temperature | | |
| Temperature rate | 3.5° | C./min |
| Final | 260° | C. |
| Temperature | | |
| Final hold Time | 12 | minutes |
| Run Time | 60.57 | minutes |

MS Detector

| Solvent delay | 1.40 | minutes |
|---|---|---|
| EMV mode | relative | |
| Relative Voltage | 0 | |
| Low mass | 35 | |
| High mass | 350 | |
| MS source | 230° | C. |
| MS quad | 150° | C. |

### Use of Polyurethane-64 in a Primer Formulation

A primers containing polyurethane-64 and a modulator was used to enhance the perception of top notes from perfume. The primer from Example 13 was first allowed to evaporate solvent, and then the fragrance composition from Example 12 was applied.

Polyurethane-64 has been shown increase fragrance retention of top notes such as Dihydromyrcenol, Linalol and Linalyl acetate when used in combination with a fragrance modulator in a primer formulation (Table 24). Particularly, the use of fragrance accord in combination with Polyurethane-64 and a Modulator resulted in surprisingly good retention effect. Using an unscented primer (Example 14) to enhance fragrance retention was also effective, albeit at lower levels than when the primer contains a fragrance accord.

Sensory testing in glass slides up to t=8 hours after application consistently showed differences between Example 12, and Example 12 combined with the use Example 15 as a primer. The later was found richer in character, more floral, orange flower, jasmine, slightly more fruity, more powerful, and more citrus. The first was found only with remaining base notes. The application with a primer was always ranked as preferred versus the reference when targeting higher retention of topnotes and stronger citrus character.

### Example 12: Perfume

| Ingredient | Wt % |
|---|---|
| Alcohol denat | up to 100 |
| Parfum | 16 |
| Aqua | 6 |

### Example 13: Scented Primer with Citrus Accord, Polyurethane-64 and Modulator

| Ingredient | Wt % |
|---|---|
| Alcohol denat | up to 100 |
| Citrus Accord | 16 |
| Polyurethane-64 | 0.6 to 2.8 |
| Glucam P-20 | 5 to 15 |

### Example 14: Unscented Primer with Polyurethane-64 and Modulator

| Ingredient | Wt % |
|---|---|
| Alcohol denat | up to 100 |
| Polyurethane-64 | 0.6 to 2.8 |
| Glucam P-20 | 5 to 15 |

### Example 15: Perfume with Citrus Accord

| Ingredient | Wt % |
|---|---|
| Alcohol denat | up to 100 |
| Parfum | 16 |
| Citrus Accord | 16 |

TABLE 22
Fragrance constructions of fragrance oils

| Notes | Example 12 | Example 13 | Example 15 ¹ |
|---|---|---|---|
| Top | 16.17% | 77.3% | 46.7% |
| Middle | 23.62% | 21.6% | 22.6% |
| Base | 55.31% | 1.1% | 28.2% |
| Unknown | 4.9% | — | 2.5% |

¹ Example 15 is calculated from mixing the values of perfume from example 12 and example 13 in a 1:1 ratio

TABLE 23
Percentage of Dihydromyrenol, Linalol,
and Linalyl Acetate in fragrance oils

| PRM | Example 12 | Example 13 | Example 15 ¹ |
|---|---|---|---|
| Dihydromyrcenol | 1.9% | 10% | 5.9% |
| Linalol | 3.3% | 6.4% | 4.8% |
| Linalyl acetate | 4.8% | 13.2% | 9% |

¹ Example 15 is calculated from mixing the values of perfume oils from example 12 and example 13 in a 1:1 ratio

TABLE 24
Headspace Results at t = 2 hours expressed as number
of times Example 12 values at t = 2 hours

| | | Example 13 + | Example 14 + |
|---|---|---|---|
| | | Example 12 | Example 12 |
| | | (scented primer | (unscented primer |
| PRM | Example 15 | application) | application) |
| Dihydromyrcenol | 7.3 x | 13.3 x | 1.3 x |
| Linalol | 4.5 x | 6.7 x | 1.3 x |
| Linalyl acetate | 54 x | 68.9 x | 9.4 x |

TABLE 25
Ranking of perceived citrus note after 5 hours for different ratios of modulator
and polyurethane-64, in Example 15. Water was replaced by a combination of modulator
and polyurethane-64. The lower the number, the more citrus it was perceived.

| Ratio Glucam | Evaluator | Evaluator | Evaluator | Evaluator | Evaluator | |
|---|---|---|---|---|---|---|
| P-20:Polyrurethane-64 | 1 | 2 | 3 | 4 | 5 | Me [symbol] |
| 1.8:1 | 2 | 3 | 5 | 5 | 4 | [symbol] |
| 5.3:1 | 3 | 4 | 2 | 3 | 1 | [symbol] |
| 5.88:1 | 4 | 5 | 3 | 6 | 2 | [symbol] |
| 8.3:1 | 5 | 6 | 6 | 4 | 5 | |
| 12.5:1 | 1 | 2 | 1 | 1 | 3 | |
| 25:1 | 6 | 1 | 4 | 2 | 6 | |

a- Offnote perceived [symbol]
indicates data missing or illegible when filed

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A fragrance formulation comprising ethanol, water, one or more fragrances and a high molecular weight ingredient with at least one carbamate linkage, resulting from a reaction of an isocyanate and a polyol, the reaction comprising:

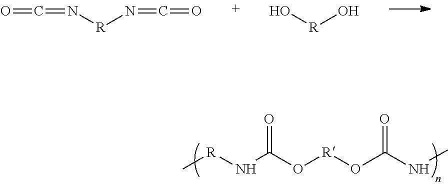

2. (canceled)

3. The fragrance formulation of claim 1, further comprising one or more colors.

4. (canceled)

5. (canceled)

6. (canceled)

7. (canceled)

8. (canceled)

9. (canceled)

10. A fragrance formulation comprising:
water;
ethanol and polyurethane-64;
a modulator component in a range of from about 0.1 wt % to about 30 wt % of the fragrance composition, the modulator being substantially non-odorous; and
a fragrance component present in an amount in a range of from about 0.04 wt % to about 40 wt % of the fragrance composition, the fragrance component 12. t comprising at least one of:
at least one low volatile fragrance material having a vapor pressure less than 0.001 Torr (0.000133 kPa) at 25° C.;
at least one moderate volatile fragrance material having a vapor pressure in the range of 0.1 Torr to 0.001 Torr (0.0133 kPa to 0.000133 kPa) at 25° C.; and at least one high volatile fragrance material having a vapor pressure greater than 0.1 Torr (0.0133 kPa) at 25° C., wherein the polyurethane 64 is a high molecular weight ingredient with at least one carbamate linkage, resulting from the reaction of an isocyanate and a polyol.

11. The fragrance composition of claim 10, wherein the modulator component is present in a range of from about 0.1 wt % to about 27 wt %.

12. The fragrance composition of claim 10, wherein the polyurethane-64 component is present in a range of about 0.24 to 0.6 percent by weight to produce less strong malodor than a corresponding fragrance composition having a greater concentration of polyurethan-64 component.

13. The fragrance formulation of claim 10, wherein the modulator is Hydrolite-5 Green, sustainable pentylene glycol.

14. The fragrance formulation of claim 10, wherein the modulator is Glucam P-20 Humectant, propoxylated methyl glucose ether.

15. The fragrance formulation of claim 10, wherein the modulator is Citropol F, polycitronellol acetate.

16. The fragrance composition of claim 10 wherein the modulator component comprises:
the compound according to Formula I: 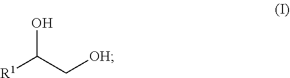
a repeating unit derived from the compound according to Formula II: 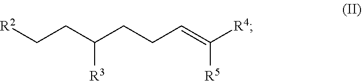
or
a mixture thereof, wherein
R¹, R², R³, R⁴, and R⁵, are independently chosen from —H, —OH, or substituted or unsubstituted (C₁-C₂₀)hydrocarbyl.

17. The fragrance composition of claim 15, wherein R¹, R², R³, R⁴, and R⁵, are independently chosen from (C₁-C₂₀)alkyl, (C₂-C₂₀)alkenyl, (C₃-C₂₀)cycloalkyl, (C₁-C₂₀)alkoxyl, (C₁-C₂₀)aryl, or a combination thereof.

18. The fragrance composition of claim 10, wherein the modulator component comprises pentylene glycol, polycitronellol, or a mixture thereof.

19. (canceled)

20. The fragrance composition of claim 10, wherein the modulator component comprises a mixture of pentylene glycol and polycitronellol and a molar ratio of pentylene glycol to polycitronellol is in a range of from about 2:1 to about 1:2.

21. (canceled)

22. The fragrance composition of claim 10, wherein a pH of the fragrance composition is in a range of from about 4 to about 8.

23. The fragrance composition of claim 10, wherein a pH of the fragrance composition is in a range of from about 5 to about 7.

24. The fragrance composition of claim 10, wherein the fragrance composition comprises less than about 78 wt % ethanol.

25. The fragrance composition of claim 10, wherein the fragrance composition comprises less than about 50 wt % ethanol.

26. (canceled)

27. (canceled)

28. (canceled)

29. The fragrance formulation of claim 10, wherein the smart gel includes polymer chains of terpolymer type which are constituted by poly(ethylene oxide) (PEO) and poly(propylene oxide) (PPO) of PEO-PPO-PEO form, which are modified at their termini by groups which can essentially be other chains of PEO-PPO-PEO, acid segments, amine groups or PEOs, these chains being linked to the terpolymer chains via chemical bridges which are constituted by urethane bridges, urea bridges, allophanate bridges and biuret bridges, and which include more than 80% by weight of said PEO-PPO-PEO.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The fragrance composition of claim 10 wherein the modulator comprises glucam.

46. (canceled)

47. The fragrance composition of claim 3, wherein the modulator component comprises a mixture of pentylene glycol and polycitronellol and a molar ratio of pentylene glycol to polycitronellol is in a range of from about 5:1 to about 1:5.

48. The fragrance composition of claim 3, wherein the modulator component comprises a mixture of pentylene glycol and polycitronellol and a molar ratio of pentylene glycol to polycitronellol is in a range of from about 2:1 to about 1:2.

49. A primer formulation comprising:
ethanol;
water;
polyurethane-64; and
one or more modulators.

50. The primer formulation of claim 49, wherein the modulator comprises glucam.

51. The fragrance composition of claim 49, wherein the weight ratio of modulator component to polyurethane-64 is in a range of from about 6:1 to about 25:1, most preferably about 12.5:1.
